SEQUENCE LISTING (37 sequences)

SEQ ID NO: 1 - protein, 30 aa, homo sapiens
RHRLHLAGFSSVRYAQGVNEDEIMLPSFHD

SEQ ID NO: 2 - protein, 25 aa, homo sapiens
SESSGNDPFTIVHENTGKCIQPLFD

SEQ ID NO: 3 - protein, 1723 aa, mus musculus
MRTGRVTPGLAAGLLLLLLRSFGLVEPSESSGNDPFTIVHENTGKCIQPLSDWVVAQDCSGTNNMLWKWVSQHRLFHLESQKCLGLDITKATDNLRMFSCDSTVMLWWKCEHHSLYTAAQYRLALKDGYAVANTNTSDVWKKGGSEENLCAQPYHEIYTRDGNSYGRPCEFPFLIGETWYHDCIHDEDHSGPWCATTLSYEYDQKWGICLLPESGCEGNWEKNEQIGSCYQFNNQEILSWKEAYVSCQNQGADLLSIHSAAELAYITGKEDIARLVWLGLNQLYSARGWEWSDFRPLKFLNWDPGTPVAPVIGGSSCARMDTESGLWQSVSCESQQPYVCKKPLNNTLELPDVWTYTDTHCHVGWLPNNGFCYLLANESSSWDAAHLKCKAFGADLISMHSLADVEVVVTKLHNGDVKKEIWTGLKNTNSPALFQWSDGTEVTLTYWNENEPSVPFNKTPNCVSYLGKLGQWKVQSCEKKLRYVCKKKGEITKDAESDKLCPPDEGWKRHGETCYKIYEKEAPFGTNCNLTITSRFEQEFLNYMMKNYDKSLRKYFWTGLRDPDSRGEYSWAVAQGVKQAVTFSNWNFLEPASPGGCVAMSTGKTLGKWEVKNCRSFRALSICKKVSEPQEPEEAAPKPDDPCPEGWHTFPSSLSCYKVFHIERIVRKRNWEEAERFCQALGAHLPSFSRREEIKDFVHLLKDQFSGQRWLWIGLNKRSPDLQGSWQWSDRTPVSAVMMEPEFQQDFDIRDCAAIKVLDVPWRRVWHLYEDKDYAYWKPFACDAKLEWVCQIPKGSTPQMPDWYNPERTGIHGPPVIIEGSEYWFVADPHLNYEEAVLYCASNHSFLATITSFTGLKAIKNKLANISGEEQKWWVKTSENPIDRYFLGSRRRLWHHFPMTFGDECLHMSAKTWLVDLSKRADCNAKLPFICERYNVSSLEKYSPDPAAKVQCTEKWIPFQNKCFLKVNSGPVTFSQASGICHSYGGTLPSVLSRGEQDFIISLLPEMEASLWIGLRWTAYERINRWTDNRELTYSNFHPLLVGRRLSIPTNFFDDESHFHCALILNLKKSPLTGTWNFTSCSERHSLSLCQKYSETEDGQPWENTSKTVKYLNNLYKIISKPLTWHGALKECMKEKMRLVSITDPYQQAFLAVQATLRNSSFWIGLSSQDDELNFGWSDGKRLQFSNWAGSNEQLDDCVILDTDGFWKTADCDDNQPGAICYYPGNETEEEVRALDTAKCPSPVQSTPWIPFQNSCYNFMITNNRHKTVTPEEVQSTCEKLHPKAHSLSIRNEEENTFVVEQLLYFNYIASWVMLGITYENNSLMWFDKTALSYTHWRTGRPTVKNGKFLAGLSTDGFWDIQSFNVIEETLHFYQHSISACKIEMVDYEDKHNGTLPQFIPYKDGVYSVIQKKVTWYEALNACSQSGGELASVHNPNGKLFLEDIVNRDGFPLWVGLSSHDGSESSFEWSDGRAFDYVPWQSLQSPGDCVVLYPKGIWRREKCLSVKDGAICYKPTKDKKLIFHVKSSKCPVAKRDGPQWVQYGGHCYASDQVLHSFSEAKQVCQELDHSATVVTIADENENKFVSRLMRENYNITMRVWLGLSQHSLDQSWSWLDGLDVTFVKWENKTKDGDGKCSILIASNETWRKVHCSRGYARAVCKIPLSPDYTGIAILFAVLCLLGLISLAIWFLLQRSHIRWTGFSSVRYEHGTNEDEVMLPSFHD

SEQ ID NO: 4 - protein, 30 aa, mus musculus
RSHIRWTGFSSVRYEHGTNEDEVMLPSFHD

SEQ ID NO: 5 - DNA, 5477 bp, homo sapiens
gaattccgggggcgggagccgcgtgcgcccgaggacccggccggaaggcttgcgccagctcaggatgaggacaggctgggcgacccctcgccgcccggcggggctcctcatgctgctcttctggttcttcgatctcgcggagccctctggccgcgcagctaatgaccccttcaccatcgtccatggaaatacgggcaagtgcatcaagccagtgtatggctggatagtagcagacgactgtgatgaaactgaggacaagttatggaagtgggtgtcccagcatcggctctttcatttgcactcccaaaagtgccttggcctcgatattaccaaatcggtaaatgagctgagaatgttcagctgtgactccagtgccatgctgtggtggaaatgtgagcaccactctctgtacggagctgcccggtaccggctggctctgaaggatggacatggcacagcaatctcaaatgcatctgatgtctggaagaaaggaggctcagaggaaagcctttgtgaccagccttatcatgagatctataccagagatgggaactcttatgggagaccttgtgaatttccattcttaattgatgggacctggcatcatgattgcattcttgatgaagatcatagtgggccatggtgtgccaccaccttaaattatgaatatgaccgaaagtggggcatctgcttaaagcctgaaaacggttgtgaagataattgggaaaagaacgagcagtttggaagttgctaccaatttaatactcagacggctctttcttggaaagaagcttatgtttcatgtcagaatcaaggagctgatttactgagcatcaacagtgctgctgaattaacttaccttaaagataaagaaggcattgctaagattttctggattggtttaaatcagctatactctgctagaggctgggaatggtcagaccacaaaccattaaactttctcaactgggatccagacaggcccagtgcacctactataggtggctccagctgtgcaagaatggatgctgagtctggtctgtggcagagcttttcctgtgaagctcaactgccctatgtctgcaggaaaccattaaataatacagtggagttaacagatgtctggacatactcagatacccgctgtgatgcaggctggctgccaaataatggattttgctatctgctggtaaatgaaagtaattcctgggataaggcacatgcgaaatgcaaagccttcagtagtgacctaatcagcattcattctctagcagatgtggaggtggttgtcacaaaactccataatgaggatatcaaagaagaagtgtggataggccttaagaacataaacataccaactttatttcagtggtcagatggtactgaagttactctaacatattgggatgagaatgagccaaatgttccctacaataagacgcccaactgtgtttcctacttaggagagctaggtcagtggaaagtccaatcatgtgaggagaaactaaaatatgtatgcaagagaaagggagaaaaactgaatgacgcaagttctgataagatgtgtcctccagatgagggctggaagagacatggagaaacctgttacaagatttatgaggatgaggtcccttttggaacaaactgcaatctgactatcactagcagatttgagcaagaatacctaaatgatttgatgaaaaagtatgataaatctctaagaaaatacttctggactggcctgagagatgtagattcttgtggagagtataactgggcaactgttggtggaagaaggcgggctgtaaccttttccaactggaattttcttgagccagcttccccgggcggctgcgtggctatgtctactggaaagtctgttggaaagtgggaggtgaaggactgcagaagcttcaaagcactttcaatttgcaagaaaatgagtggaccccttgggcctgaagaagcatcccctaagcctgatgacccctgtcctgaaggctggcagagtttccccgcaagtctttcttgttataaggtattccatgcagaaagaattgtaagaaagaggaactgggaagaagctgaacgattctgccaagcccttggagcacacctttctagcttcagccatgtggatgaaataaaggaatttcttcactttttaacggaccagttcagtggccagcattggctgtggattggtttgaataaaaggagcccagatttacaaggatcctggcaatggagtgatcgtacaccagtgtctactattatcatgccaaatgagtttcagcaggattatgacatcagagactgtgctgctgtcaaggtatttcataggccatggcgaagaggctggcatttctatgatgatagagaatttatttatttgaggccttttgcttgtgatacaaaacttgaatgggtgtgccaaattccaaaaggccgtactccaaaaacaccagactggtacaatccagagcgtgctggaattcatggacctccacttataattgaaggaagtgaatattggtttgttgctgatcttcacctaaactatgaagaagccgtcctgtactgtgccagcaatcacagctttcttgcaactataacatcttttgtgggactaaaagccatcaaaaacaaaatagcaaatatatctggtgatggacagaagtggtggataagaattagcgagtggccaatagatgatcattttacatactcacgatatccatggcaccgctttcctgtgacatttggagaggaatgcttgtacatgtctgccaagacttggcttatcgacttaggtaaaccaacagactgtagtaccaagttgcccttcatctgtgaaaaatataatgtttcttcgttagagaaatacagcccagattctgcagctaaagtgcaatgttctgagcaatggattccttttcagaataagtgttttctaaagatcaaacccgtgtctctcacattttctcaagcaagcgatacctgtcactcctatggtggcacccttccttcagtgttgagccagattgaacaagactttattacatccttgcttccggatatggaagctactttatggattggtttgcgctggactgcctatgaaaagataaacaaatggacagataacagagagctgacgtacagtaactttcacccattattggttagtgggaggctgagaataccagaaaatttttttgaggaagagtctcgctaccactgtgccctaatactcaacctccaaaaatcaccgtttactgggacgtggaattttacatcctgcagtgaacgccactttgtgtctctctgtcagaaatattcagaagttaaaagcagacagacgttgcagaatgcttcagaaactgtaaagtatctaaataatctgtacaaaataatcccaaagactctgacttggcacagtgctaaaagggagtgtctgaaaagtaacatgcagctggtgagcatcacggacccttaccagcaggcattcctcagtgtgcaggcgctccttcacaactcttccttatggatcggactcttcagtcaagatgatgaactcaactttggttggtcagatgggaaacgtcttcattttagtcgctgggctgaaactaatgggcaactcgaagactgtgtagtattagacactgatggattctggaaaacagttgattgcaatgacaatcaaccaggtgctatttgctactatccaggaaatgagactgaaaaagaggtcaaaccagttgacagtgttaaatgtccatctcctgttctaaatactccgtggataccatttcagaactgttgctacaatttcataataacaaagaataggcatatggcaacaacacaggatgaagttcatactaaatgccagaaactgaatccaaaatcacatattctgagtattcgagatgaaaaggagaataactttgttcttgagcaactgctgtacttcaattatatggcttcatgggtcatgttaggaataacttatagaaataattctcttatgtggtttgataagaccccactgtcatatacacattggagagcaggaagaccaactataaaaaatgagaggtttttggctggtttaagtactgacggcttctgggatattcaaacctttaaagttattgaagaagcagtttattttcaccagcacagcattcttgcttgtaaaattgaaatggttgactacaaagaagaatataatactacactgccacagtttatgccatatgaagatggtatttacagtgttattcaaaaaaaggtaacatggtatgaagcattaaacatgtgttctcaaagtggaggtcacttggcaagcgttcacaaccaaaatggccagctctttctggaagatattgtaaaacgtgatggatttccactatgggttgggctctcaagtcatgatggaagtgaatcaagttttgaatggtctgatggtagtacatttgactatatcccatggaaaggccaaacatctcctggaaattgtgttctcttggatccaaaaggaacttggaaacatgaaaaatgcaactctgttaaggatggtgctatttgttataaacctacaaaagctaaaaagctgtcccgtcttacatattcatcaagatgtccagcagcaaaagagaatgggtcacggtggatccagtacaagggtcactgttacaagtctgatcaggcattgcacagtttttcagaggccaaaaaattgtgttcaaaacatgatcactctgcaactatcgtttccataaaagatgaagatgagaataaatttgtgagcagactgatgagggaaaataataacattaccatgagagtttggcttggattatctcaacattctgttgaccagtcttggagttggttagatggatcagaagtgacatttgtcaaatgggaaaataaaagtaagagtggtgttggaagatgtagcatgttgatagcttcaaatgaaacttggaaaaaagttgaatgtgaacatggttttggaagagttgtctgcaaagtgcctctgggccctgattacacagcaatagctatcatagttgccacactaagtatcttagttctcatgggcggactgatttggttcctcttccaaaggcaccgtttgcacctggcgggtttctcatcagttcgatatgcacaaggagtgaatgaagatgagattatgcttccttctttccatgactaaattcttctaaaagttttctaatttgcactaatgtgttatgagaaattagtcacttaaaatgtccagtgtcagtatttactctgctccaaagtagaactcttaaatactttttcagttgtttagatctaggcatgtgctggtatccacagttaattccctgctaaatgccatgtttatcaccctaattaatagaatggaggggactccaaagctggaactgaagtcaaattgtttgacagtaata

SEQ ID NO: 6 - protein, 1825 aa, homo sapiens
  VARIANT 1744, 1751, 1763, 1785, 1787, 1795, 1807, 1808: Xaa = Any Amino Acid
NSGGGSRVRPRTRPEGLRQLRMRTGWATPRRPAGLLMLLFWFFDLAEPSGRAANDPFTIVHGNTGKCIKPVYGWIVADDCDETEDKLWKWVSQHRLFHLHSQKCLGLDITKSVNELRMFSCDSSAMLWWKCEHHSLYGAARYRLALKDGHGTAISNASDVWKKGGSEESLCDQPYHEIYTRDGNSYGRPCEFPFLIDGTWHHDCILDEDHSGPWCATTLNYEYDRKWGICLKPENGCEDNWEKNEQFGSCYQFNTQTALSWKEAYVSCQNQGADLLSINSAAELTYLKDKEGIAKIFWIGLNQLYSARGWEWSDHKPLNFLNWDPDRPSAPTIGGSSCARMDAESGLWQSFSCEAQLPYVCRKPLNNTVELTDVWTYSDTRCDAGWLPNNGFCYLLVNESNSWDKAHAKCKAFSSDLISIHSLADVEVVVTKLHNEDIKEEVWIGLKNINIPTLFQWSDGTEVTLTYWDENEPNVPYNKTPNCVSYLGELGQWKVQSCEEKLKYVCKRKGEKLNDASSDKMCPPDEGWKRHGETCYKIYEDEVPFGTNCNLTITSRFEQEYLNDLMKKYDKSLRKYFWTGLRDVDSCGEYNWATVGGRRRAVTFSNWNFLEPASPGGCVAMSTGKSVGKWEVKDCRSFKALSICKKMSGPLGPEEASPKPDDPCPEGWQSFPASLSCYKVFHAERIVRKRNWEEAERFCQALGAHLSSFSHVDEIKEFLHFLTDQFSGQHWLWIGLNKRSPDLQGSWQWSDRTPVSTIIMPNEFQQDYDIRDCAAVKVFHRPWRRGWHFYDDREFIYLRPFACDTKLEWVCQIPKGRTPKTPDWYNPERAGIHGPPLIIEGSEYWFVADLHLNYEEAVLYCASNHSFLATITSFVGLKAIKNKIANISGDGQKWWIRISEWPIDDHFTYSRYPWHRFPVTFGEECLYMSAKTWLIDLGKPTDCSTKLPFICEKYNVSSLEKYSPDSAAKVQCSEQWIPFQNKCFLKIKPVSLTFSQASDTCHSYGGTLPSVLSQIEQDFITSLLPDMEATLWIGLRWTAYEKINKWTDNRELTYSNFHPLLVSGRLRIPENFFEEESRYHCALILNLQKSPFTGTWNFTSCSERHFVSLCQKYSEVKSRQTLQNASETVKYLNNLYKIIPKTLTWHSAKRECLKSNMQLVSITDPYQQAFLSVQALLHNSSLWIGLFSQDDELNFGWSDGKRLHFSRWAETNGQLEDCVVLDTDGFWKTVDCNDNQPGAICYYPGNETEKEVKPVDSVKCPSPVLNTPWIPFQNCCYNFIITKNRHMATTQDEVHTKCQKLNPKSHILSIRDEKENNFVLEQLLYFNYMASWVMLGITYRNNSLMWFDKTPLSYTHWRAGRPTIKNERFLAGLSTDGFWDIQTFKVIEEAVYFHQHSILACKIEMVDYKEEYNTTLPQFMPYEDGIYSVIQKKVTWYEALNMCSQSGGHLASVHNQNGQLFLEDIVKRDGFPLWVGLSSHDGSESSFEWSDGSTFDYIPWKGQTSPGNCVLLDPKGTWKHEKCNSVKDGAICYKPTKAKKLSRLTYSSRCPAAKENGSRWIQYKGHCYKSDQALHSFSEAKKLCSKHDHSATIVSIKDEDENKFVSRLMRENNNITMRVWLGLSQHSVDQSWSWLDGSEVTFVKWENKSKSGVGRCSMLIASNETWKKVECEHGFGRVVCKVPLGPDYTAIAIIVATLSILVLMGGLIWFLFQRHRLHLAGFSSVRYAQGVNEDEIMLPSFHDXILLKVFXFALMCYEKLVTXNVQCQYLLCSKVELLNTFSVVXIXACAGIHSXFPAKCHVYHPNXXNGGDSKAGTEVKLFDSN

SEQ ID NO: 7 - DNA, 49 bp, Artificial Sequence
  synthetic
atagtttagcggccgcgatatctcactaacactcattcctgttgaagct

SEQ ID NO: 8 - DNA, 57 bp, Artificial Sequence
  synthetic
atagtttagcggccgctcactagctagctttaccaggagagtgggagagactcttct

SEQ ID NO: 9 - DNA, 68 bp, Artificial Sequence
  synthetic
ctagcgacatggccaagaaggagacagtctggaggctcgaggagttcggtaggttcacaaacaggaac

SEQ ID NO: 10 - DNA, 71 bp, Artificial Sequence
  synthetic
acagacggtagcacagactatggtattctccagattaacagcaggtattatgacggtaggacatgataggc

SEQ ID NO: 11 - DNA, 70 bp, Artificial Sequence
  synthetic
gctgtaccggttcttcctctgtcagacctccgagctcctcaagccatccaagtgtttgtccttgtgtctg

SEQ ID NO: 12 - DNA, 69 bp, Artificial Sequence
  synthetic
ccatcgtgtctgataccataagaggtctaattgtcgtccataatactgccatcctgtactatccgccgg

SEQ ID NO: 13 - DNA, 330 bp, homo sapiens
caggctgttgtgactcaggaatcagcactcaccacatcacctggtgaaacagtcacactcacttgtcgctcaagtactggggctgttacaattagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatctatttctgtgctctatggtacaacaaccagtttattttcggcagtggaaccaaggtcactgtcctaggt

SEQ ID NO: 14 - DNA, 354 bp, homo sapiens
gaggtccagctgcaacagtctggacctgtgctggtgaagcctggggcttcagtgaagatgtcctgtaaggcttctggaaacacattcactgactcctttatgcactggatgaaacagagccatggaaagagtcttgagtggattggaattattaatccttataacggcggtactagctacaaccagaaattcaagggcaaggccacattgactgttgacaagtcctccagcacagcctacatggagctcaacagcctgacatctgaggactctgcagtctattactgtgcaagaaacggggtgcggtactactttgactactggggccaaggcaccactctcacagtctcctca

SEQ ID NO: 15 - protein, 8 aa, mus musculus
SIINFEKL

SEQ ID NO: 16 - protein, 17 aa, mus musculus
LSQAVHAAHAEINEAGR

SEQ ID NO: 17 - DNA, 8858 bp, homo sapiens
gacggatcgggagatctgctagcccgggtgacctgaggcgcgccggcttcgaatagccagagtaacctttttttttaattttattttattttatttttgagatggagtttggcgccgatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccatggaagccccagctcagcttctcttcctcctgctactctggctcccagataccaccggagacattgttctgactcagtctccagccaccctgtctgtgactccaggagatagagtctctctttcctgcagggccagccagagtattagcgactacttacactggtatcaacaaaaatcacatgagtctccaaggcttctcatcaaatatgcttcccattccatctctgggatcccctccaggttcagtggcagtggatcagggtcagatttcactctcagtatcaacagtgtggaacctgaagatgttggaatttattactgtcaacatggtcacagctttccgtggacgttcggtggaggcaccaagctggaaatcaaacgtaagtctcgagtctctagataaccggtcaatcggtcaatcgattggaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgcccttatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcacgtaaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccaacgcagctggtaagtgggggcctgggatcaaatagctacctgcctaatcctgcccwcttgagccctgaatgagtctgccttccagggctcaaggtgctcaacaaaacaacaggcctgctattttcctggcatctgtgccctgtttggctagctaggagcacacatacatagaaattaaatgaaacagaccttcagcaaggggacagaggacagaattaaccttgcccagacactggaaacccatgtatgaacactcacatgtttgggaagggggaagggcacatgtaaatgaggactcttcctcattctatggggcactctggccctgcccctctcagctactcatccatccaacacacctttctaagtacctctctctgcctacactctgaaggggttcaggagtaactaacacagcatcccttccctcaaatgactgacaatccctttgtcctgctttgtttttctttccagtcagtactgggaaagtggggaaggacagtcatggagaaactacataaggaagcaccttgcccttctgcctcttgagaatgttgatgagtatcaaatctttcaaactttggaggtttgagtaggggtgagactcagtaatgtcccttccaatgacatgaacttgctcactcatccctgggggccaaattgaacaatcaaaggcaggcataatccagttatgaattcttgcggccgcttgctagcttcacgtgttggatccaaccgcggaagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccgggcctctcaaaaaagggaaaaaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccaccttagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 18 - DNA, 744 bp, homo sapiens
gaggtgcagctgttggagtctgggggaggcttggtacagcctggggggtccctgagactctcctgtgcagcctctggattcaccttcagtgactactacatgagctgggtccgccaggctccagggaaggggctggagtgggtctcagcaattagtggtagtggtgggagcacatactacgcagactccctgaagggccggttcaccatctccagagacaattccaagaacacgctgtatctgcaaatgaacagcctgagagccgaggacacggccgtgtattactgtgcgagagcccctgttgactacagtaacccctccggtatggacgtctggggccaaggtacactggtcaccgtgagcagcggtggaggcggttcaggcggaggtggatccggcggtggcggatcgcagtctgtgctgactcagccaccctcagcgtctgggacccccgggcagagggtcaccatctcttgttctggaagcagctccaacatcggaagtaatactgttaactggtatcagcagctcccaggaacggcccccaaactcctcatctatagggataatcagcgaccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtcctaggt

SEQ ID NO: 19 - DNA, 630 bp, homo sapiens
ggtctatataagcagagctgggtacgtcctcacattcagtgatcagcactgaacacagacccgtcgacggtgatcaggactgaacagagagaactcaccatggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatggggggtactatggttcggggagttatgggtactttgactactggggccagggaaccctggtcaccgtctcctcagctagcaccaagggcccatcggtcttccccctggcaccctcctccaagagcacctctgggggcacagcggccctgggctgcctggtcaaggactacttcccc

SEQ ID NO: 20 - DNA, 8953 bp, human immunodeficiency virus
  misc_feature 7216: n = A,T,C or G
ttctctcgacgcaggactcggcttgctgaagtgcacacggcaagaggcgaagggcggcgactggtgagtacgccaaaaatattttttgactagcggaggctagaaggagagagatgggtgcgagagcgtcaatattaagtgggggaaaattagacgattgggaaaaaattcggttgaggccagggggaaagaaacaatataggataaaacatctagtatgggcaagcagggagctggacagatttgcacttaaccctggccttctagagtcagcaaaaggctgtcaacaaatactagtacagctccaaccagctcttcagacaggaacagaagaaattaaatcattatataatacagtagcaaccctctattgcgtacatcagaggatagagataaaagacaccaaggaagctttagacaagatagaggaaattcaaaacaaaaacaaacagcagacacagaaagcagaaactgacaaaaaagacaacagtcaggtcagtcaaaattatcctatagtgcagaatctgcaagggcaaccggtacaccaggccttatcacctagaactttaaatgcatgggtaaaagtgatagaagagaaagccttcagcccagaagtgatacccatgttttcagcattatcagaaggagccaccccgcaagatttaaacaccatgctaaacacaatagggggacaccaagcagctatgcaaatgttaaaagatactatcaatgaggaagctgcagaatgggacagggtacatccagtacatgcagggcctgttgcaccaggccaggtgagagaaccaaggggaagtgatatagcaggaactactagtaacctccaggaacaaataggatggatgacaggcaacccaccgatcccagtaggagaaatttataaaaggtggataattctgggactaaataaaatagtgagaatgtatagccctgtcagcattttggatataagacaaggaccaaaagaacctttcagagactatgtagacagattctttaaagctctaagagctgagcaagctacacaggatgtaaaaaattggatgacagataccttgttggtccaaaatgcaaatccagattgcaagaccattttaaaagcattaggatcaggagctacactagaagaaatgatgacagcatgtcagggagtgggaggacctggtcataaagcaagagttttggctgaagcaatgagccaagtgaccaatacaaacataatgatgcaaagaggtaactttagggatcataaaagaattgttaagtgtttcaattgtggcaaacaaggacacatagcaaaaaactgcagggcccctagaaaaaagggctgttggaaatgtggaaaggaaggacaccaaatgaaagactgcactgagagacaggctaattttttagggaagatttggccttccagcaaagggaggccagggaattttctccagagcagaccagagccaacagccccaccagcagagagcctcgggttcggagaggagatcccctccccgaaacaggagccgaaggacaaggaactgtatcctctaacttccctcagatcactctttggcagcgaccccttgtcacaataagaataggggggcagctaagggaagctctattagatacaggagcagacgatacagtattagaagaaatagatttgccaggaaaatggaaaccaaaaatgatagggggaattggaggttttatcaaagtgagacagtataatgaggtacccatagaaattgagggaaaaaaggctataggtacagtattaataggacctacacctgtcaacataattggaagaaacatgttgactcagcttggttgtactttaaattttccaattagtcctattgaaactgtaccagtaaaattaaagccaggaatggatggcccaaaaattaaacaatggccattgacagaagaaaaaataaaagcattaacacaaatttgtgcagaactggaagaggagggaaaaatttcaagaattgggcctgaaaatccatataacaccccagtatttgccataaagaaaaaagacagtactaaatggagaaaattagtagattttagagaactcaataaaagaactcaagacttctgggaagttcagttaggaataccacatccagcagggttaaaaaagaaaaaatcagtcacagtactggatgtgggggatgcatatttttcagtccctttatatgaagatttcaggaagtatactgcattcactatacctagtataaacaatgagacaccagggatcagatatcagtacaacgtgctaccacagggatggaaaggatcaccagcaatatttcagtgtagcatgacaaaaatcttaaaaccttttagagaaagaaacccagaaatagttatctaccagtacatggatgacttgtatgtgggatctgacttggaaatagaacagcatagaagaaaaataaaggagctgagggaacatctattgaagtggggattttacacaccagataaaaaacatcagaaagaacctccatttctttggatgggatatgagctccatcctgacaaatggacagtacaacctatacagctgccagaaaaagaagattggactgtcaatgatatacaaaagttagtgggaaaactaaattgggcaagtcaaatttatccaggaattaaaataaaggaactatgtaaactcattaggggggctaaagcactaacagacatagtaccattgactagagaagcagaattggaactggcagaaaacaaggagattctaaaagaaccagtacatggggtatattatgacccagcaagagaattaatagcagaagtgcagaaacaaggactggaccaatggacatatcaaatttatcaggagccatttaaaaacctgaaaacagggaaatatgcaaaaaggaggagtgcccacactaatgatgtaaagcaattatcacaagtggtgcaaaaaatagccttggaagccatagtgatatggggaaaaactcctaaatttagactacccatacaaaaggaaacatgggagacatggtggacagactattggcaggccacctggattcctgagtgggagtttgtcaataccccccctctagtaaaattatggtaccaattagaaaaggaacccataatgggagcagaaactttctatgtagatggggcatctaacagggaaactaaagtaggaaaggcagggtatgttactgacaaaggaagacagaaagtaattaccctaactgacacaacaaatcagaagactgaactacaagccatttatttagctttacaggattcagggatagaagtaaacatagtaacagattcacaatatgcattggggattattcaagcacaaccagataagagtgaatcagaattagtcaatcaaataatagaggagttaataaagaaggaaaaggtctacctgtcgtgggtaccagcacacaaaggaattggaggaaatgaacaagtagataaattagtcagttctggaatcaggaaagtgctgtttctagatgggatagataaagctcaagaagaacatgaaaaatatcatagcaattggagagcaatggctagtgattttaatctaccacctgtagtagcaaaagaaatagtagctagctgtgataaatgtcagctaaagggggaagccatgcatggacaagtagactgtagtccagggatatggcaattagattgtacacatttagaaggaaaagttatcctggtagcagttcatgtagccagtggctatatagaagcagaagttatcccagcagaaacaggacaggaagcagcattttttatattaaaattagcaggcggatggccagtaaaagcaatacatacagataatggcagcaacttcaccagtggtgctgtgaaggcagcctgttggtgggcagatatcaaacaggaatttggaattccctacaatccccaaagtcaaggagtagtagaatctatgaataaagaattaaagaaaatcataggacaggtaagagaacaagctgaacaccttaagacagcagtacagatggcagtattcatacacaattttaaaagaaaaggggggattgggggatacagtgcaggggaaagaataatagacataatagcaacagacatacaaactaaagaattacaaaaacaaatcacaaaaattcaaaattttcgggtttattacagggacagcagagacccaatttggaaaggaccagcaaaactgctctggaaaggtgaaggggcagtagtaatacaagacaatagtgaaataaaggtagtaccaagaagaaaagcaaagatcattagagattatggaaaacagatggcaggtgatgattgtgtggcaggtagacaggatgaggattaacacatggaaaagtttagtaaagtaccatatgaatgtttcaaagaaagctagacaatggctgtatagacatcactatgatagccgtcatccaaaaataagttcagaagtacacatcccactaggagaggctagattagtagtaacaacatattggggtctgcaaacaggagaaagagattggcacttgggtcagggagtctccatagaatggaggcggaaaaggtacagaacacaagtagaccctggcctggcagaccaactaattcatatgcattactttgattgtttttcagactctgccataaggaaggccatattaggacaaatagttagccctaggtgtgactaccaagcaggacataacaaggtaggatctctacaatatctggcattaacagcattaataaaaccaaaaaggagaaagccacctttgcctagtgttcagaaactagtagaggatagatggaacaagccccagaagaccagggaccacagagagagccataccatgaatggacactagagcttttggaggagcttaaaaatgaagctgttagacactttcctaggccatggctccatggtttaggacagtatgtctatagcacttatggagatacatgggaaggagtcgaagccgtaataagaatactgcaacaactattgtttattcatttcagaatcgggtgccatcatagcagaataggcattataccacagagaagagggaggaatggagccagtagatcctaacagagagccctggaaccatccaggaagtcagcctaaaactgcttgtactaattgttattgtaaaaagtgttgctatcattgtcaagtgtgctttctacagaagggcttaggcatttcctatggcaggaagaagcggagacaacgacgatcagctcctcctggcagtaagaatcatcaagatcttataccagagcagtaagtaacttaattagcatatgtaatggtatctttacaaatagtagcaatagtagcattaatagtagcatttttccttgcaatatgtgtgtggactatagtgtatatagaatataagaaactgttaagacaaaggaaaatagataagttaattaatagaataagagaaagggcagaagacagtggtaacgagagtgatggagacacagacgagttggctgagcttgtggagatggggcctcatgatctttggaatgttaatgatttgtagtgctagagaaaacttgtgggtcacagtctattatggggtacctgtatggagagatgcaaagaccactttattttgcgcatctgatgctaaagcatatagtactgaaaaacataatgtctgggctacacatgcttgtgtacccacagatccaaacccacaagagatgagtctgccaaatgtaacagaaaattttaacatgtggaaaaatgacatggtagaccagatgcaggaagatataatcagtgtatgggatgaaagcttaaagccatgtgtaaagataacccctctctgtgtcactttaaattgtagcgacgtcaatagtaacaatagtacagatagtaatagtagtgcaagcaacaatagtcctgaaatcatgaaaaactgctctttcaatgtaactacagaaataagaaataaaaggaagcaagaatacgcgcttttctatagacaagatgtagtaccaattaatagtgacaataaaagttatattctaataaactgtaatacctcagttattaaacaggcttgtccaaaggtgtcttttcaaccaattcccatacattattgtgctccagctggttttgcgattctaaaatgtaataataagactttcaatggaacaggaccatgcaaaaatgtcagtacagtacaatgtacacacggaattaagccagtggtatcaactcaactactgctaaatggcagtgtagcagaaggagacataataattagatctgaaaatatctcagacaatgctaaaaacataatagtacaacttaatgacactgtagaaattgtgtgtaccagacctaataacaatacaagaaaaggtatacacatgggaccaggacaagtgctctacgcaacaggggaaataataggagatataaggaaagcatattgtaacattagtagaaaagattggaataacactttacgtagagtagctaaaaaactaagagaacactttaataaaacaatagactttacatcaccctcaggaggggacatagaaattacaacacatagttttaattgtggaggagaatttttctattgtaatacatcaacactgttcaatagtagttgggatgagaataacattaaggacacaaatagtacaaatgacaacacaactatcacaataccatgtaaaataaaacaaattgtgagaatgtggcaaagaacaggacaagcaatatatgcccctcccatcgcaggaaacattacatgcaaatcaaatattacaggattattattgacacgtgatggaggaaacaggaatggcagtgagaatggcactgagaccttcagacctacaggaggaaatatgaaagataattggagaagtgaattatataaatataaagtagtagagcttgagccactaggagtagcacccaccaaggcaaaaagaagagtggtggagagagaaaaaagagcagtgggaataggagctgtgttccttgggttcttgggaacagcaggaagcactatgggcgcagcgtcaataacgctgacggtacaggtcagacaattgttgtctggcatagtgcaacagcaaagcaatttgctgaaggctatanaagcgcaacagcatctgttgaagctcactgtctggggcattaagcagctccaggcaagagtcctggctgtggaaagatacctaaaggatcaacagctcctaggaatttggggctgctctggaaaactcatctgcaccactaatgtgccctggaatgctagttggagtaataaatcttatgaggacatttgggagaacatgacctggatacaatgggaaagggaaattaacaattacacaggaataatatacagtctaattgaagaagcacaaaaccagcaggaaactaatgaaaaggacttattggcattggacaagtggacaaatttgtggaattggtttaacatatcaaactggctgtggtacataaaaatattcataatgataataggaggcttaataggtttaagaataatttttgctgtgcttgctatagtaaatagagttaggcagggatactcacctttgtcatttcagacccttattccaaacccaacggaagccgacaggcccggaggaatcgaagaaggaggtggagagcaaggcagaaccagatcgattcgattagtgaacggattcttagctcttgcctgggacgacctgcggagcctgtgcctcttcagttaccaccgattgagagacttcgtcttgattgcagcgaggactgtgggaactctgggactcagggggtgggagatcctcaaatatctggtgaaccttgtatggtattgggggcaggaactaaagaatagtgctattagtttgcttaataccacagcaatagcagtagctgaaggaacagatagaatcatagaaatagcacaaagagcttttagagctattcttcacatacctagaagaataagacagggtttagaaagagctttgctataaaatggggaacaagtggtcaaaaagttggcctcaggtaagggacagaatgaggcgagctgctcctgctccagcagcagatggagtgggagcagtgtctcaagatttggctaagcatggggcaatcacaagcagcaatacagcagctacaaatgatgactgtgcctggctggaagcacaaacagaggaggaggttggatttccagtcagacctcaggtwccattaagaccaatgacatacaaaggagcttttgatcttagcttctttttaaaagaaaaggggggactggatgggttaatttactccaagaaaagacaagagatccttgatctgtgggttcataacacacaaggttacttccctgactggcaaaactacacaccagggccagggaccabatacccattgacatttggatggtgcttcaagctagtaccagttgatccaagcgaagtagaggaagctaatgaaggagagaacaactgcctgttacaccccgcatgccagcatggaatagaggatgaagaaagagaagtgctaaagtggaagtttgacagctccctagcacggagacacatagcccgagagctacatccggagttttacaaagactgctgacaaagaagtttctagcggggactttccgctggggactttccaggggaggtgtggcctgggcggggttggggagtggctaaccctcagatgctgcatataagcagctgcttttcgcttgtactgggtctctcttgttagaccagatctgagcctgggagctctctggctaactagggaacccactgcttaagcctcaataaagcttgccttgagggcgcatgcaagccg

SEQ ID NO: 21 - protein, 497 aa, human immunodeficiency virus gag protein
MGARASILSGGKLDDWEKIRLRPGGKKQYRIKHLVWASRELDRFALNPGLLESAKGCQQILVQLQPALQTGTEEIKSLYNTVATLYCVHQRIEIKDTKEALDKIEEIQNKNKQQTQKAETDKKDNSQVSQNYPIVQNLQGQPVHQALSPRTLNAWVKVIEEKAFSPEVIPMFSALSEGATPQDLNTMLNTIGGHQAAMQMLKDTINEEAAEWDRVHPVHAGPVAPGQVREPRGSDIAGTTSNLQEQIGWMTGNPPIPVGEIYKRWIILGLNKIVRMYSPVSILDIRQGPKEPFRDYVDRFFKALRAEQATQDVKNWMTDTLLVQNANPDCKTILKALGSGATLEEMMTACQGVGGPGHKARVLAEAMSQVTNTNIMMQRGNFRDHKRIVKCFNCGKQGHIAKNCRAPRKKGCWKCGKEGHQMKDCTERQANFLGKIWPSSKGRPGNFLQSRPEPTAPPAESLGFGEEIPSPKQEPKDKELYPLTSLRSLFGSDPLSQ

SEQ ID NO: 22 - protein, 1001 aa, human immunodeficiency virus pol protein
FFREDLAFQQREAREFSPEQTRANSPTSREPRVRRGDPLPETGAEGQGTVSSNFPQITLWQRPLVTIRIGGQLREALLDTGADDTVLEEIDLPGKWKPKMIGGIGGFIKVRQYNEVPIEIEGKKAIGTVLIGPTPVNIIGRNMLTQLGCTLNFPISPIETVPVKLKPGMDGPKIKQWPLTEEKIKALTQICAELEEEGKISRIGPENPYNTPVFAIKKKDSTKWRKLVDFRELNKRTQDFWEVQLGIPHPAGLKKKKSVTVLDVGDAYFSVPLYEDFRKYTAFTIPSINNETPGIRYQYNVLPQGWKGSPAIFQCSMTKILKPFRERNPEIVIYQYMDDLYVGSDLEIEQHRRKIKELREHLLKWGFYTPDKKHQKEPPFLWMGYELHPDKWTVQPIQLPEKEDWTVNDIQKLVGKLNWASQIYPGIKIKELCKLIRGAKALTDIVPLTREAELELAENKEILKEPVHGVYYDPARELIAEVQKQGLDQWTYQIYQEPFKNLKTGKYAKRRSAHTNDVKQLSQVVQKIALEAIVIWGKTPKFRLPIQKETWETWWTDYWQATWIPEWEFVNTPPLVKLWYQLEKEPIMGAETFYVDGASNRETKVGKAGYVTDKGRQKVITLTDTTNQKTELQAIYLALQDSGIEVNIVTDSQYALGIIQAQPDKSESELVNQIIEELIKKEKVYLSWVPAHKGIGGNEQVDKLVSSGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQEAAFFILKLAGGWPVKAIHTDNGSNFTSGAVKAACWWADIKQEFGIPYNPQSQGVVESMNKELKKIIGQVREQAEHLKTAVQMAVFIHNFKRKGGIGGYSAGERIIDIIATDIQTKELQKQITKIQNFRVYYRDSRDPIWKGPAKLLWKGEGAVVIQDNSEIKVVPRRKAKIIRDYGKQMAGDDCVAGRQDED

SEQ ID NO: 23 - protein, 101 aa, human immunodeficiency virus tat protein
MEPVDPNREPWNHPGSQPKTACTNCYCKKCCYHCQVCFLQKGLGISYGRKKRRQRRSAPPGSKNHQDLIPEQPLFQTQRKPTGPEESKKEVESKAEPDRFD

SEQ ID NO: 24 - protein, 116 aa, human immunodeficiency virus rev protein
MAGRSGDNDDQLLLAVRIIKILYQSNPYSKPNGSRQARRNRRRRWRARQNQIDSISERILSSCLGRPAEPVPLQLPPIERLRLDCSEDCGNSGTQGVGDPQISGEPCMVLGAGTKE

SEQ ID NO: 25 - protein, 850 aa, human immunodeficiency virus env protein
  VARIANT 554: Xaa = Any Amino Acid
METQTSWLSLWRWGLMIFGMLMICSARENLWVTVYYGVPVWRDAKTTLFCASDAKAYSTEKHNVWATHACVPTDPNPQEMSLPNVTENFNMWKNDMVDQMQEDIISVWDESLKPCVKITPLCVTLNCSDVNSNNSTDSNSSASNNSPEIMKNCSFNVTTEIRNKRKQEYALFYRQDVVPINSDNKSYILINCNTSVIKQACPKVSFQPIPIHYCAPAGFAILKCNNKTFNGTGPCKNVSTVQCTHGIKPVVSTQLLLNGSVAEGDIIIRSENISDNAKNIIVQLNDTVEIVCTRPNNNTRKGIHMGPGQVLYATGEIIGDIRKAYCNISRKDWNNTLRRVAKKLREHFNKTIDFTSPSGGDIEITTHSFNCGGEFFYCNTSTLFNSSWDENNIKDTNSTNDNTTITIPCKIKQIVRMWQRTGQAIYAPPIAGNITCKSNITGLLLTRDGGNRNGSENGTETFRPTGGNMKDNWRSELYKYKVVELEPLGVAPTKAKRRVVEREKRAVGIGAVFLGFLGTAGSTMGAASITLTVQVRQLLSGIVQQQSNLLKAIXAQQHLLKLTVWGIKQLQARVLAVERYLKDQQLLGIWGCSGKLICTTNVPWNASWSNKSYEDIWENMTWIQWEREINNYTGIIYSLIEEAQNQQETNEKDLLALDKWTNLWNWFNISNWLWYIKIFIMIIGGLIGLRIIFAVLAIVNRVRQGYSPLSFQTLIPNPTEADRPGGIEEGGGEQGRTRSIRLVNGFLALAWDDLRSLCLFSYHRLRDFVLIAARTVGTLGLRGWEILKYLVNLVWYWGQELKNSAISLLNTTAIAVAEGTDRIIEIAQRAFRAILHIPRRIRQGLERALL

SEQ ID NO: 26 - protein, 204 aa, human immunodeficiency virus nef protein
  VARIANT 132: Xaa = Any Amino Acid
MGNKWSKSWPQVRDRMRRAAPAPAADGVGAVSQDLAKHGAITSSNTAATNDDCAWLEAQTEEEVGFPVRPQVPLRPMTYKGAFDLSFFLKEKGGLDGLIYSKKRQEILDLWVHNTQGYFPDWQNYTPGPGTXYPLTFGWCFKLVPVDPSEVEEANEGENNCLLHPACQHGIEDEEREVLKWKFDSSLARRHIARELHPEFYKDC

SEQ ID NO: 27 - protein, 158 aa, human papilloma virus E6 protein
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHKIAGHYRGQCHSCCNRARQERLQRRRETQV

SEQ ID NO: 28 - protein, 105 aa, human papilloma virus E7 protein
MHGPKATLQDIVLHLEPQNEIPVDLLCHEQLSDSEEENDEIDGVNHQHLPARRAEPQRHTMLCMCCKCEARIELVVESSADDLRAFQQLFLKTLSFVCPWCASQQ

SEQ ID NO: 29 - DNA, 843 bp, human papilloma virus
cggtgtatataaaagatgtgagaaacgcaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcattcaaagatttatttgtggtgtatagagacagtataccgcatgctgcatgccataaatgtatagatttttattctagaattagagaattaagacattattcagactctgtgtatggagacacattagaaaaactaactaacactgggttatacaatttattaataaggtgcctgcggtgccagaaaccgttgaatccagcagaaaaacttagacaccttaatgaaaaacgacgattccacaaaatagctgggcactatagaggccagtgccattcgtgctgcaaccgagcacgacaggagagactccaacgacgcagagaaacacaagtataatattaagtatgcatggacctaaggcaacattgcaagacattgtattgcatttagagcctcaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagctagaattgagctagtagtagaaagctcagcagacgaccttcgagcattccagcagctgtttctgaaaaccctgtcctttgtgtgtccgtggtgtgcatcccagcagtaag

SEQ ID NO: 30 - DNA, 11835 bp, Epstein Barr virus
ggccgctgttcacctaaagtgacgcaaggtctgtcagccgccagggtccgtttaccaggctttcaggtgtggaatttagatagagtgggtgtgtgctcttgtttaattacaccaagatcaccaccctctatccatatcccacaattgataaacctccgcatgtccaaccaccacgttgaacaggatgtggcaccctaagaggacgcaggcatacaaggttattacccagtccttgtatgcctggtgtccccttagtgggacgcaggcctaggtagcatcatttacactaaaagcagtgaccttgttggtactttaaggttggtccaatccataggctttttttgtgaaaacccggggatcggactagccttagagtaactcaaggccaagcatttcacacctgcaaatgcaccatgtaaccacagatctaaactgaaagttgcagctttagatggcaaggaaacttgggtttcaggcatagaaagcctggctcactatagcagcccatgtttgttccagggtgggggaaaggcacgtgcccttagaaaacttagctgcaaaaattctattgtgttgggagagcctctatatctaaaggcctttcctcacaatacaaatgttactaacgtctgccctctggagacctgctatgtggctagacgtatggcctacccaagacgttgggggtctcgggtaggccatgattcttccaggcataggttacaaccagtcactgctatcaagcctactcagttcccaacgcagcacataccccccgcctctcctgccatgaggacttatggcagtgtttactgttctgcttttactcttggaccaggctgtcattctatcagaataacaggggaagcaaggccccctgcttcagcgggacacgtgtttctagaatctcggagccaataactacctgcccctctaatctgtatgctgcatgaaaaaccacatacacgtgatgtaagtttagccagtttattgttacaccaatgccccgaaagtctccccctgtccctttgggtctcaggacccagccctggagctcgggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggatgggggtgcgctcccaggccggaccctggtgccaggcagggaccccgcgccacccgcttcatggggggggaggccgccgcaaggacgccgggccggctgggaggtgtgcaccccccgagcgtctggacgacgctggcgagccgggccagctcgccttcttttatcctctttttggggtctctgtgcaataccttaaggtttgctcaggagtggggggcttctcattggttaattcaggtgtgtgattttagcccgttgggttacattaaggtgtgtaaccaggtgggtggtacctggaggtcattctattgggataacgagaggaggaggggctagaggcccgcgagatttggggtaggcggagcctcaggagggtcccctccatagggttgaaccaggagggggaggatcgggctccgccccgatatacctagtgggtggagcctagaggtaggtatccatagggttccattatcctggaggtatcctaagctccgcccctatataccaggtgggtggagctaggtaggattcagctaggttcctactggggtacccccctaccctaccttaaggtgcgccacccttcctccttccgttttaatggtagaataacctataggttattaacctagtggtggaatagggtattgcagctgggtatatacctataggtatatagaacctagaggaagggaaccctatagtgtaatccctcccccccctacccccccctcccttacggttgcctgagcccatcccccaccccagcaccccggggtgacgtggcaccccgcgtgccttactgacttgtcacctttgcacatttggtcagctgaccgatgctcgccacttcctgggtcatgacctggcctgtgccttgtcccatggacaatgtccctccagcgtggtggctgcctttgggatgcatcactttgagccactaagcccccgttgctcgccttgcctgcctcaccatgacacactaagcccctgctaatccatgagccccgcctttaggaagcaccacgtcccggggacggaagctggattttggccagtcttcaattttggggagtggttttgtgtgagccggaagttggcaatggggtgagggtggcgctggttaagctgacgacctcccaaggtctctcaccctgggtacacaggtggggcggcagcctctaactttggctgtggcctctatttcctccctttcctagccagggccatgtgttcctgcatgtctacttgcctcctgtggtggcagagcttggccctgggcccaacccccgccttgggagcctgtaggggccaacacccttggtttgtttgtgttcctgtttgctggcaacttactggcagccgagcagattctaatgggcgcccgccttctttctctcttgttttattaatagaatctcagccaggacctatacctgagacttcaaagtctggtcctgggttctgagacccccaagatttgtcatgcacacctgcacacctgttggtattgggtttctattcttgagtgtgaaagtttgtaaaaaaattcataaaatgtcactaattcctcttacctgtttagggtattgtgcaattcttcagcctgcctattttcaatttgcctaaggtggcaatttaagatgtggttaattaaccattttcctgtctgacaccactgcatgggcaaccgggttccatggcacatttagagataaacatagatgtcttgtcttgctcatgtgcagaggagggggtgttggtgtgcaatatagtttctggattccaaattgagttgggggtgctattttcactatggaattaaattactgacattagacagtggacaccgggctatatgtggggatgtctgtggcttgtcatttcctcttagaaggtaatcccccatcttaacttccctttaaattgtgatgcaagccctgggttatttatagaatgattatctaggtttgatagtctgaaggctgggcagagaatgtttgtaatttttattcaccttctttaccccccacgagtatccagttctagaagatctcctgatatcccgggctgccattattcccttgagtgttatagcttcctcttaacttaagcaagagctccaggatgttagcttttttggtggggctggttgtcaggaagaggttccagtgttgtcctttatttttagatgttagctttgtgttaggttagtatgggctgggtattcactagtgaaggcaactaacacagttagacgtgctagttgtgcccactggtgtttatccggtcccaaatgtcaccacagaacacagggggctggatttggcagcagcacttgtgcttttgttgatttttacccgtgtatcagagtgggggatgctagccaatttagcttcccctccccttaacagggggtctcgcggggtgccaattgtcgcctgccttcccccgcttccccttgttaacttatagcatgataggtaggtcacctaacgtggaagcctggtgggtgatccttcctcggtagggagcgcttagggctgttgagctcaacagccccacctgggtaaaatgtatgttctaaagagttacccaattataacaaaactgttgtagggtaacgaagacctgatggaagtggtattgttgccgttgaaagacgggtgtcctggctcaagttcgcacttcctatacagtgttaaagccttgtatcggaagtttgggcttcgtcccagtgtactcgataatgtcgactgctgcgaaaggtttggaccgtcttccagtaggtgttgggggtcccaaatcacgaggttaggcaggtgcacttggctctttaggagggacccttaagccagacaatgtagtgccccttttttttgcaaattggccttattattaatttcttgttaacactaattctgttctatgaccctgtgtttttcagatgccgttgaacgtgtcactgagctgaatttggacgcagctacttgacctttgcccccgtgcctccagcgctgataagtgctgcgtccactttgtgttacaggtgggccaaacctccagaatatcaattggtggggccttggtgggctgcataaggcagtaggtttgaggtgacctacttggaccatgtggatccagtgtcctgatcctggaccttgactatgaaacaattctaaaaaaatgcatcatagtccagtgtccagggacagtgcactcggaagtctcatcatctccgtttgtgtgtttagtgtggccagtacggccacccctgtgccacgccctggcatgctgctgacatctggccgccaatttcagcgggcccttttcccccttgttcaccccatagcaagaagggtaggttacatgggtattttcccatcagcacctgactggccggtgcaattagaggagagggcaacaacgcaaggctgttgttttatttgggttacaagagctgcggcggtcgatgggttcactgattacggtttcctagattgtacagatgaactagaactgtcacaatctatggggtcgtagacagtgtgcttaccagacttccatggaagatgtgaatttgctgctagctatatgggtggtgctatgggctccctagggactcatgtagtggggctttgtgatagctaatgaatgtggcagctgttgtttgtactggaccctgaattggaaacagtaacttggattctgtaacacttcatgggtcccgtagtgacaactatgctgaatatcttgaatatgggaggaggggggctttgggttccattgtgtgccctttcctggccaacgtgagggtcctagtgttatagggcgtggcagttttcttgagggctaataacccgggtgaggcggttgtcacaggtgctagaccctggagttgaaccagtaccactcggttacaaagtcatggtctagtagttgtgaccctgcaaagctacgtggggatgagcagccagggactttggttggcaagcagacaggcggcgcattggaaccccagaggagtgtcccggggccacctctttggttctgtacatattttgttattgtacataaccatggagttggctgtggtgcactccatctggtaagggggctggtgcggacgcctgtgtttagtctatgccaatgtttacctgccttgggttactattccaaacgaccacacctttgaggacacctggagccctgatcattctcggcttttactgccacctggcttctgttgggtcagacagtttggtgcgctagttgtgtgcttagcagcaacgcacaccaggctgactgccttagcagtgtggccctttattgtggcatcctaaggagggattctggagtgcctttcgcgtgaagcatgccctgagacgtactcgagttaggacttaatcgctcctgtgccgctggatgagggagcgccaatttgtacatcctagctctggccatagagttagcccacccttgtgtctccctttggcctttgcggtgccaatttccggtggtttcccttttccgcccgtttatccaatagcatgtaagagaggttgcctagatttggcaactttgagggaacgttccgtgtagctggtgacctaacacccgcccatcaccaccggacagattctgaacttgtcctgtggtgtttggtgtggttttggggtacgcaggagtacgttggaatgctttggagccgagagggatgggcccgcttgtgcgcttatgtgttacacggtgccaataaccggcccggtgcggctgccccgtgacccgtgggccttaccttcctggccatcgggggaccctggtgctagggtcccttgtgttgctttctgccataggggggaaagcatcgccttcagaattggctgctccgttggaacatttgaggcctactgtatccgtgtcctgacaacattccccgcaaacatgacatgggttaatttaaacatgttttgtttgcttgggaatgctcttagggcctggaagcttgtcattggattcatcgtttcctgaactacaggcgtagggcctattgtagcaggcatgtcttcattcctgcgtaccgaatggcatgaaggcacagcctgttaccattggcaccttttttccatgtaaacctccgtgatcctgggtcctttggagactcaagtgtgaatttgttttggtgttcggcgccagggtatctcgacgttggaatgtcaactcaacttgggcacctcgataaccggctcgtggctcgtacagacgattgtttggctctgtaacttgccagggacggctgacgatgtgtttagtctgccacttgcatccggcgctttggttactcgggagactaatggggggtgtggtatggcacaggctgggggtgagtctggggatgtccctgggcgttgctgcagcccattcgccctctggggatgagatgttcaggggtggccggtaccctacgctgccgatttacataatataaattgtaaatgctgcagtagtagggatctggacgcgcgacctgctactcttcggaaacgccaacccaggagcgtcgcctctggccccatactcccgccatgcgactgctcgccccctcccaggcctccctggtgagcccttgccgctccccgcattcctgctttcggcgcccctgcggatcccgatgacagcaggcctttccttcccccgttaatgaaaagaatgacagtgaggttgtgacagaaggacagctttattcagtttacagagtgccctcggaggctacgatattcccgttaaatgtcttgttgattctctcaaaggtggggagggaggagctctccacaacaatgttccctggcagcgtgagcgcgcagccctgccgttggatgtatcttctcatgatggtgctgatagaggggtctccggcgtagatgaaaaaggcctgggccatgctctggccggtcacgatcgttatggggttgttggaaatgttccggaccgtcagcttgagggtctggcccggcttccactcctgtgggtagacgtagaagaccgggttggaggagtgggacacgacaacggccgtaatcttggagctcaggggggcctcgtaggtgttgttgtattccagctccgtgatgaaattaggaggaataatcacaggggagccaaagtagcggatgtctgtggattccccgtcccagcgccagtggctcttagggtaggggttgtaacggaaggcaataatcacatcatccaatagggtcatgcccaccttgacgttcagcgggccctctcgtttcaggtccggcgtgtccacggagactcggacgtagcccttaccgcggcgtatggcgtttaccggacacaccttccccgggaatgtgtgaatacgggcgtatgactttagaaatgggggcgtgtgctgcgccagcaggtaaggcaggcactcgtcctggctggtgacgggagagccactgaggaagatctggggctcgctggtgtttagcttgtccccgctctgggtgcaggagcgtgtcagctgaatgtcgctctgcccgggcagaatctgcaggtagaggtaggggttcttgaccaatctgatgggcacaatgtaccaggtaaacttccctttctctatgaacaggctgcgcggattcaggacgcttagcacgatgtcctggtcagagtgcataacgaagaagggcttgaggaatacctcgttgtcttccgctccaaagaacaaaaacgcgaccgtaaagtagcggctgccgtaggtggtcgtgttgaaggagaaagaaggtaacttgaagctgagtatctggcccaccgaggggcagggaggcagctcttggcactgcgcgtccagctgcaatacctgcttgttggtgacgcggacgtatgaggggaagatctcgtacttccacacgcctctcatgaacgacgtgtctggtttttcagtgggccgcaggcggcggaggctgttcctgaacgacgagcgccgggacgctagtgctgcatgggctcctccggggtaagcttcggccatggccggagctcgtcgacgggcaaggtgagagtcggggggcgggcgacggtgcggccccaatacaactctccgctcgttagctggtagaatatccgcccggcgtctaggttgtcacttcgctcggccggccagaagagcgcaagtccaagtctggtgctggggccgatgtgcagcggtttgtgcccgcagttgtagactgtcatttttatgggcgagtgggcggtccacacgcgcgggcgcagcacccattggtcgcacgccgcctcctggaatgtaaacccccagagagagggcgtgccgccctggagatggccctgtgccatcacatgtatttcctccttgggtggaacaacggcgtcgtgctccgggtggagggggaatagcgtccaggcatctttcagggtcacgagaccggggtccatgctcagagaacagccctcccgggcggtgggcggcccgggctccagcagaacgtcgcagacccagccctcctcggccctgtccacctgtatgtccaggtgcacggacccggaggctgcgtctcgtgacatggccaggcctggtgccagccgaccacgtcccgtgtcccagccgaggccgcgccagagcagagcccgggactgactcagggccacatcccctcggcccgcggacgccgcctcgccagcccccgggccttcatgggcccgctttctacctctctccggcaccccagcctggtcagccgcagaggaagcatgaccttggggtgggacggggcaggcgtgatcctgggcgcaatctttgccgatccccacaccttcactccttgttaggttgatagaatgtcggtaccacgccacggggggcgggcccgcatagggaaaagccagggagagcgatgtgggcgaggatgggctcaggcggccccagacacgcaatttgcccccctgggcggccgcagcctgcccctcggcggcccgtgccccagctccgtcacggggggcgcataggaggggtatatctaggatagccgcacctacacaaatgagacacagacacaggtcgtgaggatttaggcaacgcaggcttgtctttatagttacaaacatgggagcgtgcacctggaagatgcagctggggtagatctttacatctttacagggcgcagcggccgccagacactgaagggcagagttcacggcgggcacctcccagagggagcccaccagcccgtacctggccacggcc

SEQ ID NO: 31 - DNA, 2337 bp, Plasmodium falciparum antigen
aaaaaagaaaattataaataaatatatatattcgtgtaaaaataagtagaaaccacgtatattataaattacaattcatgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataataataatggagataatggtcgtgaaggtaaagatgaagataaaagagatggaaataacgaagacaacgagaaattaaggaaaccaaaacataaaaaattaaagcaaccaggggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaatcctaatgcaaacccaaatgcaaatcctaatgcaaacccaaatgcaaacccaaacgtagatcctaatgcaaatccaaatgcaaacccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacaatgctgtaaaaaataataataacgaagaaccaagtgataagcacatagaacaatatttaaagaaaataaaaaattctatttcaactgaatggtccccatgtagtgtaacttgtggaaatggtattcaagttagaataaagcctggctctgctaataaacctaaagacgaattagattatgaaaatgatattgaaaaaaaaatttgtaaaatggaaaaatgttccagtgtgtttaatgtcgtaaatagttcaataggattaataatggtattatccttcttgttccttaattagataaagaacacatcttagtttgagttgtacaatatttataaaaatatatactactttttttcttaattttcatttttctttatattttcctatttaatttatttttttgtgaatatttaattacgtttgcgattaattgtagaaatatatatgtatatactatatttatagaatgtgttattctcaaaaacaacaacaaaaaaaaaaaaaaaaaaaaaaaaaagaaaaaaggattaaaagtaaaatagttataaatattttcaaaaatatttataacacaaaaaatacttcgaagttcatttaacatttttgtttatttatttatttatatatttcatttttacgtatttatattataaaatggtgtatcttaaaaatagtgaactatatatataaaatattaatttaaaaaaattataactttctttttattttctaaaataacttaaaaattatatgtttaagaaaggggtaaattataatatttgtataaatatataaacatagatatattaaataaaataacaaatgtactatatttgtgcataagacgtatacgctttatataatacaacaatattaattgtaataatatttgtggtagtgtgaacactaaaattgataataatgattataatacagaagaaataaaaaatgaatccaatataggatttacaacaaatattcatgaagcaaaaataattcaagaaaagacatatggattaataataaacgataaaataaagaaagaagaatatgatgattgtaataataataataataataatattataatacagataagagaagttggacttaattattttggagatactctcgatgaatcgaatccatgtaatgatcttacaggtattaatatatgggaaagttgtcttgtggctagtcgatggtttagcgatttatctttacagaattttttttcgaataaaaatattttagaaattggtgctggcagtggtttggctagtataataatatttatatattctaatatttacaa

SEQ ID NO: 32 - DNA, 729 bp, Plasmodium falciparum antigen
cgaaaaatatttaattatctaaataaatttaattaaaaatttttataacatattttatttaagattttataataattaagttttaatttcttttgatccaaagtttttaataattaaatttgtagatttttaatttatttaatatattcaaaatgaaaatcttatcagtattttttcttgctcttttctttatcattttcaataaagaatccttagccgaaaaaacaaacaaaggaactggaagtggtgttagcagcaaaaaaaaaaataaaaaaggatcaggtgaaccattaatagatgtacacgatttaatatctgatatgatcaaaaaagaagaagaacttgttgaagttaacaaaagaaaatccaaatataaacttgccacttcagtacttgcaggtttattaggtgtagtatccaccgtattattaggaggtgttggtttagtattatacaatactgaaaaaggaagacacccattcaaaataggatcaagcgacccagctgataatgctaacccagatgctgattctgaatccaatggagaaccaaatgcagacccacaagttacagctcaagatgttacaccagagcaaccacaaggtgacgacaacaacctcgtaagtggccctgaacactaaacagctgtaaacttttttgttaatgggtttttttgaaacacgtgaaaataatttttatttatgattatattatatatattgctatttt

SEQ ID NO: 33 - DNA, 594 bp, homo sapiens survivin
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgagagagctctgttagcagaatgaaaaaattggaagccagattcagggagggactggaagcaaaagaatttctgttcgaggaagagcctgatgtttgccagggtctgtttaactggacatgaagaggaaggctctggactttcctccaggagtttcaggagaaaggcaaaggaaaccaacaataagaagaaagaatttgaggaaactgcggagaaagtgcgccgtgccatcgagcagctggctgccatggattga

SEQ ID NO: 34 - DNA, 1605 bp, homo sapiens survivin
gtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttcaaacacaaaaaaaaaaaaaaa

SEQ ID NO: 35 - DNA, 3107 bp, homo sapiens melanoma antigen
atgatacagtccaagcacctggatgatgagtatgagagcagcgaggaggagagagagactcccgcggtcccacccacctggagagcatcacagccctcattgacggtgcgggctcagttggcccctcggcccccgatggccccgaggtcccagataccctcaaggcacgtactgtgcctgcccccccgcaacgtgacccttctgcaggagagggcaaataagttggtgaaatacctgatgattaaggactacaagaagatccccatcaagcgcgcagacatgctgaaggatgtcatcagagaatatgatgaacatttccctgagatcattgaacgagcaacgtacaccctggaaaaggtgggtgcaggatgggagcagctctgtgggggaagagcgggcatgggggtgcggtgaccctgcagcccctcaaggcccagtctctggagccatctctcacctctccgactctgagcttccactgcactggcagtttgactcgtgcttcctgccctcggcttctgtctctcatgctctctgagtgtctcgccgtctggccaggtgggtctcatcgcctctgccagcgtcagctcccacagcgaaggtcttccgtgtgctgtcttcttctgccctcgctcacgagtttggattccttgctgaggagcagttctaacccggaatcactgtctgccggcaggatgcccagcatggggtttggatctcacactctgttttctcccccacgtagaagtttgggatccacctgaaggagatcgacaaggaagaacacctgtatattcttgtctgcacacgggactcctcagctcgcctccttggaaaaaccaaggacactcccaggctgagtctcctcttggtgattctgggcgtcatcttcatgaatggcaaccgtgccagcgaggctgtcctctgggaggcactacgcaagatgggactgcgccctgggtatgattggcctctccagctcctcccctcggtgctatcctctggccaaagaggtcctgggattgcaatagcctggtggtctggcgcaagggcgtggggtgccctgggctcggtagagagcaaaggatctcaccagggcggatggggaagcggtgctggacgctgctcagccctctctctgctctgtggccccagatgacatctaagagagacagtcagagtcagggattccatcaaatccctacctggggcgcccctgaccaacagtcctctggcctctgctgcatgcccaggcctccacagcgactccccgggggctgggaagtcatagtcatgctagggagggcccctgccaccgtctctgctcatggattcctttccttgccctcagggtgaggcacccattcctcggcgatctgaggaagctcatcacagatgactttgtgaagcagaagtaagtatcacctgagctaactgcggctctcactcgagcatcctttgtgtgctggtctggctgagaaagcagttccctatcccaaatcttcaactggagggatgggtgcctctgacctgggagtgagtggcagtggggggtatgcgagtgtgtggggagccgaaggccagggcggtcttgggaaaagggagctcacgtcacctgagaacacggtgtggggtgtgaaaacggccgccatcaccttgagcacctgccctgtagactgacacaagagttccccctggtttacacctaaggaacccggagctcagagaggagacgcctctgagcatggctcccagctggtaagggcctcagcccaactctcctgattttcaggccaggggccaccctctccccgtccctggaggacttgccaacgcacaggcgcgcatgcacaccaacaaagggtcaggacttgaggaggatgcctggagcacgcttctcctggctgactgtttcttcctctccagtcgtttcctctggtgggcctctccagggctccgccggggtgtggccaagaccctcgaggtggggtgtgctcagagcagggggcctgaagaatggctcctctgtttacaacacacccaacaggaagctggggtcatcgtgatgaggggcacaaacttgtggcctccctacagacaaatgccctacatgtggaccccctgcacctccgcatggcttccggggaggaccaatggcaaaaggctttgaaggcctcacttttgcaggcagaagtcctgggagtgggtttgggaatgagtgaagggctggaggggcaggacagtcctcttccaggagctgagctgcggcatcgggttgaggaggggccccctggaacccatccgttcagcaacaggtctgcttggctagcagcaaagtttactttcctctcatgccaaggtacctggaatacaagaagatccccaacagcaacccacctgagtatgaattcctctggggcctgcgagcccgccatgagaccagcaagatgagggtcctgagattcatcgcccagaatcagaaccgagacccccgggaatggaaggctcatttcttggaggctgtggatgatgctttcaagacaatggatgtggatatggccgaggaacatgccagggcccagatgagggcccagatgaatatcggggatgaagcgctgattggacggtggagctgggatgacatacaagtcgagctcctgacctgggatgaggacggagattttggcgatgcctgggccaggatcccctttgctttctgggccagataccatcagtacattctgaatagcaaccgtgccaacaggagggccagcttcttctcctggatccagtaagagtttcggcaccgttgacgaactgcagcgatcttactggccaagccagagcgcctcctctcagattccttctcgacacagcaccctaggcggcttcttcctgtcagtcggaggtggcatgcaagatgaagctctctttgctcttcccgctttcattttgtgcttttccttgtgttttcatgttttgggtatcagtgttacattaaagttgcaaaattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaag

SEQ ID NO: 36 - DNA, 2509 bp, homo sapiens melanoma antigen
ccccactgtctgccccgcaggctgtgcactgcccttccatcatctggcaggcccccaaaggtcagcccccggtgccacacgagattccaacgtcaatggaattccaggaggtgcagcagacacaggcgctggcctggcaggcccagaaggcccccactcacatctggcagcccctgcctgcccaggaggcccagaggcaggctccccccttggtccagctggagcagccctttcagggagccccgccctcccaaaaagccgtgcaaatccagctacccccccagcaggcccaggcatcgggtccgcaagcggaggtgcccacactgccgctccagccttcctggcaggcaccgcctgcagtcttgcaggcccagcccggacccccggtagcagcggcaaattttcccctgggctccgctaaatcattgatgactccatcaggagaatgcagggcctcttctatagaccgcaggggctcctctaaagagcgcaggacctcctcgaaggagcgcagggccccttcaaaagaccgcatgatctttgctgccaccttctgtgctcccaaggcagtgtcagctgcgcgagcacacctgccagctgcctggaaaaacctgcctgccacaccggagacctttgctccctcctcaagtgtcttcccagctacctcccagtttcagcctgcctctctgaatgcctttaaaggcccctctgctgcctcagagaccccaaagtcactgccatatgctctgcaggatccctttgcctgtgtagaggccctgcctgcagttccatgggtcccacagcccaatatgaatgcctcaaaggcatcgcaggcagtgcccaccttcctgatggctacagcagctgccccccaggcaactgccaccactcaagaggcctccaagacctccgtcgagccgccacgccgctccggcaaggccacccggaagaagaagcatctggaagcccaagaggacagccgtggccacacgctagcctttcatgactggcagggcccaaggccctgggagaatctaaatctgagtgactgggaggtccaaagccctatccaggtctcgggtgactgggagcacccaaacaccccccgtggcctgagtggttgggagggccctagcacctccaggatcctgagtggctgggaagggcccagcgcatcctgggccctgagtgcctgggagggcccgagcacctccagggccctgggtctctctgaaagcccagggagctctctgcccgtagttgtgtctgaggtcgcaagtgtctctccgggatccagtgccacccaggataattccaaggtggaggcacagcccttgtctcccttggatgagagggcaaatgcgttggtgcagttcctcttagtcaaggaccaagccaaggtgcctgtccagcgctcggagatggtgaaagtcatcctccgagagtataaagatgagtgcttagatatcatcaaccgtgccaacaataagctggagtgtgcctttggttatcaattgaaagaaattgataccaaaaaccacgcctatattatcatcaacaagctgggctaccatacagggaatttggtggcatcctatttagacaggcccaagtttggccttctgatggtggtcttgagcctcatctttatgaaaggcaactgtgtcagggaggatctgatctttaattttctgttcaagttagggttggatgtccgggagacaaacggtctctttggaaatactaagaagctcatcaccgaagtgtttgtcaggcagaagtacctagagtacaggcgaatcccttacactgagcccgcagagtatgagttcctctggggccctcgagcattcctggaaaccagcaagatgcttgtcctgaggtttttggccaagctccataagaaagatccacagagctggccattccattaccttgaagcgctcgcagagtgtgagtgggaagacacagatgaggatgaacctgacaccggtgacagtgcccacggccccaccagcaggccccctccccgctaataggtgtagcagagatctcgctcctgtgtttccctggccagaggccactgacagggtggggggacatttttgttcctggtgtttgtgttccagttccacgagtgtaagtttggattttcaacttggtttcgtatctgccaaagctttgtacattttttatgtggtgttgatttcaatcggctactgttctgttctgtattttggcatctgtgtttttaagtgagatctgtggttctctgttttgtgttataattgttatgttttggtatcagctttgtgctggctttgtgaaatgaattgagaagctatccatctcatttctggtatagttcatgtagcattgtaatcggttgttctttgaacgttcaaatgactcatcagtaaaaactgtctacagagaagtaaatatctatatctatatatataaatatactttcagcataaaaaaaaaaaaaaaaaaaaaaaaaaaaaaa

SEQ ID NO: 37 - DNA, 1782 bp, homo sapiens melanoma antigen
agtgttgcaactgggcctggcatgtttcagtgtggtgtccagcagtgtctcccactccttgtgaagtctgaggttgcaaaaggactgtgatcatatgaagatcatccaggagtacaactcgaaattctcagaaaacaggaccttgatgtgagaggagcaggttcaggtaaacaaagggtgccacatctcctgcctttctgctcactttcctgcctgttttgcctgaccacagccatcatgcctcggggtcagaagagtaagctccgtgctcgtgagaaacgccgcaaggcgcgagaggagacccagggtctcaaggttgctcacgccactgcagcagagaaagaggagtgcccctcctcctctcctgttttaggggatactcccacaagctcccctgctgctggcattccccagaagcctcagggagctccacccaccaccactgctgctgcagctgtgtcatgtaccgaatctgacgaaggtgccaaatgccaaggtgaggaaaatgcaagtttctcccaggccacaacatccactgagagctcagtcaaagatcctgtagcctgggaggcaggaatgctgatgcacttcattctacgtaagtataaaatgagagagcccattatgaaggcagatatgctgaaggttgttgatgaaaagtacaaggatcacttcactgagatcctcaatggagcctctcgccgcttggagctcgtctttggccttgatttgaaggaagacaaccctagtggccacacctacaccctcgtcagtaagctaaacctcaccaatgatggaaacctgagcaatgattgggactttcccaggaatgggcttctgatgcctctcctgggtgtgatcttcttaaagggcaactctgccaccgaggaagagatctggaaattcatgaatgtgttgggagcctatgatggagaggagcacttaatctatggggaaccccgtaagttcatcacccaagatctggtgcaggaaaaatatctgaagtacgagcaggtgcccaacagtgatcccccacgctatcaattcctatggggtccgagagcctatgctgaaaccaccaagatgaaagtcctcgagtttttggccaagatgaatggtgccactccccgtgacttcccatcccattatgaagaggctttgagagatgaggaagagagagcccaagtccgatccagtgttagagccaggcgtcgcactactgccacgacttttagagcgcgttctagagccccattcagcaggtcctcccaccccatgtgagaactcaggcagattgttcactttgtttttgtggcaagatgccaaccttttgaagtagtgagcagccaagatatggctagagagatcatcatatatatctcctttgtgttcctgttaaacattagtatctttcaagtgtttttcttttaatagaatgtttatttagagttgggatctatgtctatgagcgacatggatcacacatttattggtgctgccagctttaagcataagagttttgatattctatatttttcaaatccttgaatcttttttgggttgaagaagaagaaagcatagctttagaatagagattttctcagaaatgtgtgaaagaacctcacacaacataattggagtcttaaaatagaggaagagtaagcaaagcatgtcaagtttttgttttctgcattcagttttgtttttgtaaaatccaaagatacatacctggttgtttttagccttttcaagaatgcagataaaataaatagtaataaatta

(19) United States
(12) Patent Application Publication
(10) Pub. No.: US 2004/0258688 A1
(43) Pub. Date: Dec. 23, 2004

(54) Enhanced antigen delivery and modulation of the immune response therefrom

(76) Inventors: Daniel Hawiger, Branford, CT (US); Michel Nussenzweig, New York, NY (US); Ralph M. Steinman, Westport, CT (US); Laura Bonifaz, Del Alvaro Obregon (MX)

Correspondence Address: KLAUBER & JACKSON, 411 HACKENSACK AVENUE, HACKENSACK, NJ, 07601

(21) Appl. No.: 10/800,023

(22) Filed: Mar. 12, 2004

Related U.S. Application Data
(63) Continuation-in-part of application No. 09/925,284, filed on Aug. 9, 2001.
Application No. 09/925,284 is a continuation-in-part of application No. 09/586,704, filed on Jun. 5, 2000.
Application No. 09/586,704 is a continuation of application No. PCT/US96/01383, filed on Jan. 31, 1996.
Application No. PCT/US96/01383 is a continuation of application No. 08/381,528, filed on Jan. 31, 1995, now abandoned.

(51) Int. Cl. G01N 33/53; G01N 33/567; A61K 39/395; C12N 5/08

(52) U.S. Cl. 424/144.1; 435/7.2; 435/372

(57) ABSTRACT

The present invention relates to methods for targeting antigen to antigen presenting cells through specific endocytic receptors, which results in persistent antigen presentation in the context of MHC molecules. Such highly efficient antigen presentation results in robust and long lasting immune responses, in particular cell mediated responses. The invention provides for immune compositions containing antibodies to DEC-205 in combination with the antigen for eliciting either T cell mediated immunity when delivered with a dendritic cell maturation factor, or for inducing tolerance when delivered in the absence of a dendritic cell maturation factor. The compositions described in the present invention are effective as a single dose at low concentrations and show efficacy even with non-replicating subunit vaccines.

### CROSS-REFERENCE TO RELATED APPLICATIONS

This application is a continuation-in-part of co-pending U.S. Ser. No. 09/925,284, filed Aug. 9, 2001; which is a continuation-in-part of co-pending U.S. Ser. No. 09/586,704, filed Jun. 5, 2000, which claims priority to PCT/US96/01383, filed Jan. 31, 1996 and to U.S. Ser. No. 08/381,528, filed Jan. 31, 1995, now abandoned. Applicants claim the benefit of this application under 35 U.S.C. §119 (a-d) and 35 U.S.C. §120. All of the prior applications are incorporated herein by reference in their entireties.

### FIELD OF THE INVENTION

This invention relates to novel immunogenic constructs and methods for generating efficient antigen presentation and robust immunological responses in vivo and for promoting long lasting immunity upon administration of these constructs to mammals. The invention also relates to methods for inducing tolerance to antigens for which an immune response is undesirable.

### BACKGROUND OF THE INVENTION

Dendritic cells (DCs) are inducers of primary immune responses in vitro and in vivo (J. Banchereau, R. M. Steinman, Nature 392, 245-52 (1998); C. Thery, S. Amigorena, Curr. Opin. Immunol. 13, 45-51. (2001)). In tissue culture experiments, DCs are typically two orders of magnitude more effective as antigen presenting cells (APCs) than B cells or macrophages (K. Inaba, R. M. Steinman, W. C. Van Voorhis, S. Muramatsu, Proc Natl Acad Sci USA 80, 6041-5 (1983); R. M. Steinman, B. Gutchinov, M. D. Witmer, M. C. Nussenzweig, J Exp Med 157, 613-27 (1983)).

There is indirect evidence from a number of different laboratories suggesting that DCs also may play a role in maintaining peripheral tolerance. For example, injection of mice with 33D1, a rat monoclonal antibody to an unknown DC antigen, appeared to induce T cell unresponsiveness to the rat IgG (F. D. Finkelman, A. Lees, R. Birnbaum, W. C. Gause, S.C. Morris, J Immunol 157, 1406-14. (1996)). However, the specificity of antigen delivery was uncertain and the relevant T cell responses could not be analyzed directly. In addition, peripheral tolerance to ovalbumin and hemagglutinin expressed in pancreatic islets was found to be induced by bone marrow derived antigen presenting cells (A. J. Adler, et al., J Exp Med 187, 1555-64. (1998); C. Kurts, H. Kosaka, F. R. Carbone, J. F. Miller, W. R. Heath, J Exp Med 186, 23945. (1997); D. J. Morgan, H. T. Kreuwel, L. A. Sherman, J Immunol 163, 723-7. (1999)), but the identity of these antigen presenting cells has not been determined (W. R. Heath, F. R. Carbone, Annu Rev Immunol 19, 47-64 (2001)).

For many diseases that lead to high mortality and morbidity, such as AIDS and malaria, it is likely that protective vaccines will need to elicit strong T cell mediated immunity composed of IFN-γ secreting CD4⁺ helper and CD8⁺ cytolytic T lymphocytes (Seder, R. A., and J. R. Mascola, (2003), Basic immunology of vaccine development. Academic Press, Boston, pp 51-72; McMichael, A. J., and T. Hanke, (2003), Nat. Med. 9:874-880; Reed, S. G., and A. Campos-Neto, (2003), Curr. Opin. Immunol. 15:456460; Finn, O. J. (2003), Nat. Rev. Immunol. 3:630-641). To induce such responses, it would be valuable to harness the dendritic cell (DC) system of antigen presenting cells (Lu, W., X. Wu, Y. Lu, W. Guo, and J. M. Andrieu, (2003). Nat. Med. 9:27-32; Steinman, R. M., and M. Pope. (2002), J. Clin. Invest. 109:1519-1526). At least 3 sets of DC functions are pertinent. DCs efficiently process antigens, including complex microbes and tumor cells, and display these on both MHC class I and II products to CD8⁺ and CD4⁺ T cells respectively (Jung, S., D. Unutmaz, P. Wong, G.-I. Sano, K. De los Santos, T. Sparwasser, S. Wu, S. Vuthoori, K. Ko, F. Zavala, E. G. Pamer, D. R. Littman, and R. A. Lang. (2002), Immunity 17:211-220; Thery, C., and S. Amigorena. (2001), Curr. Opin. Immunol. 13:145-51). DCs undergo a complex differentiation or maturation program in response to a panel of stimuli including microbial ligands for Toll Like Receptors (Janeway, C. A., Jr., and R. Medzhitov. (2002), Annu. Rev. Immunol. 20:197-216; Takeda, K., T. Kaisho, and S. Akira. (2003), Annu. Rev. Immunol. 21:335-376), innate lymphocytes (Bendelac, A., and R. Medzhitov. (2002), J. Exp. Med. 195:F19-23; Fujii, S., K. Shimizu, C. Smith, L. Bonifaz, and R. M. Steinman. (2003), J. Exp. Med. 198:267-279), and CD40 ligation (Caux, C., C. Massacrier, B. Vanberyliet, B. Dubois, C. Van Kooten, I. Durand, and J. Banchereau. (1994), J. Exp. Med. 180:1263-1272).

Additionally, DCs localize to the T cell areas of lymphoid organs (Witmer, M. D., and R. M. Steinman. (1984), Am. J. Anat. 170:465481; Austyn, J. M., J. W. Kupiec-Weglinski, D. F. Hankins, and P. J. Morris. (1988), J. Exp. Med. 167:646-651), where they select antigen-specific T cells (Ingulli, E., A. Mondino, A. Khoruts, and M. K. Jenkins. (1997), J. Exp. Med. 185:2133-2141; Bousso, P., and E. Robey. (2003), Nat. Immunol. 4:579-585; von Andrian, U. H., and T. R. Mempel. (2003), Nat. Rev. Immunol. 3:867-878), which is followed by clonal expansion.

Co-pending application Ser. No. 09/586,704 describes the endocytic cell membrane receptor DEC-205, which is present on mammalian dendritic cells as well as on certain other cell types, and describes its role in antigen processing, and exploiting the existence of DEC-205 primarily on dendritic cells for targeting antigens for uptake and presentation by dendritic cells. The application describes ligands of DEC-205, such as antibodies, carbohydrates as well as other DEC-205-binding agents for targeting antigens to DEC-205 and thus specifically to dendritic cells.

Co-pending application Ser. No. 09/925,284 describes methods for enhancing the delivery of preselected antigens to an endocytic receptor on antigen presenting cells, such as dendritic cells. Particular embodiments of the invention provide for DEC-205 as being the endocytic receptor combined with a means for modulation of the immune response such that either enhancement of the immune response or tolerance to a preselected antigen may be obtained.

It is toward novel immunogenic constructs and enhancement of highly efficient antigen presentation and immune responses that the present invention is directed. In particular, constructs are provided which serve as potent vaccines for eliciting robust and long lasting cellular and humoral immunity.

The citation of any reference herein should not be deemed as an admission that such reference is available as prior art to the instant invention.

### SUMMARY OF THE INVENTION

In its broadest aspect, the present invention is directed to methods of promoting efficient, vigorous and long lasting antigen presentation by targeting a preselected antigen to an endocytic receptor on an antigen-presenting cell. A non-limiting but preferred antigen-presenting cell is a dendritic cell (DC). Non-limiting examples of dendritic cell endocytic receptors include DEC-205, the asialoglycoprotein receptor, the Fcγ receptor, the macrophage mannose receptor, and Langerin. A preferred receptor is DEC-205. Enhanced processing and presentation of antigen to T cells is achieved by the foregoing method. The foregoing enhanced presentation by the method of the invention, in combination with other factors or conditions, may lead to a more robust immune response to the preselected antigen, or tolerance to the preselected antigen.

The foregoing enhanced antigen presentation in combination with manipulating the antigen-presenting cell may be carried out in order to modulate the immune response to the preselected antigen delivered via the endocytic receptor. To enhance the development of a cellular or humoral immune response to the preselected antigen, delivery of the antigen via the endocytic receptor to a dendritic cell (DC) in combination with DC maturation is carried out. DC maturation may be induced by any means, such as by way of non-limiting examples, CD40 ligation, such as with an anti-CD40 antibody, an inflammatory cytokine, CpG, ligation of the IL-1, TNF or TOLL receptors, or activation of an intracellular pathway such as TRAF-6 or NF-κB. In a preferred but non-limiting embodiment, DC maturation is achieved by CD40 ligation.

To induce tolerance to the preselected antigen, antigen delivery to a dendritic cell is carried out in the absence of DC maturation, such as the absence of CD40 ligation, or in the absence of any other DC maturation signal such as but not limited to those described above.

The foregoing methods are carried out in an animal in which either an enhanced immune response is desired or a tolerizing immune response is desired, or it may be carried out ex vivo and antigen-presenting cells introduced into the animal. The antigen delivery may be carried out ex vivo, using antigen-presenting cells isolated from the animal, after which the cells may be optionally isolated and returned to the animal. Subsequently, in vivo manipulation of DC maturation, such as by CD40 ligation, is carried out to direct the immune response to the desired outcome. Alternatively, both the antigen exposure and DC maturation or inhibition of DC maturation may be carried out ex vivo before optional isolation of antigen-presenting cells and introduction into the animal. Furthermore, both antigen delivery and manipulation of DC maturation may be carried out in vivo.

In addition, the methods may be carried out through use of a genetically modified anti- DEC-205 antibody or fragments thereof, whereby the amino acid sequence for the antigen is on the same polypeptide chain, either the light or heavy chain, of the anti-DEC-205 antibody. The enhanced antigen presentation and subsequent immune response is achieved by administration of this genetically modified antibody in combination with a dendritic cell maturation factor. Alternatively, the genetically modified antibody to DEC-205 may contain both the antigen sequence as well as the sequence of the dendritic cell maturation factor on the same polypeptide chain, either the light chain or the heavy chain of the antibody. This would allow for concurrent delivery of the antigen to the dendritic cell as well as maturation of the dendritic cell to allow for more efficient antigen presentation and subsequent robust and long lasting immune responses. As noted above, the methods may be carried out ex vivo, that is, the dendritic cells of the patient may be removed and exposed to the anti-DEC-205/antigen complex followed by maturation of the dendritic cells with the dendritic cell maturation factor outside of the body, followed by transfer of the mature dendritic cells back to the patient. Alternatively, the methods may be done in vivo by administration of the anti-DEC-205 antibody/antigen complex together with the dendritic cell maturation factor.

Accordingly, a first aspect of the invention provides a method of promoting highly efficient antigen presentation in a mammal comprising:

a) exposing ex vivo or in vivo dendritic cells from said mammal to either of the following:

i) a conjugate comprising a preselected antigen covalently bound to an antibody to DEC-205; or

ii) a recombinant anti-DEC-205 antibody, wherein said antibody has been genetically modified to contain at least one preselected antigen on at least one preselected site on said antibody molecule; and

b) promoting maturation of said dendritic cells ex vivo or in vivo by combining the antigen/anti-DEC-205 complex of either of i) or ii) of step a) with a dendritic cell maturation factor;

wherein the combination of steps a) and b) results in highly efficient antigen presentation in said mammal.

A second aspect of the invention provides a method of promoting highly efficient antigen presentation in a mammal comprising administering a recombinant anti-DEC-205 antibody to said mammal, wherein said antibody has been genetically modified to contain at least one preselected antigen and at least one dendritic cell maturation factor, each on at least one preselected site on said antibody, and wherein said administering results in delivery of said antigen to said dendritic cell, maturation of said dendritic cell and promotion of highly efficient antigen presentation.

In a particular embodiment, the method of promoting highly efficient and persistent antigen presentation in a mammal results in a robust and long lasting immune response, which may be cellular (T cell) or humoral (B cell) in nature. The T cells may be cytolytic T cells, helper T cells or memory T cells. In another embodiment, the methods provided herein result in generation of mucosal immunity. In yet another embodiment, the methods result in the predetermined antigen being about 500 times more effective in inducing a robust and long-lasting T cell response and in expanding antigen-specific CD4+ and CD8+T cells in the mammal, as compared to an antigen administered without conjugation to anti-DEC-205 antibody fragments and delivered without combining the antigen with a dendritic cell maturation factor prior to injection. Furthermore, the methods of the present invention increase the efficiency with which the predetermined antigen initiates CD4⁺ and CD8⁺ immunity from the polyclonal naive T cell repertoire in vivo. In yet another embodiment, the anti-DEC-205 antibody may be a polyclonal or a monoclonal antibody. In a preferred embodiment, the antibody is selected from the group consisting of a human antibody, a murine antibody (preferably one that reacts with human DEC-205 protein), a humanized antibody and a human chimerized antibody. In yet a further preferred embodiment, the methods are carried out with monovalent fragments of the antibodies, or single chain antibodies. In yet another particular embodiment, the preselected site on the antibody is on the heavy or light chain of the antibody, or on fragments thereof.

A third aspect of the invention provides a method of priming CD8+T cells with a non- replicating and/or subunit vaccine comprising:

a) exposing ex vivo or in vivo dendritic cells from a mammal to either of the following:

i) a conjugate comprising a non-replicating and/or subunit vaccine covalently bound to an antibody to DEC-205; or

ii) a recombinant anti-DEC-205 antibody, wherein said antibody has been genetically modified to contain at least one non-replicating and/or subunit vaccine on at least one preselected site on said antibody molecule; and

b) promoting maturation of said dendritic cells ex vivo or in vivo by combining the non- replicating and/or subunit vaccine/anti-DEC-205 complex of either of i) or ii) of step a) with a dendritic cell maturation factor;

wherein the combination of steps a) and b) results in highly efficient antigen presentation in said mammal and subsequent priming of CD8⁺ T cells.

In a particular embodiment, the vaccine is selected from the group consisting of a tumor vaccine, a viral vaccine, a bacterial vaccine and vaccines for other pathogenic organisms for which a vigorous and long lasting T cell response is necessary to provide long term protection from infection or disease. In another embodiment, the viral vaccine is a DNA viral vaccine, an RNA viral vaccine, a retroviral vaccine or a tumor vaccine formed with the antibody combining function of the anti-DEC-205 antibody. The vaccine may also be effective when administered without adjuvant. In a particular embodiment, the tumor vaccine, upon administration to an individual bearing said tumor, may result in tumor regression. In another particular embodiment, the tumor regression is a result of a robust and long-lasting T cell response specific for said tumor. In a further preferred embodiment, the T cell response is a cytolytic T cell response, a helper T cell response or a memory T cell response. In yet another embodiment, the viral, bacterial, or tumor vaccine is administered as a single dose sufficient to elicit a vigorous and long lasting T cell response. In a preferred embodiment, the single dose of vaccine is administered at levels of about 10 to 1000 fold lower than the level of a vaccine administered without an anti- DEC 205 antibody and without a dendritic cell maturation factor but with an adjuvant, results in highly efficient antigen presentation and induction of long lasting immune responses. In another embodiment, the vaccine is administered at a single dose of about 1 mg to about 10 mg. In yet another embodiment, the vaccine is administered at a single dose of about 1 μg to about 10 μg. In yet another embodiment, the vaccine is administered at a single dose of about 10 ng to about 100 ng. One embodiment provides for the vaccine to be administered subcutaneously, intramuscularly, intravenously, intranasally, orally, mucosally, bucally or sublingually.

A fourth aspect of the invention provides a method for increasing the persistence of MHC class I: antigen complexes in vivo comprising:

a) exposing ex vivo or in vivo dendritic cells from said mammal to either of the following:

i) a conjugate comprising a preselected antigen covalently bound to an antibody to DEC-205; or

ii) a recombinant anti-DEC-205 antibody, wherein said antibody has been genetically modified to contain at least one preselected antigen on at least one preselected site on said antibody molecule; and

b) promoting maturation of said dendritic cells ex vivo or in vivo by combining the antigen/anti-DEC-205 complex of either of i) or ii) of step a) with a dendritic cell maturation factor;

wherein the combination of steps a) and b) results in persistent presentation of antigen in the context of MHC class I antigens such that persistence of MHC class I: antigen complexes in said mammal results in induction of a long lasting T cell response specific for said antigen; and wherein such persistent presentation of antigen is analogous to a systemic infection as evidenced by presentation of antigen in most lymphoid tissue. In a preferred embodiment, the MHC class I: antigen complexes persist in vivo in multiple lymphoid sites from about 15 to about 30 days. In another preferred embodiment, such antigen presentation results in induction of mucosal immunity.

In a preferred embodiment, the antigen and anti-DEC-205 antibody mixture, combined with the dendritic cell maturation factor, is prepared in a composition formulated for delivery to a mucosal site for enhanced induction of antigen specific T and B cell responses. Such formulation may be delivered orally, intranasally, bucally or sublingually.

In a particular embodiment, the methods described above are associated with an increase in antigen specific CD8⁺ cytolytic T cell responses.

A fifth aspect of the invention provides for vaccine compositions for inducing long term cellular or humoral immunity in a mammal. In a particular embodiment, the mammal is selected from human and non-human mammals. In a preferred embodiment, the mammal to be treated is preferably a human, although use of the vaccine compositions in other mammals is also conceived.

In a particular embodiment, the vaccine composition comprises a mixture of:

a) an immunogenically effective amount of an antigen for which induction of long term cellular or humoral immunity is desired, conjugated to monovalent fragments of an anti-DEC-205 antibody;

b) a dendritic cell maturation factor;

c) a pharmaceutically acceptable adjuvant; and

wherein said vaccine composition is effective when administered at levels of about 10 to 1000 fold lower than the effective dose of a vaccine which is not conjugated to an anti-DEC-205 antibody or fragments thereof and which is not administered with a dendritic cell maturation factor.

In another embodiment, the vaccine composition is administered at a single dose of about 1 mg to about 10 mg. In yet another embodiment, the vaccine is administered at a single dose of about 1 μg to about 10 μg. In yet another embodiment, the vaccine is administered at a single dose of about 10 ng to about 100 ng. One embodiment provides for the vaccine to be administered subcutaneously, intramuscularly, intravenously, intranasally, orally, mucosally, bucally or sublingually.

In yet another embodiment, the vaccine composition is a DNA vaccine composition comprising:

a) an isolated DNA molecule comprising at least one nucleotide sequence encoding at least one antigenic polypeptide isolated from a virus, bacterium or tumor cell against which immunity is desired;

b) an isolated DNA molecule comprising at least one nucleotide sequence encoding an ant i-DEC-205 antibody or a DEC-205 binding fragment thereof;

c) a pharmaceutically acceptable carrier; and

wherein said composition, when administered with a dendritic cell maturation factor at levels of about 10 to 1000 fold lower than the effective dose of an antigenic polypeptide which is not conjugated to an anti-DEC-205 antibody or fragments thereof and which is not administered with a dendritic cell maturation factor, results in efficient, vigorous and long lasting cellular and humoral immunity specific for said virus, bacterium or tumor cell. In a particular embodiment, the nucleotide sequence encoding an anti-DEC-205 antibody or fragment thereof is selected from the nucleotide sequences set forth in SEQ ID NOS: 13 and 14, wherein said nucleotide sequences encode the heavy or light chain variable region of an anti-DEC-205 antibody.

A sixth aspect of the invention provides an immunogenic composition which, upon administration to a mammal with or without the use of an adjuvant at doses 10 to 1000 fold lower than the doses normally administered to mammals with known adjuvants, provides for robust and long lasting cellular or humoral immunity in the mammal. In a preferred embodiment, the immunogenic composition may be used to vaccinate individuals against specific pathogens for which immunity is desired. In a preferred embodiment, the immunogenic composition may be delivered at least once at levels sufficient to induce a long lasting T cell response. In another preferred embodiment, the T cell response may be a cytolytic T cell response, a helper T cell response or a memory T cell response.

In another preferred embodiment, the composition comprises:

a) an immunogenically effective amount of an antigen for which induction of non-mucosal or mucosal T cell or B cell immunity is desired, conjugated to monovalent fragments of an anti-DEC-205 antibody;

b) a dendritic cell maturation factor;

c) a pharmaceutically acceptable adjuvant;

d) a means for delivering said composition; and

wherein said composition results in generation of antigen specific antibodies and/or CD8+ cytolytic T cells, when administered at levels of about 10 to 1000 fold lower than the effective dose of a composition wherein the antigen is not conjugated to an anti-DEC-205 antibody or fragments thereof and which is not administered with a dendritic cell maturation factor and wherein said T cell or B cell responses are vigorous and long-lasting.

In another particular embodiment, the immunogenic composition is a recombinant immunogenic composition comprising a nucleic acid molecule comprising:

a) a first nucleotide sequence encoding a chain of an antibody specific for DEC-205;

b) a second nucleotide sequence encoding at least one antigen from a virus, a bacterium, or a tumor cell against which immunity is desired;

c) a third nucleotide sequence encoding a dendritic cell maturation factor;

d) a fourth nucleotide sequence comprising a promoter for expression of a fusion protein comprising said anti-DEC-205 antibody, said antigen and said dendritic cell maturation factor; and

e) a pharmaceutically acceptable carrier.

In a preferred embodiment, the antibody of the compositions may be a polyclonal antibody, a monoclonal antibody, a chimeric or hybrid antibody, a human chimerized antibody or monovalent fragments thereof. In another preferred embodiment, the antibody chain is the light chain or heavy chain or fragments thereof. The antibody may be selected from the group consisting of a human or humanized antibody, a mouse antibody, a rat antibody, a horse antibody, a goat antibody, a sheep antibody, and monovalent fragments thereof. The antibody may be a single chain antibody. In the recombinant composition, transcription of the first, second and third nucleotide sequences may be under the control of one promoter. Alternatively, in the recombinant composition, transcription of the first, second and third nucleotide sequences may be under the control of individual promoters.

A seventh aspect of the invention provides a method for immunizing a mammal, comprising administering to said mammal a composition comprising:

a) an immunogenically effective amount of a sub-unit vaccine comprising a combination of an isolated polypeptide obtained from a pathogen or a tumor cell against which immunity is desired, conjugated to monovalent fragments of an anti-DEC-205 antibody;

b) a dendritic cell maturation factor;

c) a pharmaceutically acceptable carrier, and

wherein said sub-unit vaccine, when administered with a dendritic cell maturation factor at levels of about 10 to 1000 fold lower than the effective dose of a sub-unit vaccine which is not conjugated to an anti-DEC-205 antibody or fragments thereof and which is not administered with a dendritic cell maturation factor, results in efficient, vigorous and long lasting cellular and humoral immunity specific for said sub-unit vaccine.

In a particular embodiment, the polypeptide may be derived from a bacteria, a virus, a tumor cell, or any other pathogen for which immunity is desired.

An eighth aspect of the invention provides a method for long term protection of a mammal from infection with a pathogen or a tumor cell.

In a particular embodiment, the method for long term protection of a mammal comprises administering an immunogenically effective amount of a vaccine comprising:

a) a vector containing a gene encoding a protein or polypeptide from a pathogen or tumor cell or an immunogenic fragment thereof, operatively associated with a promoter capable of directing expression of the gene in the mammal; and

b) a vector containing a gene encoding the light or heavy chain anti-DEC-205 antibody operatively associated with a promoter capable of directing expression of the gene in the mammal;

c) a vector containing a gene encoding a dendritic cell maturation factor, operatively associated with a promoter capable of directing expression of the gene in the mammal; and

d) a pharmaceutically acceptable adjuvant.

In another embodiment, the method for long term protection of a mammal from infection with a pathogen or a tumor cell comprises administering an immunogenically effective amount of a vaccine comprising:

a) a vector containing a gene encoding a protein or polypeptide from a pathogen or tumor cell or an immunogenic fragment thereof, operatively associated with a promoter capable of directing expression of the gene in the mammal;

b) a vector containing a gene encoding the light or heavy chain of an anti-DEC-205 antibody operatively associated with a promoter capable of directing expression of the gene in the mammal;

c) a pharmaceutically acceptable adjuvant; and

wherein said method further comprises administering the components of steps a), b) and c) with a dendritic cell maturation factor, wherein said administering results in long term protection of a mammal from infection with a pathogen or tumor cell.

A ninth aspect of the invention provides a virus-like particle (VLP) comprising:

a) at least one immunogenic polypeptide from a virus against which immunity is desired conjugated to monovalent fragments of an anti-DEC-205 antibody;

b) a dendritic cell maturation factor;

c) a pharmaceutically acceptable adjuvant; and

wherein said virus like particle, when administered at an immunogenically effective amount with a dendritic cell maturation factor at levels of about 10 to 1000 fold lower than the effective dose of a virus-like particle which contains at least one immunogenic polypeptide from a virus against which immunity is desired and which is not conjugated to an anti-DEC-205 antibody or fragments thereof and which is not administered with a dendritic cell maturation factor, results in efficient, vigorous and long lasting cellular and humoral immunity specific for said virus.

In a particular embodiment, the VLP contains at least one immunogenic polypeptide which may be obtained from a virus selected from the group consisting of a DNA virus, an RNA virus and a retrovirus. In another embodiment, the VLP may be used for immunizing an animal against a virus, wherein the administering of the VLP results in induction of long term T cell, B cell or mucosal immunity, and subsequent protection of the mammal from infection with the virus. In addition, another embodiment provides for use of the VLP for treating certain tumors that result from infection with certain oncogenic viruses. As such, the VLP can be used as a tumor cell vaccine.

In the methods described above, the preferred dendritic cell maturation factor may be selected from the group consisting of an anti-CD40 antibody, an inflammatory cytokine, poly I/C, single strand RNA, DNA, CpG, ligation of the IL-1, TNF or TOLL-like receptor families, and activation of an intracellular pathway leading to dendritic cell maturation such as TRAF-6 or NF-KB.

Furthermore, the methods described above provide for a conjugate of the antigen and antibody specific for the cell surface protein on the antigen presenting cell, such as DEC-205, wherein such conjugate may be prepared using standard chemical means. Alternatively, the antigen and antibody may be expressed together on one polypeptide chain for administration to the mammal, accompanied by administration of the dendritic cell maturation factor, such that the antibody serves to direct the antigen to the antigen presenting cell and administration of the maturation factor allows efficient antigen presentation as well as induction of a highly efficient, robust and long lasting immune response. In another particular embodiment, the antigenic polypeptide, the anti-DEC-205 antibody or monovalent fragment thereof, and the dendritic cell maturation factor polypeptide may be on one polypeptide chain, so that delivery of the antigen to the dendritic cell via the antibody also results in concurrent maturation of the dendritic cell. Such an approach can be taken using standard molecular techniques known to one skilled in the art.

Various routes of delivery are embraced herein, including but not limited to enteral or parenteral delivery. Transmucosal delivery, e.g., orally, intranasally, bucally, sublingually or rectally is also contemplated as is transdermal delivery. Parenteral includes but is not limited to, subcutaneous, intravenous, intra-arterial, intramuscular, intradermal, intraperitoneal, intraventricular, and intracranial administration. Pulmonary, intraintestinal, and delivery across the blood brain barrier are also embraced herein.

Administration as a vaccine for enhancement of an immune response is a preferred embodiment.

A tenth aspect of the invention provides a recombinant anti-DEC-205 molecule, comprising an antibody reactive with DEC-205 which has been genetically modified to contain at least one preselected antigen on at least one site on said antibody molecule, and at least one dendritic cell maturation factor on at least one site on said antibody molecule, wherein said antibody molecule, upon administration to a mammal, is capable of delivering said antigen to antigen presenting cells expressing DEC-205 and wherein said delivery results in highly efficient antigen presentation and induction of long term cellular and/or humoral immunity. In a preferred embodiment, the delivery of the antigen via this recombinant molecule, results in a robust and long lasting T cell response, which may be selected from the group consisting of a cytolytic T cell response, a helper T cell response and a memory T cell response. In a preferred embodiment, the at least one site may be on either the heavy chain or the light chain of the anti-DEC-205 antibody. In another preferred embodiment, the recombinant anti-DEC-205 molecule comprises amino acid sequences consisting of human anti-DEC-205 antibody sequences or murine anti-DEC-205 antibody sequences which react with human DEC-205 protein.

An eleventh aspect of the invention provides a method for enhancing the development of tolerance to a preselected antigen by delivering the preselected antigen to a DEC-205 receptor on an antigen-presenting cell having a DEC-205 receptor in the absence of DC maturation. Methods and conjugates for delivering the antigen are as described above. Non-limiting examples of antigens for which tolerance of the immune system is desirable include transplant antigens, allergens, and antigens toward which autoimmunity has or may develop. In one embodiment, the use of ligands that are recognized by the C-type lectin and other domains of the DEC-205 receptor, including such modifications of vaccines that are recognized by DEC-205 receptor, such as modified tumor cells and tumor antigens, microbial vectors and associated antigens, and autoantigens.

In a particular embodiment, the methods for inducing tolerance to a preselected antigen may comprise exposure of dendritic cells ex vivo or in vivo to an antigen against which tolerance is desired, the antigen of which has been either conjugated chemically to an anti-DEC-205 antibody, or has been recombinantly expressed on the same polypeptide chain as the anti-DEC-205 antibody. Unlike the methods for induction of an immune response to a particular antigen, the methods for tolerance induction to an antigen do not include the need for any maturation factors for the dendritic cells. Thus, the methods for tolerance induction omit this particular step or inclusion of any such factors, such as CD40 ligation etc. as noted above.

Delivering the preselected antigen to the endocytic receptor is carried out by exposing the antigen presenting cell to a conjugate or complex between a molecule that binds the endocytic receptor, and the antigen. In the instance where the endocytic receptor is DEC-205, the method is carried out by exposing the antigen-presenting cell to a conjugate that includes both a DEC-205-binding molecule and a preselected antigen. Alternatively, the antigen may be expressed by recombinant means on the same polypeptide chain as the antibody, which is prepared by using a vector containing a gene encoding a protein or polypeptide from a pathogen or tumor cell or an immunogenic fragment thereof, operatively associated with a promoter capable of directing expression of the gene in the mammal; and a vector containing a gene encoding the light or heavy chain of an anti-DEC-205 antibody operatively associated with a promoter capable of directing expression of the gene in the mammal; and a pharmaceutically acceptable adjuvant; and administering these components with a dendritic cell maturation factor in a pharmaceutically acceptable carrier or adjuvant. In certain embodiments, the use of an adjuvant is optional. Alternatively, rather than administering the dendritic cell maturation factor separately, the recombinant vaccine composition may also contain a third vector containing a gene encoding a dendritic cell maturation factor, operatively associated with a promoter capable of directing expression of the gene in the mammal. The expression of the antigen, the antibody and the dendritic cell maturation factor may be under the control of individual promoters or under the control of one promoter. Thus, upon delivery to a subject in which immunity to a specific pathogen is desired, the recombinant vaccine will encompass the antigen, the antibody for enhancing delivery to a dendritic cell having a specific receptor for DEC-205 on its surface, as well as the dendritic cell maturation factor necessary for increasing maturation of the cell and for enhanced antigen presentation and subsequent induction of highly efficient and long lasting immune responses, particular T cell responses. It is to be noted, however, that for those antigens for which B cell immunity is the primary protection desired, the methods of the present invention will also be highly beneficial, since the enhanced helper T cell responses noted to occur using the techniques described herein, will also be beneficial in the desired enhancement of B cell responses. As will be seen below, the antigen may be any compound, molecule, or substance desirably enhancedly delivered to an antigen-presenting cell, such as a protein, peptide, carbohydrate, polysaccharide, lipid, nucleic acid, cell, by way of non-limiting examples. Various means of conjugating or complexing the antigen to the endocytic receptor-binding molecule is embraced herein, including but not limited to covalent cross-linking, and in the instance where both molecules are proteins or peptides, expression together in a single-chain polypeptide using recombinant methods known to one skilled in the art.

In the instance where the endocytic receptor is DEC-205, the DEC-205-binding molecule may be any ligand for DEC-205, including antibodies or natural ligands. In a preferred embodiment, the DEC-205-binding agent is an antibody, and most preferably a monoclonal antibody, such as, but not limited to, NLDC-145. In another preferred embodiment, the antibody is a murine, rabbit or human polyclonal antibody that reacts with human DEC-205 protein or a monoclonal antibody other than NLDC-145 that binds to or reacts with human DEC-205 protein. However, natural ligands to DEC-205 may be utilized, examples of which are described herein, wherein conjugation or covalently coupling the preselected antigen thereto is also embraced by the present invention.

The antigen may be any compound, substance or agent for which a modulated immune response is desired or for which enhanced delivery into antigen-presenting cells is desired. Such antigens may include proteins, cells, nucleic acids including DNA, RNA, and antisense oligonucleotides, carbohydrates, polysaccharides, lipids, glycolipids, among others. Non-limiting examples include immunogenic portions of DNA or RNA viruses, or of retroviruses. Particular non-limiting examples include HIV-1, HPV, EBV, HSV, influenza virus and SARS virus. Also contemplated are immunogenic portions of Mycobacterium tuberculosis and malaria, for use in a vaccine to enhance the development of an immune response thereto. In the instance where tolerization to an antigen is desired in order to prevent a potential immune response, such antigens include transplant antigens, allergens and autoimmune antigens, by way of non-limiting example.

To enhance the development of an immune response to the antigen delivered via the DEC-205 receptor, DC maturation or exposure of the DC to a maturation signal may be achieved in any of a number of ways. In the example in which CD40 ligation is used, it may be achieved by exposing the antigen-presenting cell ex vivo or in vivo to an agonistic anti-CD40 antibody, although other methods and agents for achieving CD40 ligation are embraced herein. Exposure of DCs to other maturation signals in the form of agonistic antibodies to other receptors is embraced herein. Activation of intracellular DC maturation signals may be achieved by, for example, by ligands that signal Toll like receptors, e.g., CpG oligodeoxynucleotides, RNA, bacterial lipoglycans and polysaccharides, TNF receptors such as the TNFα receptor, IL-1 receptors, and compounds that activate an intracellular pathway leading to dendritic cell maturation, such as TRAF 6 or NF-κB signaling pathways. Both natural ligands for DEC-205 as well as antibodies may be used.

The present invention is also directed to conjugates between an antigen-presenting cell endocytic receptor-binding molecule and a preselected antigen for the aforementioned purposes, and pharmaceutical compositions comprising such conjugates. Non-limiting examples of the antigen-presenting cell is a dendritic cell, and of endocytic receptors, DEC-205, the asialoglycoprotein receptor, the Fcγ receptor, the macrophage mannose receptor, and Langerin. As noted above, the conjugates may be a covalently cross-linked or a conjugate between the receptor-binding molecule and a preselected antigen. Alternatively, the amino acid sequence for the antigen may be recombinantly expressed on either the light or the heavy chain of the anti-DEC-205 antibody and exposed to dendritic cells either in vivo or ex vivo along with a dendritic cell maturation factor. The antigen may be any material, substance or compound for which enhanced delivery to an antigen-presenting cell, such as dendritic cell is desired, including but not limited to proteins, cells, nucleic acids such as DNA and RNA, carbohydrates, etc. In the embodiment wherein the preselected antigen is a peptide antigen or a protein antigen, and the endocytic receptor-binding molecule is a protein, such as an antibody or protein ligand, the antigen and the binding protein may reside on'the same polypeptide chain. In a preferred embodiment, the endocytic receptor is DEC-205, and the DEC-205-binding protein is an antibody, preferably one that reacts with human DEC-205. In another embodiment, the antigen is recognized directly by the DEC-205 multilectin receptor.

The invention is also directed to polynucleotides encoding the aforementioned single-chain chimeric polypeptides.

As noted above, the enhanced delivery of molecules to an antigen-presenting cell such as a dendritic cell is achieved by coupling the molecule to, for example, a DEC-205-targeting agent. In addition to enhanced antigen delivery, targeting of nucleic acids to antigen-presenting cells via an endocytic receptor such as DEC-205 is a means for introducing foreign DNA into an antigen-presenting cell for transfection or other gene therapy purposes. It need not be associated with DC maturation or absence of DC maturation thereof to achieve this embodiment of the invention.

Other antigen-presenting cell endocytosis receptors other than DEC-205 are likewise targets for enhanced antigen-presenting cell delivery, such as but not limited to the asialoglycoprotein receptor, the Fcγ receptor, the macrophage mannose receptor, and Langerin. All of the aforementioned uses of DEC-205, and compositions comprising a DEC-205-targeted molecule and an antigen respectively pertain to other endocytosis receptors.

It is thus an object of the invention to provide a method for enhancing the development of a long lasting cellular immune response to a preselected antigen comprising delivering the preselected antigen to an endocytic receptor on a dendritic cell and inducing promoting maturation of the dendritic cell. In one embodiment, the endocytic receptor is DEC-205. The delivering of the preselected antigen to DEC-205 may include at least exposing the dendritic cell to a DEC-205-binding agent comprising the preselected antigen. The DEC-205-binding agent including at least the preselected antigen may be a conjugate between said DEC-205-binding agent and said preselected antigen. In a preferred embodiment, the preselected antigen may be a peptide antigen or a protein antigen, and the peptide or protein antigen may be conjugated to the DEC-205-binding agent by means of a cross-linking agent. In the instance where the DEC-205-binding agent is a protein, it is a further object of the invention to provide a DEC-205-binding agent and a peptide antigen or protein antigen on a single polypeptide chain. In a preferred embodiment, the DEC-205-binding agent may be an antibody.

It is a further object of the invention to enhance the development of an immune response to the antigen by inducing maturation of the dendritic cell with CD40 ligation. CD40 ligation may be achieved by exposing the dendritic cell to an agonistic anti-CD40 antibody. The delivering of the preselected antigen to DEC-205 and promoting dendritic cell maturation in the dendritic cell may be independently carried out ex vivo or in vivo.

It is yet a further object of the invention to provide a method for enhancing the development of tolerance to a preselected antigen by at least delivering the preselected antigen to an endocytic receptor on a dendritic cell in the absence of dendritic cell maturation. The endocytic receptor may be DEC-205. The delivering of the preselected antigen to the DEC-205 may be carried out by at least exposing the dendritic cell to a DEC-205-binding agent that contains the preselected antigen. The DEC-205-binding agent that contains the preselected antigen may be a conjugate between the DEC-205-binding agent and the preselected antigen. In the case in which the preselected antigen is a peptide antigen or a protein antigen, the conjugate of the DEC-205-binding agent may be by means of a cross-linking agent. Where the DEC-205-binding agent is a protein, the DEC-205-binding agent and the peptide antigen or protein antigen may be present on a single polypeptide chain. In a preferred embodiment, the DEC-205-binding agent may be an antibody. In the foregoing method, agents that block intracellular signalling at the levels of TRAF 6 and NF-κB, which are used by CD40 and Toll-like receptors and IL-1r to trigger dendritic cell maturation.

It is still yet a further object of the invention to provide a conjugate for enhanced delivery of a preselected antigen to a dendritic cell, the conjugate being at least a covalent complex between a binding molecule to an endocytic receptor and the antigen. The endocytic receptor may be DEC-205. The binding molecule to DEC-205 may be an antibody to DEC-205. In one embodiment, the antigen may be covalently bound to the antibody to DEC-205 via a cross-linking agent. The antigen may be a peptide or a protein. In another embodiment, the peptide or protein and a light chain or a heavy chain of the antibody to DEC-205 may reside on the same polypeptide chain, forming a chimeric polypeptide. This chimeric polypeptide molecule may be produced using standard recombinant molecular biology techniques known to one skilled in the art and comprises a genetically modified antibody molecule, an antigen and optionally, a dendritic cell maturation factor when immunity to the antigen is desired. The dendritic cell maturation factor is not necessary when tolerance to the antigen is desired. Alternatively, the chimeric polypeptide may comprise the anti-DEC-205 antibody and the antigen sequence and this complex may then be exposed to the dendritic cell in vivo or ex vivo, and may include concurrent administration of a dendritic cell maturation factor.

It is another object of the invention to provide polynucleotides that encode the chimeric polypeptides mentioned above. Such polynucleotides may comprise the nucleic acid sequences for the anti-DEC-205 antibody or fragments thereof, the nucleic acid sequences that encode the antigen for which an immune response or tolerance is desired, and in the case where dendritic cell maturation is desired (that is, for when an immune response is desired), the nucleic acid that encodes the maturation factor. In one embodiment, it is envisioned that separate promoters may be used for each component of the chimeric polypeptide, that is, for the antigen, the antibody and the maturation factor. Alternatively, a single promoter may be used for all three nucleic acid moieties.

It is yet still an even further object of the invention to provide a method for enhancing the delivery of a preselected antigen to a dendritic cell by at least exposing the dendritic cell to the conjugate or chimeric polypeptide described above. Non-limiting examples of the foregoing antigens include a protein, cell, nucleic acid, carbohydrate, polysaccharide, lipid, or glycolipid. The nucleic acid may be DNA, RNA or an antisense oligonucleotide.

These and other aspects of the present invention will be better appreciated by reference to the following drawings and Detailed Description.

### BRIEF DESCRIPTION OF THE DRAWINGS

FIGS. 1A-E show that the monoclonal antibody NLDC-145 targets DCs in vivo.

FIGS. 2A-B show that DCs process and present antigen delivered by hybrid antibodies comprising amino acids 46-61 of hen white lysozyme added to the carboxy terminus of cloned NLDC145 monoclonal antibody to DEC-205 (αDEC/HEL).

FIGS. 3A-E demonstrate in-vivo activation of CD4⁺ T cells by αDEC/HEL.

FIGS. 4A-C shows that CD4⁺ T cells divide in response to antigen presented by DCs in vivo, produce IL-2 but not IFN-γ and are then rapidly deleted.

FIGS. 5A-C show that CD40 ligation prolongs T cell activation in response to antigens delivered to DCs and induces up-regulation of co-stimulatory molecules on DCs.

FIG. 6 Characterization of monoclonal IgG:OVA conjugates. (A) IgG:OVA conjugates at various stages of conjugation. Nonreduced gel (left) of the 80 kDa monovalent IgG after MESNA treatment, and reduced and boiled (right) to show heavy and light chains. (B) Western analysis of antibody (DEC-205 and III/10 isotype control) OVA conjugates. (C)C57BL/6 or DEC-205^{-/-} mice were injected i.v. with 106 CFSE-labeled OT-I or OT-II T-cells and 24 hrs later with either antibody conjugates containing 50 ng of OVA or 25 μg soluble OVA s.c. 3 days later, proliferation in lymph nodes was evaluated by flow cytometry. (D) As in C, but graded doses of OVA conjugated to IgG or endotoxin free OVA were used. Representative of 2 or more experiments.

FIG. 7 αDEC-205:OVA with αCD40 primes both CD4⁺ and CD8⁺ T cells in vivo. (A) αDEC-205:OVA containing 500 ng of OVA was administered to näive C57BL/6 mice s.c. with 25 μg of αCD40. 7 days later, spleen cell suspensions were CFSE labeled and restimulated in vitro for 5 days with LPS-free OVA (500 μg/mL) to evaluate proliferation by flow cytometry. (B) As in (A), but the cells were restimulated with either SIINFEKL (SEQ ID NO: 15) (1.0 μM) or LSQAVHAARAEINEAGR (SEQ ID NO: 16) (2.0 μM) peptides for 2 days and IFN-γ secretion evaluated by ELISPOT. (C) Mice were immunized with grade doses of OVA as a soluble protein or conjugated to αDEC-205. IFNγ secretion was evaluated after 7 days in the lymph nodes and spleen as in (B). Representative of at least 2 experiments.

FIG. 8 αDEC-205:OVA in combination with αCD40 induces durable and strong OVA-specific responses by CD8⁺ T cells. (A) αDEC-205:OVA containing 50 ng of OVA was administered to naïve C57BU6 mice s.c. with 25 μg of αCD40, 14, 21, 60 and 90 days later, intracellular IFN-γ staining was evaluated by flow cytometry without or with OVA peptide restimulation. Indicated percentages are percent IFN-γ⁺CD8⁺ cells. (B) Wild type, DEC-205^{-/-}, CD8^{-/-} and CD4^{-/-}mice were treated as in A. 14 days later, 7×10⁶ of each, CFSE-labeled syngeneic splenocytes pulsed with peptide (CFSE^{hu}) or not (CFSE^{lo}), were injected i.v. to detect active killer cells in the lymph nodes. (C) As in (B), but mice were evaluated after 60 days. Data are representative of 2 or more experiments.

FIG. 9 Enhanced efficacy of αDEC-205:OVA plus αCD40 relative to other immunization approaches. (A) C57BL/6 mice were immunized s.c. with several methods: spleen DC pulsed ex vivo with 10 μg/mL each of αDEC-205:OVA and αCD40; 500 μg OVA in CFA; 50 μg OVA with 25 μg αCD40; 50 μg of SIINFEKL peptide with 25 μg αCD40; or 50 ng of OVA in αDEC-205:OVA with 25 μg of αCD40. 7 or 30 days later, lymph nodes were harvested and T cell expansion evaluated by K^{b}-SIINFEKL:PE tetramer and CD62L staining. The gate for the y-axis was placed relative to the CD62L negative tetramer binding cells in the right panel. Indicated percentages are percent of CD8⁺ lymphocytes. (B) As in A, but IFN-γ secretion evaluated by intracellular cytokine staining. Data are means of 3 experiments.

FIG. 10 Systemic antigen presentation following DEC-205 targeting in situ. (A) C57BL/6 mice were given 10 μg of Alexa₄₈₈ conjugated antibodies s.c. At the indicated time points, CD11c⁺ cells were enriched from the draining or distal lymph nodes or spleen for evaluation by flow cytometry. The frequencies of DCs capturing the injected Ig's are shown, and the DEC-205 and CD8 high subset of splenic DCs arrowed. (B) C57BL/6 mice were given 10 μg of αDEC-205:OVA, isotype:OVA or PBS s.c. and, after 18 hrs, CD11c⁺ cells were enriched from draining or distal lymph nodes or spleen. The presence of OVA was evaluated by intracellular staining with Alexa₄₈₈ conjugated αOVA and flow cytometry. (C) 15 hrs after s.c. treatment with 5 μg of αDEC-205:OVA or the isotype conjugate±αCD40, CD11c⁺ lymph node or spleen DCs were selected and used to stimulate OT-I T cells without further addition of OVA. (D) As in (C), but mice were treated with αCD40 and either αDEC-205:OVA (5 μg), OVA (500 μg) or PBS. Data are representative of at least 2 experiments.

FIG. 11 Prolonged MHC-I but not MHC-II presentation following DEC-205 targeting in situ. (A) C57BL/6 mice were immunized to OVA under the conditions listed above each panel for 15, 7, 3, or 1 day prior to transferring 10⁶ CFSE-labeled OT-I T cells. Proliferation in the lymph nodes was monitored by flow cytometry 3 days later. (B) As in (A), but CFSE labeled OT-I or OT-II T cells were transferred. (C)C57BL/6 mice were treated with 50 ug MHC I binding peptide (SIINFEKL (SEQ ID NO: 15) in CFA, 50 ug MHC II binding peptide (LSQAVHAAHAEINEAGR (SEQ ID NO: 16)) in CFA, CFA alone, or PBS. IFN-γ secretion was evaluated after 12 days in the lymph nodes as in 2B. Data are representative of at least 2 experiments.

FIG. 12 Immunization with a single low dose of αDEC-205:OVA and αCD40 elicits resistance to OVA-modified pathogens. (A) C57BL/6 mice were vaccinated as described in 8A. 60 days later, mice were challenged with 5×10⁶ MO4 cells s.c. and tumor growth evaluated. (B) C57BL/6 mice were inoculated with MO4 tumor cells as in (A). 7 days later, mice were treated as in 4A and tumor growth evaluated. (C)C57BL/6 mice were treated as in 8A. 30 days after vaccination, mice were challenged with 10⁵ PFU of vaccinia-OVA intranasally. 7 days later, lungs were harvested and virus titer evaluated by a plaque-forming assay. (D) As in (C), but mice were weighed daily following viral challenge. Data are representative of at least 2 experiments.

FIG. 13 The DNA sequences of the V region of anti-human DEC-205 antibody lambda chain (SEQ ID NO: 13) and heavy chain (SEQ ID NO: 14).

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

Definitions

The terms used herein have the meanings recognized and known to those of skill in the art, however, for convenience and completeness, particular terms and their meanings are set forth below.

The term “antibody” as used herein includes intact molecules as well as fragments thereof, such as Fab and F(ab′)₂, which are capable of binding the epitopic determinant. Antibodies that bind the proteins of the present invention can be prepared using intact polypeptides or fragments containing small peptides of interest as the immunizing antigen attached to a carrier molecule. Commonly used carriers that are chemically coupled to peptides include bovine or chicken serum albumin, thyroglobulin, and other carriers known to those skilled in the art. The coupled peptide is then used to immunize the animal (e.g, a mouse, rat or rabbit). The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be a single chain antibody. (See, e.g., Curiel et al., U.S. Pat. No. 5,910,486 and U.S. Pat. No. 6,028,059). The various portions of the chimerized antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al, U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; and Queen et al., U.S. Pat. Nos. 5,585,089, 5,698,761 and 5,698,762. See also, Newman, R. et al., BioTechnology, 10: 1455-1460 (1992), regarding primatized antibody, and Ladner et al., U.S. Pat. No. 4,946,778 and Bird, R. E. et al., Science, 242: 423426 (1988)) regarding single chain antibodies. The antibody may be prepared in, but not limited to, mice, rats, rabbits, goats, sheep, swine, dogs, cats, or horses.

The term “antibody homologue” as used herein refers to whole immunoglobulin molecules, immunologically active portions or fragments thereof and recombinant forms of immunoglobulin molecules, or fragments thereof, that contain an antigen binding site which specifically binds (immunoreacts with) an antigen (e.g., cellular protein or protein from a pathogen or tumor). Additionally, the term antibody homologue is intended to encompass non-antibody molecules that mimic the antigen binding specificity of a particular antibody. Such agents are referred to herein as “antibody mimetic agents”.

Structurally, the simplest naturally occurring antibody (e.g., IgG) comprises four polypeptide chains, two heavy (H) chains and two light (L) chains inter-connected by disulfide bonds. It has been shown that the antigen-binding function of an antibody can be performed by fragments of a naturally-occurring antibody. Thus, these antigen-binding fragments are intended to be encompassed by the term “antibody homologue”. Examples of binding fragments include (i) a Fab fragment consisting of the VL, VH, CL and CH1 regions; (ii) a Fd fragment consisting of the VH and CH1 regions; (iii) a Fv fragment consisting of the VL and VH regions of a single arm of an antibody, (iv) a dAb fragment, which consists of a VH region; (v) an isolated complimentarity determining region (CDR); and (vi) a F(ab′)₂ fragment, a bivalent fragment comprising two Fab fragments linked by a disulfide bridge at the hinge region.

Furthermore, although the two regions of the Fv fragment are coded for by separate genes, a synthetic linker can be made that enables them to be made as a single chain protein (referred to herein as single chain antibody or a single chain Fv (scFv); see e.g., Bird et al. (1988) Science 242:423426; and Huston et al. (1988) Proc. Natl. Acad. Sci. USA 85:5879-5883). Such single chain antibodies are also encompassed within the term “antibody homologue”. Other forms of recombinant antibodies, such as chimeric, humanized and bispecific antibodies are also within the scope of the invention.

As used herein, the term “single-chain antibody” refers to a polypeptide comprising a V_{H} region and a V_{L} region in polypeptide linkage, generally linked via a spacer peptide (e.g., [Gly-Gly-Gly-Gly-Ser]ₓ), and which may comprise additional amino acid sequences at the amino-and/or carboxy-termini. For example, a single-chain antibody may comprise a tether segment for linking to the encoding polynucleotide. As an example, a scFv (single chain fragment variable) is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp.361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope).

The term “antibody combining site”, as used herein refers to that structural portion of an antibody molecule comprised of a heavy and light chain variable and hypervariable regions that specifically binds (immunoreacts with) antigen.

The terms “bind”, “immunoreact” or “reactive with” in its various forms is used herein to refer to an interaction between an antigenic determinant-containing molecule (i.e., antigen) and a molecule containing an antibody combining site, such as a whole antibody molecule or a portion thereof, or recombinant antibody molecule (i.e., antibody homologue).

The term “monoclonal antibody” or “monoclonal antibody composition”, as used herein, refers to a population of antibody molecules that contain only one species of an antigen binding site capable of immunoreacting with a particular epitope of an antigen. A monoclonal antibody composition thus typically displays a single binding affinity for a particular antigen with which it immunoreacts.

“Antibody fragments” recognizing DEC-205, as used herein, may be any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. The antibody fragment may be produced by any means. For instance, the antibody fragment may be enzymatically or chemically produced by fragmentation of an intact antibody or it may be recombinantly produced from a gene encoding the partial antibody sequence. As used herein, antibody also includes bispecific and chimeric antibodies. A single chain antibody molecule may be considered a monovalent fragment of an antibody.

The term “immunogen” is used herein to describe a composition typically containing a peptide or protein as an active ingredient (i.e., antigen) used for the preparation of antibodies against the peptide or protein.

The term “highly efficient”, as used in the present application, refers to the fact that an antigen, when combined with a ligand for DEC-205, for example, an antibody to the DEC-205 endocytic receptor, by either chemical means of conjugation, or by recombinant means, such that the antigen and antibody are expressed on the same polypeptide chain as a chimeric polypeptide, in addition to a dendritic cell maturation factor, is much more effective at antigen presentation and subsequent induction of immune responses (such as a highly proliferative T cell response) at much lower doses and in a much shorter time frame than those that are generally needed for antigen presentation and induction of immunity in the absence of the anti-DEC-205 antibody and dendritic cell maturation. For example, the inventors of the present application demonstrate that the antigens delivered with anti-DEC-205 antibody showed the ability to induce vigorous T cell proliferation, that is, reporter T cells labeled with CFSE prior to injection of the antigen/DEC-205 antibody conjugate, proliferated vigorously (5-7 division cycles) compared to an antigen delivered with isotype matched control antibody, which did not induce cell division. Furthermore, conjugated antigen was 1000 times more reactive at MHC class I presentation and 50 times more reactive at MHC class II presentation as shown by the ability of the conjugate to elicit a proliferative T cell response in vivo, whereas the non-conjugated antigen did not induce T cell proliferation (as shown using reporter T cells). Accordingly, significantly lower doses of antigen can be used to elicit antigen specific T cell proliferation. Furthermore, the term “highly efficient” also refers to the fact that once vigorous T cell proliferation is observed, the immune response is long lasting and may persist for as long as 3 months. Furthermore, the response may last this long even in the absence of a booster injection. The immune response may be a cellular (T cell) or humoral (B cell) immune response. Based on the data presented herein, “long lasting” or “long term” refers to the fact that about 75-80% of the immune reactivity of T cells may be measurable at 60 days post injection, and by 90 days post injection at least 30% T cell reactivity remains as measured by a cytolytic T cell assay or by T cell proliferation.

“Persistence of MHC class I: antigen complexes”, as used herein refers to the presence of antigen complexed with MHC molecules on dendritic cells for periods of greater than 3-5 days in lymph nodes, preferably for about 7-15 days. Furthermore, the persistence of MHC class I:antigen complexes result in the ability of very low doses of antigen (about 1000 fold lower than normally used for vaccine injection), being capable of induction of T cell proliferation even up to 15 days after injection.

The term “analogous to a systemic infection” refers to the fact that the methods of the present invention allow for antigen exposure for long periods of time to various cells of the immune system in lymphoid tissue throughout the body, thus mimicking what generally occurs during an active infection. The result of such extended dissemination of antigen throughout the lymphatic system may explain in part why the presentation of the antigen using the methods described herein ultimately results in long lasting cellular and humoral immunity.

The phrase “pharmaceutically acceptable” refers to molecular entities and compositions that are physiologically tolerable and do not typically produce an allergic or similar untoward reaction, such as gastric upset, dizziness and the like, when administered to a human. In a specific embodiment, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host.

The term “immunogenic” refers to the ability of an antigen to elicit an immune response, either humoral or cell mediated. An “immunogenically effective amount” as used herein refers to the amount of antigen sufficient to elicit an immune response, either a cellular (T cell) or humoral (B cell or antibody) response, as measured by standard assays known to one skilled in the art. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum, or by measuring the number of antigen specific colony forming units in the spleen. Furthermore, the level of protection of the immune response may be measured by challenging the immunized host with the antigen that has been injected. For example, if the antigen to which an immune response is desired is a virus or a tumor cell, the level of protection induced by the “immunogenically effective amount” of the antigen is measured by detecting the level of survival after virus or tumor cell challenge of the animals.

The term “mucosal immunity” refers to resistance to infection across the mucous membranes. Mucosal immunity depends on immune cells and antibodies present in the linings of reproductive tract, gastrointestinal tract and other moist surfaces of the body exposed to the outside world. Thus, a person having mucosal immunity is not susceptible to the pathogenic effects of foreign microorganisms or antigenic substances as a result of antibody secretions of the mucous membranes. Mucosal epithelia in the gastrointestinal, respiratory, and reproductive tracts produce a form of IgA (IgA, secretory) that serves to protect these ports of entry into the body. Since many pathogens enter the host by way of the mucosal surfaces, a vaccine that elicits mucosal immunity would be beneficial in terms of protection from many known pathogens, such as influenza or SARS virus.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5N to 3N direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55E, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). Preferably a minimum length for a hybridizable nucleic acid is at least about 10 nucleotides; more preferably at least about 15 nucleotides; most preferably the length is at least about 20 nucleotides.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5N (amino) terminus and a translation stop codon at the 3N (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3N to the coding sequence.

“Expression control sequences”, e.g., transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3N direction) coding sequence. For purposes of defining the present invention, the promoter sequence is bounded at its 3N terminus by the transcription initiation site and extends upstream (SN direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control of” or “operatively associated with” or “operably linked” a transcriptional and translational control sequence in a cell when RNA polymerase transcribes the coding sequence into mRNA, which is then trans-RNA spliced and translated into the protein encoded by the coding sequence.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667).

Two DNA sequences are “substantially homologous” or “substantially similar” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, two amino acid sequences are “substantially homologous” or “substantially similar” when greater than 70% of the amino acids are identical, or functionally identical. Preferably, the similar or homologous sequences are identified by alignment using, for example, the GCG (Genetics Computer Group, Program Manual for the GCG Package, Version 7, Madison, Wis.) pileup program.

A “vector” is a DNA molecule, capable of replication in a host organism, into which a gene is inserted to construct a recombinant DNA molecule.

“Long term protection”, as used herein, refers to the protection from disease or infection, which is obtained from vaccination with an antigen, and which may last for several months to years following immunization. Thus, even after several months to years following vaccination, an individual who has been vaccinated may have “long term protection” from infection, and would not be susceptible to infection upon challenge with or exposure to the pathogen from which the vaccine antigen was obtained.

“Virus-like particles” or VLPs as used herein, are membrane-surrounded structures comprising at least one viral surface protein embedded within the membrane of the host cell in which the VLPs are produced. VLPs do not contain intact viral nucleic acid and are, therefore, non-infectious. Exemplary VLPs of the invention include influenza virus-like particles, including influenza VLPs. Preferably, there is sufficient viral surface protein on the surface of the VLP so that when a VLP preparation is formulated into an immunogenic composition and administered to an animal or human, an immune response (cell-mediated and/or humoral) is elicited. The viral surface protein may be a full length polypeptide, or a truncate, variant, modified polypeptide thereof. Such polypeptides should retain at least one surface antigenic determinant against which an immune response may be generated, preferably a protective immune response.

“Subunit vaccines” are cell-free vaccine prepared from purified antigenic components of pathogenic microorganisms, thus carrying less risk of adverse reactions than whole-cell preparations. These vaccines are made from purified proteins or polysaccharides derived from bacteria or viruses. They include such components as toxins and cell surface molecules involved in attachment or invasion of the pathogen to the host cell. These isolated proteins act as target proteins/antigens against which an immune response may be mounted. The proteins selected for a subunit vaccine are normally displayed on the cell surface of the pathogen, such that when the subject's immune system is subsequently challenged by the pathogen, it recognizes and mounts an immune reaction to the cell surface protein and, by extension, the attached pathogen. Because subunit vaccines are not whole infective agents, they are incapable of becoming infective. Thus, they present no risk of undesirable virulent infectivity, a significant drawback associated with other types of vaccines. Subunit molecules from two or more pathogens are often mixed together to form combination vaccines. The advantages to combination vaccines is that they are generally less expensive, require fewer inoculations, and, therefore, are less traumatic to the animal.

A “DNA vaccine” relates to the use of genetic material (e.g., nucleic acid sequences) as immunizing agents. In one aspect, the present invention relates to the introduction of exogenous or foreign DNA molecules into an individual's tissues or cells, wherein these molecules encode an exogenous protein capable of eliciting an immune response to the protein. The exogenous nucleic acid sequences may be introduced alone or in the context of an expression vector wherein the sequences are operably linked to promoters and/or enhancers capable of regulating the expression of the encoded proteins. The introduction of exogenous nucleic acid sequences may be performed in the presence of a cell stimulating agent capable of enhancing the uptake or incorporation of the nucleic acid sequences into a cell. Such exogenous nucleic acid sequences may be administered in a composition comprising a biologically compatible or pharmaceutically acceptable carrier. The exogenous nucleic acid sequences may be administered by a variety of means, as described herein, and well known in the art. The DNA is linked to regulatory elements necessary for expression in the cells of the individual. Regulatory elements include a promoter and a polyadenylation signal. Other elements known to skilled artisans may also be included in genetic constructs of the invention, depending on the application. The following references pertain to methods for the direct introduction of nucleic acid sequences into a living animal: Nabel et al., (1990) Science 249:1285-1288; Wolfe et al., (1990) Science 247:1465-1468; Acsadi et al. (1991) Nature 352:815-818; Wolfe et al. (1991) BioTechniques 11(4):474-485; and Felgner and Rhodes, (1991) Nature 349:351-352, which are incorporated herein by reference. Such methods may be used to elicit immunity to a pathogen, absent the risk of infecting an individual with the pathogen. The present invention may be practiced using procedures known in the art, such as those described in PCT International Application Number PCT/US90/01515, wherein methods for immunizing an individual against pathogen infection by directly injecting polynucleotides into the individual's cells in a single step procedure are presented, and in U.S. Pat. Nos. 6,635,624; 6,586,409; 6,413,942; 6,406,705; 6,383,496.

As used herein, the term “genetic construct” refers to the DNA or RNA molecule that comprises a nucleotide sequence which encodes the target protein and which includes initiation and termination signals operably linked to regulatory elements including a promoter and polyadenylation signal capable of directing expression in the cells of the vaccinated individual. As used herein, the term “expressible form” refers to gene constructs which contain the necessary regulatory elements operably linked to a coding sequence of a target protein, such that when present in the cell of the individual, the coding sequence is expressed. As used herein, the term “genetic vaccine” refers to a pharmaceutical preparation that comprises a genetic construct.

The term “chimeric polypeptide” generally refers to a polypeptide comprising amino acid sequences obtained from at least two different proteins. In the case of the present invention, the chimeric polypeptide comprises the anti-DEC-205 antibody or fragments thereof and the amino acid sequence of a preselected antigen for which immunity or tolerance is desired. The chimeric polypeptide may further comprise a protein for a dendritic cell maturation factor. The recombinantly produced anti-DEC-205 antibody is a hybrid molecule, which has been genetically modified to contain both the antibody sequence, or fragments thereof, and also the antigen sequence and optionally the dendritic cell maturation sequence, and may be considered a chimeric polypeptide or fusion protein. For purposes of this invention the terms “chimeric polypeptide” may be used interchangeably with “fusion protein” or “fusion polypeptide”.

General Description

The inventors herein have found that enhanced and highly efficient antigen delivery to antigen-presenting cells may be achieved by targeting the antigen to a dendritic cell (DC)-restricted endocytic receptor, such as DEC-205, the asialoglycoprotein receptor, the Fcγ receptor, the macrophage mannose receptor, and Langerin. Furthermore, manipulating the environment of the thus-targeted antigen-presenting cell with regard to dendritic cell maturation can dictate the outcome of the endocytic receptor-targeted enhanced antigen presentation: towards eliciting a potent cellular immune response, or, alternatively, tolerance of the immune system to the endocytic receptor-targeted antigen. A preferred antigen-presenting cell is a dendritic cell (DC), and a preferred endocytic receptor is DEC-205. As will be seen below, the antigen may be targeted to the DEC-205 receptor on dendritic cells by any of a number of means, such as by conjugating or complexing the antigen to a DEC-205 ligand such as an antibody to DEC-205, or utilizing a fusion protein which is a hybrid of an anti-DEC-205 antibody and the antigen, if the antigen is a protein or peptide. Both exposure to the targeted antigen and manipulation of DC maturation in the environment of the antigen-presenting cell may be independently performed ex vivo or in vivo. Manipulation of DC maturation includes exposing or not exposing the antigen-presenting cells to a CD maturation stimulus such as a CD40 ligation promoting agent(s), or exposing the antigen-presenting cells to an agent which abrogates a DC maturation stimulus such as CD40 ligation, the latter in order to achieve an environment in which DC maturation does not occur.

Dendritic cells (DCs) have the capacity to initiate immune responses, but it has been postulated that they may also be involved in inducing peripheral tolerance. As will be seen in the examples below, to examine the function of DCs in the steady state, the present inventors devised an antigen delivery system targeting these specialized antigen presenting cells in vivo using a monoclonal antibody to the DC-restricted endocytic receptor, DEC-205. The results show that this route of antigen delivery to DCs is several orders of magnitude more efficient than free peptide in Complete Freund's Adjuvant (CFA) in inducing T cell activation and cell division. However, T cells activated by antigen delivered to DCs in this fashion without more are not polarized to produce Th1 cytokine IFN-γ and the activation response is not sustained. Within 7 days the number of antigen-specific T cells is severely reduced, and the residual T cells become unresponsive to systemic challenge with antigen in CFA. Thus, without dendritic cell stimulation at the time of antigen presentation, tolerance to the delivered antigen rather than induction of a cellular immune response is achieved.

In contrast, co-injection of the DC-targeted antigen with anti-CD40 agonistic antibody changes the outcome from tolerance to prolonged T cell activation and immunity. This targeting enables a study of the consequences of antigen presentation by DCs in naive mice with a polyclonal T cell repertoire. The inventors of the present application have provided for novel methods for promoting highly efficient antigen presentation, which results in robust and long lasting immune responses. Unexpectedly, the inventors have found that a single low subcutaneous dose of a protein-based vaccine was able to charge DCs with antigen systemically and for long periods, particularly on MHC class I products. In parallel, the mice developed immunity, including CD8⁺ T cell mediated immunity, which was considerably enhanced relative to prior methods of immunization with 1000 fold higher doses of antigen and was associated with stronger protection in anti-viral and anti-tumor models. More importantly, the inventors have identified a means of generating long lasting cellular immunity against non-replicating antigens, and have thus provided methods of generating T cell responses that mimic those seen in individuals that have experienced an active infection. These methods have provided for novel improvements in strategies for vaccine development, or alternatively, for improved methods for inducing tolerance against antigens for which an immune response is not desired.

While co-pending application Ser. No. 09/586,704 exploited the restriction of the DEC-205 receptor molecule to dendritic cells as a means for targeted DC delivery, it was not appreciated until the studies described herein of the several orders of magnitude increased efficiency of antigen delivery by the DEC-205 route as compared to other routes of antigen delivery to dendritic cells, nor was it known that the induction of tolerance could be achieved by targeted delivery of an antigen through an endocytic receptor such as DEC-205 in concert with the absence of CD40 ligation. While the examples below are focused on DEC-205 as the DC receptor for targeting and enhanced uptake thereof, other DC endocytic receptors such as the asialoglycoprotein receptor, the Fc.gamma. receptor, the macrophage mannose receptor, and Langerin, are embraced herein, and all utilities of DEC-205 are applicable to this as well as other endocytic receptors. Moreover, while enhanced antigen presentation by antigen-presenting cells to T cells is a desirable goal achieved herein, enhanced targeting to DC of any substance or molecule is embraced herein, such as enhanced genetic manipulation of DC by targeting a polynucleotide thereto for genetic modification including transfection or antisense therapy, or for delivery of a DNA vaccine and the like. These other aspects of the invention are fully embraced herein.

Exposing antigen-presenting cells to the DEC-205-targeted antigen and any of the foregoing DC maturation stimuli or maturation-inhibiting factors may be achieved in a variety of ways, for example, by exposing isolated antigen-presenting cells ex vivo to the targeted antigen before returning them to the animal, and then no administration to the animal of any factors, or administration of a DC maturation factor, such as, in the case of CD40 ligation, of an anti-CD40 agonistic antibody, or administration to the animal of a factor that will inhibit CD40 ligation in vivo. Alternatively, both antigen exposure and manipulation of CD40 ligation may be performed ex vivo before the antigen-presenting cells are optionally isolated and then readministered to the animal. These and other variations in the protocols are fully embraced by the invention herein, which in this embodiment essentially combines DEC-205-targeted antigen delivery with manipulation of CD40 ligation to modulate the immune response to the antigen. As noted above, the combination of any other endocytic receptor-binding molecule and any other DC maturation stimulus or factor to achieve an enhanced immune response is fully embraced by the teachings herein.

Various routes of delivery are contemplated for an in-vivo administered therapy as described herein. One of the purposes of DC delivery plus DC maturation is to enhance an immune response to a particular antigen, and the methods of the invention achieve such a goal by a vaccination protocol using an immunogen conjugated to a DC-targeted molecule, and co-administration of a DC maturation stimulus, is described herein. Such conjugates, as well as DC maturation stimuli (or inhibitors thereof for the induction of tolerance), may be delivered to the body by any appropriate route for the particular antigen involved. Such routes may include administration parenterally, transmucosally, e.g., orally, intranasally, or rectally, or transdermally. Parenteral administration includes intravenous injection, intra-arterial, intramuscular, intradermal, subcutaneous, intraperitoneal, intraventricular, and intracranial administration. Pulmonary delivery is also embraced, as are means for achieving delivery across the blood brain barrier. Intra-intestinal immunization may be achieved by delivery to the immune cells of the intestinal tract. Various formulations of the conjugate, including sustained release formulations, in order to achieve the optimal immunization protocol for the intended goal of the immunogen, are fully embraced herein. Targeting the conjugate on DEC-205 on brain endothelium is another means for achieving the delivery of the antigen across the blood brain barrier.

DEC-205 is described in co-pending application Ser. No. 09/586,704, and incorporated herein by reference in its entirety. Any means for targeting an antigen or antigenic fragment thereof to the DEC-205 receptor on dendritic or other antigen-presenting cells is embraced by the present invention. For example, an antibody to DEC-205 may be used, and the antigen or antigenic fragment thereof conjugated to the antibody using a cross-linking agent. In another embodiment, the antigen or fragment thereof may be part of a chimeric or fusion polypeptide comprising the antibody to DEC-205, wherein a polynucleotide encoding both the antibody to DEC-205 and the fragment reside on the same polynucleotide construct, and are expressed in the form of the chimeric, single-chain antibody-antigen. The antigen may be located at any site in the antibody where it does not interfere with the targeting of the chimeric antibody-antigen to the DEC-205; by way of non-limiting example, appending the antigen to the C-terminus of the antibody heavy chain achieves this purpose. In another embodiment, a DEC-205 targeted composition of the invention may comprise a protein or peptide DEC-205 ligand other than an antibody, and a protein or peptide antigen, residing on the same polypeptide chain. Polynucleotides encoding the aforementioned chimeric polypeptide are also embraced herein.

One non-limiting example of a monoclonal antibody to DEC-205 that may be used in the present invention is NLDC-145, as described in G. Kraal, M. Breel, M. Janse, G. Bruin, J Exp Med 163, 981-97 (1986). However, the invention is not so limited and any antibody may be used, directed to the DEC-205 of the species of animal in which immune therapy by the methods herein is to be achieved. Preferably, the DEC-205-binding molecule binds to human DEC-205.

In another embodiment, a bispecific antibody may be provided, one antigen-binding site directed to DEC-205, and the other antigen-binding site directed to the antigen selected for manipulation of the immune response. This embodiment is particularly useful if an endogenous antigen, such as a cancer antigen, is desirably chosen for enhancing an immune response thereto: administration of the bispecific antibody to the patient exhibiting circulating levels of the cancer antigen will target it to the dendritic cells, which, in combination with the manipulation of CD40 ligation as described herein, will result in an enhanced anti-cancer antigen immune response.

In a further embodiment, if any antibody method is used for the targeting of the antigen to DEC-205, binding of the antibody to the Fc receptor is desirably minimized. To minimize such binding, a recombinant antibody used herein may be modified such as to alter the Fc region of the antibody molecule to reduce its recognition by the Fc receptor. Such modifications have been described (R. A. Clynes, T. L. Towers, L. G. Presta, J. V. Ravetch, Nat Med 6, 443-6 (2000)), and this and other modifications of the conjugate of chimeric DEC-205-binding molecule and the antigen to increase its specificity for binding to the DEC-205 receptor are full embraced herein.

Natural ligands for DEC-205 or the other endocytic receptors described herein may also be used as an alternative to an antibody to the receptor to enhance the delivery of an associated antigen. Other ligands may be identified as described in co-pending application Ser. Nos. 09/586,704, 08/381,528, as well as in PCT/US96/01383 (WO9623882).

Exploitation of the antigen-presenting cell endocytic receptor for enhanced antigen delivery, with or without subsequent manipulation of DC maturation for modulation of an immune response, may be utilized for antigen delivery and modulation of an immune response in any mammalian species, preferably human but not so limiting, and may be used in non-human primates, livestock and companion animals, zoo animals, as well as animals in the wild. Vaccination by the methods and using the agents herein of domestic or livestock animals against pathogens such as foot and mouth disease, rabies, distemper, among a large number of important pathogens and parasites, is fully embraced herein. Vaccination of humans against viral, bacterial, protistan and multicellular parasitic diseases is also fully embraced herein, including but not limited to human immunodeficiency virus, human papillomavirus, Epstein-Barr virus, herpes simplex virus, measles virus, smallpox virus, chicken pox virus, the various hepatitis viruses, rubella virus, mumps virus, influenza virus, SARS virus, infectious bacterial agents including pneumococci, tuberculosis, Yersinia pestis, Borrelia burgdorferi, the causative agent of Lyme disease, and diphtheria, among others. Protistan antigens include malaria and trypanosomatids. Multicellular parasites include schistosomes, roundworms, and others. The foregoing are merely non-limiting examples of antigens and diseases associated therewith, and the invention herein embraces all such antigens for the purposes described.

The selection of antigen for enhanced DC delivery and modulation of the immune response thereto may be any antigen for which either an enhanced immune response is desirable, or for which tolerance of the immune system to the antigen is desired. In the case of a desired enhanced immune response to a particular antigen, antigens such as infectious disease antigens for which a protective immune response may be elicited are exemplary. For example, the antigens from HIV under consideration are gag, env, pol, tat, rev, nef protein, reverse transcriptase, and other HIV components. The E6 and E7 proteins from human papilloma virus are also under consideration. Furthermore, the EBNA1 antigen from herpes simplex virus is also under consideration. Other viral antigens for consideration are hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens. In addition, the F1 and V proteins from Yersinia pestis are under consideration, as are the malaria circumsporozoite proteins. Other bacterial antigens which can be used in the compositions and methods of the invention include, but are not limited to, pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as romps and other rickettsiae bacterial antigen components. Also included with the bacterial antigens described herein are any other bacterial, mycobacterial, mycoplasmal, rickettsial, or chlamydial antigens. Examples of protozoa and other parasitic antigens include, but are not limited to, plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components. In addition to the infectious and parasitic agents mentioned above, another area for desirable enhanced immunogenicity to a non-infectious agent is in the area of dysproliferative diseases, including but not limited to cancer, in which cells expressing cancer antigens are desirably eliminated from the body. Tumor antigens which can be used in the compositions and methods of the invention include, but are not limited to, prostate specific antigen (PSA), breast, ovarian, testicular, melanoma, telomerase; multidrug resistance proteins such as P-glycoprotein; MAGE-1, alpha fetoprotein, carcinoembryonic antigen, mutant p53, papillomavirus antigens, gangliosides or other carbohydrate-containing components of melanoma or other tumor cells. It is contemplated by the invention that antigens from any type of tumor cell can be used in the compositions and methods described herein. The antigen conjugated or coupled to an endocytic receptor-binding molecule may be a cancer cell, or immunogenic materials isolated from a cancer cell, such as membrane proteins. Included are survivin and telomerase universal antigens and the MAGE family of cancer testis antigens. Antigens which have been shown to be involved in autoimmunity and could be used in the methods of the present invention to induce tolerance include, but are not limited to, myelin basic protein, myelin oligodendrocyte glycoprotein and proteolipid protein of multiple sclerosis and CII collagen protein of rheumatoid arthritis.

The antigen may be a portion of an infectious agent such as HZV-1, EBV, HBV, influenza virus, SARS virus, poxviruses, malaria, or HSV, by way of non-limiting examples, for which vaccines that mobilize strong T-cell mediated immunity (via dendritic cells) are needed.

The antigen may be any molecule or substance for enhanced DC delivery, not only for the immunologic modulation purposes herein but additionally, for example, to promote or enhance the delivery of agents to dendritic cells. In one example, genetic manipulation of dendritic cells may be achieved by targeting a polynucleotide to a dendritic cell via an endocytic receptor such as DEC-205. The polynucleotide may be DNA, RNA, or an antisense oligonucleotide, by way of non-limiting examples. Such a procedure increases the amount of a molecule desirably introduced into a dendritic cell by taking advantage of the enhanced uptake when a molecule is associated with or conjugated to a ligand for or other means of targeting the molecule to DEC-205 or another endocytic receptor. Although the cell may be further manipulated after the delivery, such as maturation or lack thereof, the enhanced delivery aspect of the invention is not necessarily associated with any further manipulation of the dendritic cells. For example, the cells may be removed from the body, a conjugate exposed thereto to deliver the molecule, such as an antisense oligonucleotide or a polynucleotide construct for gene therapy, and the dendritic cells reintroduced to the body. This example is merely illustrative of this aspect of the invention and is in no way limiting.

Attachment of the antigen, or other molecule desirably introduced into a dendritic cell, to the DEC-205- or other endocytic receptor-binding agent may be by any suitable means, including but not limited to covalent attachment by means of a bifunctional cross-linking reagent, and activation of one member and then cross-linking to a functional group on the other. Various cross-linking agents and functional group activating agents such as described from Pierce Chemical Co., Rockford, Ill., are useful for these purposes. In the instance wherein both the endocytic receptor-binding molecule and the antigen are proteins or peptides, they may be expressed on a single polypeptide chain, wherein the single polypeptide chain retains the endocytic receptor-binding activity and the protein or peptide antigen retains its desired features. In one non-limiting example, the endocytic receptor-binding molecule is an DEC-205-binding molecule such as a monoclonal antibody to DEC-205, and one chain of the antibody and the antigen are provided in a recombinant polynucleotide construct in which the expressed polypeptide comprises both an antibody chain with a DEC-205 binding site, and the antigen.

In contrast to a desired enhanced immune response to an antigen, in many instances a lack of an immune response is desired to a particular antigen. By way of non-limiting example, an individual who is a candidate for a transplant from a non-identical twin may suffer from rejection of the engrafted cells, tissue or organ, as the engrafted antigens are foreign to the recipient. Prior tolerance of the recipient individual to the intended engraft abrogates or reduces later rejection. Reduction or elimination of chronic anti-rejection therapies is achieved by the practice of the present invention. In another example, many autoimmune diseases are characterized by a cellular immune response to an endogenous or self antigen. Tolerance of the immune system to the endogenous antigen is desirable to control the disease. In a further example, sensitization of an individual to an industrial pollutant or chemical, such as may be encountered on-the-job, presents a hazard of an immune response. Prior tolerance of the individual's immune system to the chemical, in paiticular in the form of the chemical reacted with the individual's endogenous proteins, may be desirable to prevent the later occupational development of an immune response. Allergens are other antigens for which tolerance of the immune response thereto is desirable. Likewise, autoantigens could be delivered to dendritic cells by a way that elicits specific immunotolerance.

The invention is directed not only to the use of the aforementioned DEC-205-binding molecules such as anti-DEC-205 antibody conjugates or fusion proteins comprising an antigen, but also to compositions comprising such conjugates of chimeric proteins, and pharmaceutical compositions comprising them, for vaccination or other immune modulation of an animal, preferably a human but any mammalian animal. It also embraces polynucleotide sequences encoding chimeric or single-chain polypeptides comprising an antigen-presenting cell endocytic receptor-binding molecule, such as a DEC-205-binding molecule, and an antigen, The DEC-205-binding molecule may be an antibody, a DEC-205-binding protein, a lectin, or any DEC-205-binding fragment of any of the foregoing.

Alternatively, non-antibody means for targeting an antigen to an endocytic receptor such as DEC-205 may be used, such as those described in co-pending application Ser. No. 09/586,704. Such targeting molecules include a carbohydrate ligand, such as a glycan, that binds to DEC-205, in particular to one of its lectin domains. DEC-205 is known to possess about ten C-type lectin domains, and any or a combination of these domains may serve as targets for specific binding of an antigen to DEC-205. Moreover, other dendritic cell endocytic receptors other than DEC-205, such as but not limited to the asialoglycoprotein receptor, the Fcy receptor, the macrophage mannose receptor, and Langerin, may be used in a likewise fashion as DEC-205 described herein.

In concert with delivery of the antigen to DEC-205 on the antigen-presenting cell, a DC maturation stimulus or inhibition thereof, such as is achieved by manipulation of CD40 ligation of the antigen-presenting cell, is desirable to achieve the desired immune response outcome. As mentioned above, in concert with CD40 ligation, a robust cellular immune response toward the antigen is achieved. In the absence of CD40 ligation, tolerance to the antigen is achieved. The present invention embraces all such manipulations of CD40 ligation in concert with DEC-205 antigen targeting for the purposes herein. Moreover, the combination of any DC maturation signal and any endocytic receptor-targeted antigen delivery is embraced by the present invention.

DC maturation may be achieved by any one of a number of means, or combinations thereof. Such maturation signals may be achieved by, for example, CD40 ligation, CpG, oligodeoxyribonucleotides, ligation of the IL-1, TNFα. or TOLL-like receptor, bacterial lipoglycans and polysaccharides or activation of an intracellular pathway such as TRAF-6 or NFκB. These are merely illustrative and one of skill in the art will be aware of other means for inducing DC maturation, all of which are embraced herein in combination with endocytic receptor delivery of a preselected antigen.

In a preferred but non-limiting embodiment, CD40 ligation may be achieved using any of a number of methods. Exposure of the antigen-presenting cell to an agonistic anti-CD40 antibody achieves CD40 ligation. An antibody such as but not limited to FGK 45 described herein may be used. The invention embraces polyclonal antibodies, monoclonal antibodies, chimeric antibodies, antibody fragments such as F(ab) fragments, and any antibody fragments or recombinant antibody fragments or constructs comprising an antigen-binding site. CD40L or a CD40-binding fragment thereof may be used, such as described in C. Caux, et al., J Exp Med 180, 1263-72 (1994); K. Inaba, et al., J Exp Med 191, 927-36 (2000) and F. Sallusto, A. Lanzavecchia, J Exp Med 179, 1109-18 (1994), by way of non-limiting examples. Ligands that signal Toll like receptors, e.g., CpG oligodeoxynucleotides, RNA, bacterial lipoglycans and polysaccharides, TNF receptors such as the TNFα. receptor, IL-1 receptors, and compounds that activate TRAF 6 and NF-κB signaling pathways, may be used.

As mentioned above, to achieve an enhanced immune response, a DC maturation stimulus such as CD40 ligation is desired, as may be achieved by exposing the antigen-presenting cells ex vivo or in vivo to an aforementioned DC maturation signal. In contrast, to tolerize the animal to a DEC-205-targeted antigen, the absence of DC maturation is necessary. This may be achieved ex vivo or in vivo. Agents that block DC maturation signals such as CD40 ligation may be used, such as but not limited to an antibody to CD40L, the TNF-family member that is expressed on activated CD4 T cells, platelets and mast cells, or a soluble CD40 or fragment thereof capable of binding CD40L and inhibiting dendritic cell maturation. Blockage of any of the DC maturation signals mentioned throughout herein, which are merely exemplary, may be performed in concert with endocytic receptor-mediated antigen delivery to achieve the desired tolerance to the antigen. Other means of preventing or inhibiting DC maturation are fully embraced herein.

The methods of the invention may be carried out ex vivo or in vivo, and independently with regard to antigen targeting to an endocytic receptor, such as DEC-205 in the following examples, and manipulation of DC maturation, such as by CD40 ligation manipulation, in the following examples. For fully ex vivo methods, dendritic cells may be isolated from whole blood of an individual, and exposed ex vivo both to the DEC-205-targeted antigen and to CD40 ligation, or in the absence of CD40 ligation, before the dendritic cells are optionally isolated and then readministered to the individual. In another embodiment, isolated dendritic cells are exposed to DEC-205-targeted antigen and then optionally isolated before administration to the individual.

Subsequent to readministration, CD40 ligation is manipulated, for example, by no additional steps (to induce tolerance), by administration of a CD40 ligation promoting agent(s) such as an agonistic anti-CD40 antibody for enhancing the development of a cellular response, or for tolerance, a CD40 ligation inhibiting agent, as mentioned above. Routes of in-vivo administration are described herein.

In vivo methods are also included, wherein the DEC-205-targetet antigen is administered to the individual, such as in the form of a vaccine, and then CD40 ligation is manipulated in vivo, by any of the foregoing methods. Route of administration of the vaccine are as described above. Administration of a DC maturation signal may also be performed in vivo, systemically or locally, and via any suitable route of administration.

As mentioned above, the present invention embraces DEC-205-targeted antigen compositions, such as but not limited to a chimeric anti-DEC-205 antibody comprising an antigen, or a conjugate of an aforementioned antibody and an antigen. It is further directed to other DC endocytic receptor-targeted antigens, such as an antigen conjugated to an asialoglycoprotein receptor-targeted molecule.

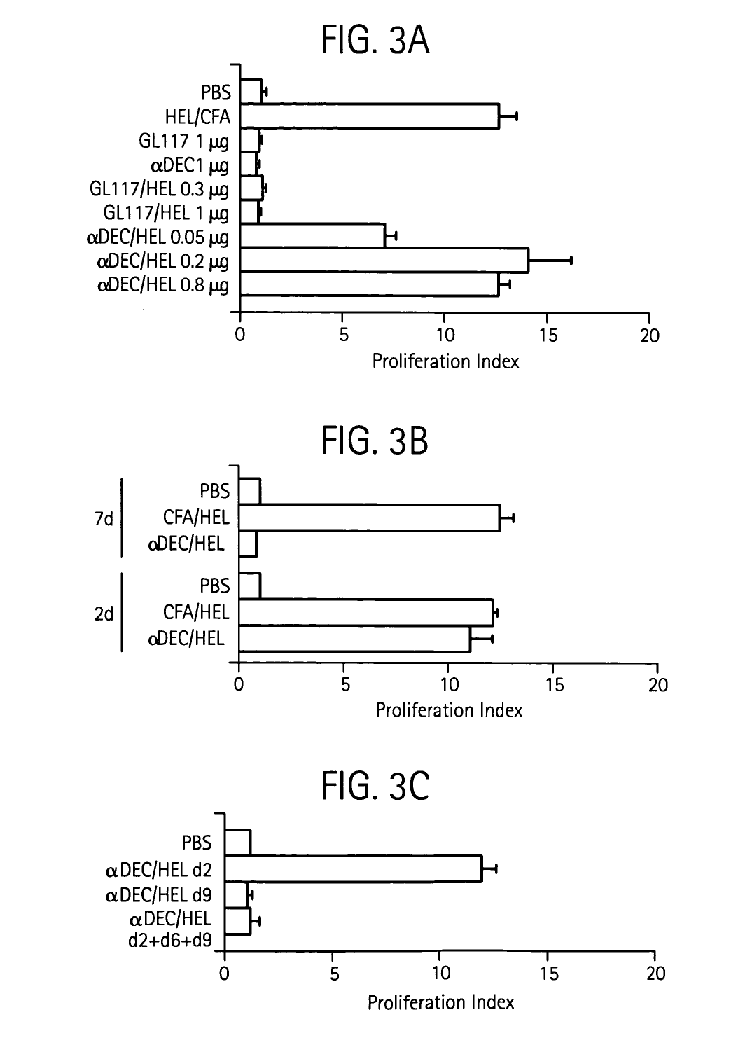

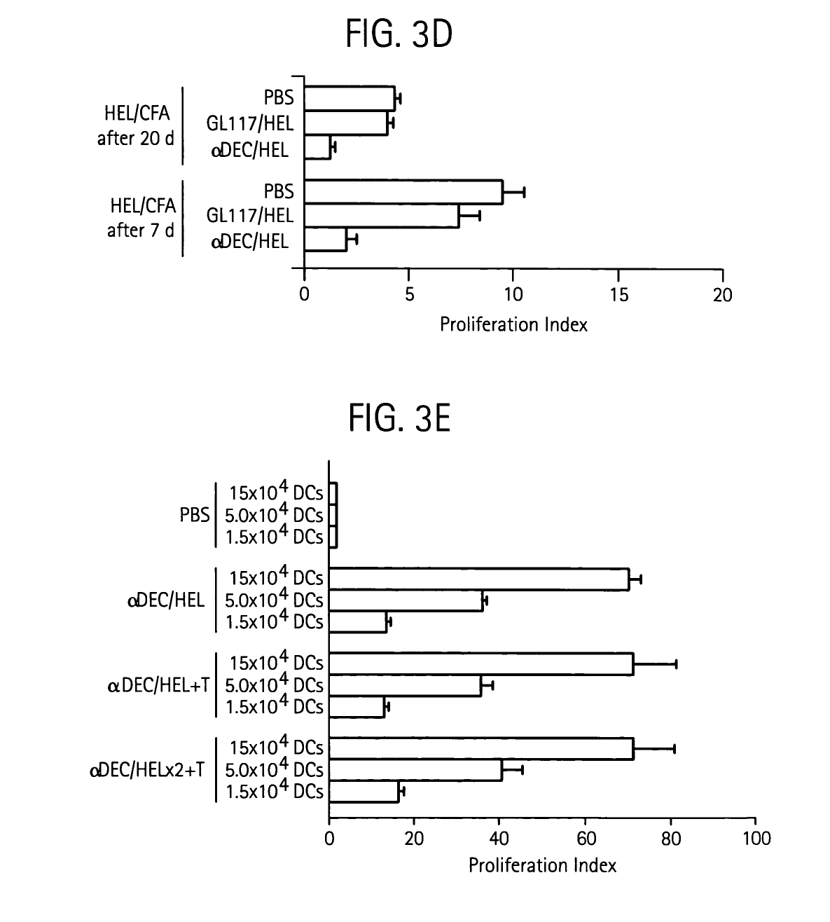

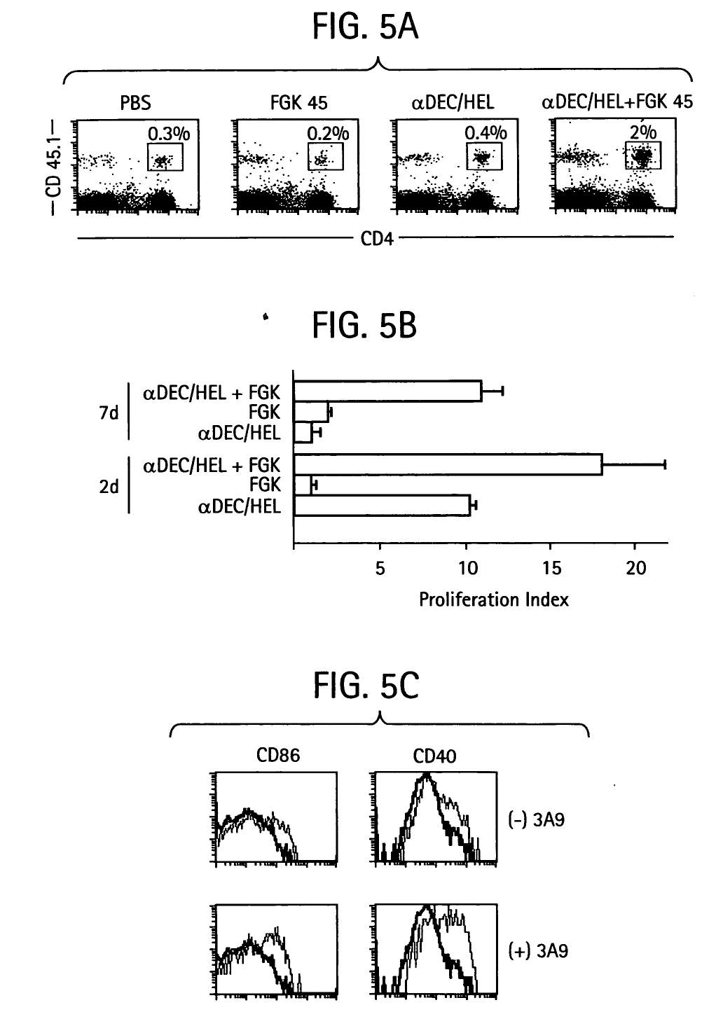

As will be shown in the examples below, manipulation of the environment of the antigen-presenting cell governs whether a tolerance or induced immune response is achieved. When DCs are charged with antigen in the steady state, these MHC II-rich cells do not induce normal Th-subset polarization or prolonged T cell expansion and activation. Instead, the T cells exposed to antigen on DCs in vivo either disappear or become anergic to antigenic re-stimulation. This indicates that in the steady state, the primary function of DCs is to maintain peripheral tolerance (see FIGS. 3C and 3D). Indeed, combined administration of DC-targeted antigen with an agonistic anti-CD40 antibody that up-regulates co-stimulatory molecules like CD86 on the surface of DCs (see FIG. 5C), prevents induction of peripheral tolerance and leads to prolonged T cell activation.

Furthermore, it will be shown that a covalent complex between an antigen, e.g., ovalbumin, and an anti-DEC-205 antibody efficiently targets the MHC I pathway and leads to profound tolerance of CD8 T cells to the antigen.

Genes Encoding DEC, or Fragments, Derivatives, Chimeras, or Analogs Thereof

The present invention contemplates the use of antibodies having specificity for DEC-205 for enhanced delivery of antigens to dendritic cells bearing this endocytic receptor for either enhancement of immune responses or for induction of tolerance to specific antigens. Enhanced immunity, in particular cellular immunity, is achieved through the antigen/anti-DEC-205 antibody complex delivered with a dendritic cell maturation factor. In the event that tolerance induction is desired, the antigen/anti-DEC-205 antibody complex is delivered without the dendritic cell maturation factor.

Co-pending application Ser. No. 09/586,704, to which the present application claims priority, describes the endocytic cell membrane receptor DEC-205, which is present on mammalian dendritic cells as well as on certain other cell types, and describes its role in antigen processing, and exploiting the existence of DEC-205 primarily on dendritic cells for targeting antigens for uptake and presentation by dendritic cells. The application describes ligands of DEC-205, such as antibodies, carbohydrates as well as other DEC-205-binding agents for targeting antigens to DEC-205 and thus specifically to dendritic cells. Furthermore, the parent application relates to isolation and cloning of human DEC, which is further characterized by having a carboxyl-terminal sequence RHRLHLAGFSSVRYAQGVNEDE[MLPSFHD (SEQ ID NO: 1), and characterized by binding to a rabbit polyclonal antibody raised against full length murine DEC-205, but not reacting with monoclonal antibody NLDC-145. “DEC” is defined as an integral membrane protein found primarily on dendritic cells, B cells, brain capillaries, bone marrow stroma, epithelia of intestinal villi, and pulmonary airways, as well as cortical epithelium of the thymus and dendritic cells in the T cell areas of peripheral lymphoid organs. Moreover, the protein has been found predominantly on Dendritic cells and thymic Epithelial Cells, and has a molecular weight of 205 kDa, thus it has been termed DEC-205. The sequences to follow for murine and human DEC-205 can be found in PCT/US96/01383 and U.S. Ser. No. 08/381,528, to which the present application claims priority. The nucleic acid sequence for human DEC-205 can be found in SEQ ID NO: 5, and the protein sequence in SEQ ID NO: 6. The N terminal sequence for human DEC-205 can be found in SEQ ID NO: 2. The murine DEC-205 protein sequence can be found in SEQ ID NO: 3 and the C terminal murine DEC-205 sequence in SEQ ID NO: 4.

Furthermore, in specific embodiments in the parent application, a specific nucleotide sequence of a human DEC-encoding DNA is provided (SEQ ID NO:5). Also provided are deduced coding sequences for both murine and human DEC polypeptides having the amino acid sequences depicted in SEQ ID NO: 3 and SEQ ID NO: 6, respectively. It is contemplated herein that these sequences may be used in the preparation of antibodies to human or murine DEC-205 for practice of the methods described in the present application.

Accordingly, in the present application, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A gene encoding DEC, whether genomic DNA or cDNA, can be isolated from any source, particularly from a human cDNA or genomic library. Methods for obtaining DEC gene are well known in the art, as described above (see, e.g., Sambrook et al., 1989, supra). In specific embodiment, infra, a cDNA encoding murine DEC-205 is isolated from a dendritic cell library. In addition, probes derived from the murine gene were used to isolate the corresponding human dec cDNA and the murine genomic dec gene.

Accordingly, any animal cell potentially can serve as the nucleic acid source for the molecular cloning of a dec gene. The DNA may be obtained by standard procedures known in the art from cloned DNA (e.g., a DNA “library”), and preferably is obtained from a cDNA library prepared from tissues with high level expression of the protein (e.g., a dendritic cell cDNA or thymic epithelial cDNA library, since these are the cells that evidence highest levels of expression of DEC), by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography. Once the DNA fragments are generated, identification of the specific DNA fragment containing the desired dec gene may be accomplished in a number of ways. For example, if an amount of a portion of a dec gene or its specific RNA, or a fragment thereof, is available and can be purified and labeled, the generated DNA fragments may be screened by nucleic acid hybridization to the labeled probe (Benton and Davis, 1977, Science 196:180; Grunstein and Hogness, 1975, Proc. Natl. Acad. Sci. U.S.A. 72:3961). For example, a set of oligonucleotides corresponding to the partial amino acid sequence information obtained for the DEC protein can be prepared and used as probes for DNA encoding DEC, as was done in a specific example, infra, or as primers for cDNA or mRNA (e.g., in combination with a poly-T primer for RT-PCR). Preferably, a fragment is selected that is highly unique to DEC of the invention. Those DNA fragments with substantial homology to the probe will hybridize. As noted above, the greater the degree of homology, the more stringent hybridization conditions can be used. In a specific embodiment, the human dec cDNA was cloned using a 300 base-pair probe derived from the 3N coding sequence of murine dec cDNA. The human cDNA was obtained from a B lymphoma library, using high stringency hybridization conditions (0.1 SSC, 65EC). Thus, high stringency hybridization conditions are favored to identify a homologous dec gene from other species.

Further selection can be carried out on the basis of the properties of the gene, e.g., if the gene encodes a protein product having the isoelectric, electrophoretic, amino acid composition, or partial amino acid sequence of DEC protein as disclosed herein. Thus, the presence of the gene may be detected by assays based on the physical, chemical, or immunological properties of its expressed product. For example, cDNA clones, or DNA clones which hybrid-select the proper mRNAs, can be selected which produce a protein that, e.g., has similar or identical electrophoretic migration, isoelectric focusing or non-equilibriuin pH gel electrophoresis behavior, proteolytic digestion maps, or antigenic properties as known for DEC. For example, the rabbit polyclonal antibody to murine DEC, described in detail infra, can be used to confirm expression of DEC, both murine and human counterparts. In another aspect, a protein that has an apparent molecular weight of 205 kDa, and which is specifically digested to form a defined ladder (rather than a smear) of lower molecular weight bands, is a good candidate for DEC.

A dec gene of the invention can also be identified by mRNA selection, i.e., by nucleic acid hybridization followed by in vitro translation. In this procedure, nucleotide fragments are used to isolate complementary mRNAs by hybridization. Such DNA fragments may represent available, purified dec DNA, or may be synthetic oligonucleotides designed from the partial amino acid sequence information. Immunoprecipitation analysis or functional assays (e.g., tyrosine phosphatase activity) of the in vitro translation products of the products of the isolated mRNAs identifies the mRNA and, therefore, the complementary DNA fragments, that contain the desired sequences. In addition, specific mRNAs may be selected by adsorption of polysomes isolated from cells to immobilized antibodies specifically directed against DEC, such as the rabbit polyclonal anti-murine DEC antibody described herein.

A radiolabeled dec cDNA can be synthesized using the selected mRNA (from the adsorbed polysomes) as a template. The radiolabeled mRNA or cDNA may then be used as a probe to identify homologous dec DNA fragments from among other genomic DNA fragments.

The present invention also relates to cloning vectors containing genes encoding DEC antibodies, genes encoding antigens to be delivered to cells bearing receptors for DEC, and genes encoding dendritic cell maturation factors. These genes may be under the control of separate promoters or may all be under the control of one promoter.

The genes encoding DEC-205 or fragments thereof, antibodies to DEC-205, antigens to be delivered by said antibodies and dendritic cell maturation factors, such as, but not limited to agonistic anti-CD40 antibodies, can be produced by various methods known in the art. The manipulations which result in their production can occur at the gene or protein level. For example, the cloned DEC-205 or anti-DEC-205 antibody gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro.

Additionally, the DEC-205 or anti-DEC-205 antibody encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Preferably, such mutations enhance the functional activity of DEC or the anti-DEC antibody gene product. Alternatively, deletion mutants can be produced that encode fragments of the anti-DEC antibody (see Taylor et al., 1992, J. Biol. Chem. 267:1719). Any technique for mutagenesis known in the art can be used, including but not limited to, in vitro site-directed mutagenesis (Hutchinson, C., et al., 1978, J. Biol. Chem. 253:6551; Zoller and Smith, 1984, DNA 3:479-488; Oliphant et al., 1986, Gene 44:177; Hutchinson et al., 1986, Proc. Natl. Acad. Sci. U.S.A. 83:710), use of TAB7 linkers (Pharmacia), etc. PCR techniques are preferred for site directed mutagenesis (see Higuchi, 1989, “Using PCR to Engineer DNA”, in PCR Technology: Principles and Applicationsfor DNA Amplification, H. Erlich, ed., Stockton Press, Chapter 6, pp. 61-70).

The identified and isolated gene encoding DEC, or the genes encoding the anti-DEC antibody or fragments thereof, or the genes encoding the antigen or the dendritic cell maturation factor can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2μ plasmid.

In an alternative method, the desired gene may be identified and isolated after insertion into a suitable cloning vector in a “shot gun” approach. Enrichment for the desired gene, for example, by size fractionation, can be done before insertion into the cloning vector. Following expression of the gene product using standard techniques in the art, the gene product (ie. DEC-205) is purified and may be used in the preparation of antibodies for use in the methods of the present invention.

Antibodies to DEC

As noted above, and in accordance with the present invention, DEC produced recombinantly or by chemical synthesis, and fragments or other derivatives or analogs thereof, including fusion proteins, may be used as an immunogen to generate antibodies that recognize DEC. Such antibodies include but are not limited to polyclonal, monoclonal, chimeric, single chain, Fab fragments, and an Fab expression library. In a specific embodiment, infra, a rabbit polyclonal antibody is prepared against the N-terminal amino acid sequence of human and murine DEC-205. In another embodiment, a polyclonal antibody against intact, purified, human and murine DEC-205 was generated. In yet another embodiment, a recombinant fusion polypeptide is generated which contains the antibody sequence to DEC-205 and the protein sequence for the antigen on one polypeptide chain.

Various procedures known in the art may be used for the production of polyclonal antibodies to DEC-205, or derivatives or analogs thereof. For the production of antibody, various host animals can be immunized by injection with the non-allogeneic DEC-205, or a derivative (e.g., fragment or fusion protein) thereof, including but not limited to rabbits, mice, rats, sheep, goats, etc. In one embodiment, the DEC-205 or fragment thereof can be conjugated to an immunogenic carrier, e.g., bovine serum albumin (BSA) or keyhole limpet hemocyanin (KLH). Various adjuvants may be used to increase the immunological response, depending on the host species, as described above.

For preparation of monoclonal antibodies directed toward DEC-205, or fragment, analog, or derivative thereof, any technique that provides for the production of antibody molecules by continuous cell lines in culture may be used. These include but are not limited to the hybridoma technique originally developed by Kohler and Milstein (1975, Nature 256:495497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In general, the DEC-205 protein or fragments thereof is administered (e.g., intraperitoneal injection) to wild-type or inbred mice (e.g., BALB/c) or transgenic mice which produce desired antibodies, or rats, rabbits, chickens, sheep, goats, or other animal species which can produce native or human antibodies. The DEC-205 protein may be administered alone, or mixed with adjuvant. After the animal is boosted, for example, two or more times, the spleen or large lymph node, such as the popliteal in rat, is removed and splenocytes or lymphocytes are extracted and fused with myeloma cells using well-known processes, for example Kohler and Milstein ((1975) Nature 256:495497) and Harlow and Lane (Antibodies: A Laboratory Manual (Cold Spring Harbor Laboratory, New York (1988)). The resulting hybrid cells are then cloned in the conventional manner, e.g. using limiting dilution, and the resulting clones, which produce the desired monoclonal antibodies, are cultured. In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, J. Bacteriol. 159-870; Neuberger et al., 1984, Nature 312:604-608; Takeda et al., 1985, Nature 314:452454) by splicing the genes from a mouse antibody molecule specific for an DEC-205 together with genes from a human antibody molecule of appropriate biological activity can be used; such antibodies are within the scope of this invention. Furthermore, other chimeric polypeptides of the present invention are contemplated by splicing the genes from an anti-DEC antibody, either a murine or a human antibody, with genes encoding the antigen for which immunity or tolerance is desired. In addition, another chimeric polypeptide is envisioned whereby one can splice the genes encoding the anti-DEC-205 antibody with genes encoding the antigen and with genes encoding a dendritic cell maturation factor, such as but not limited to an anti-CD40 antibody, or an inflammatory cytokine.

According to the invention, techniques described for the production of single chain antibodies (U.S. Pat. No. 4,946,778) can be adapted to produce DEC-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for DEC-205, or its derivatives, or analogs.

Alternatively, antibodies against DEC-205 are derived by a phage display method. Methods of producing phage display antibodies are well-known in the art, for example, Huse, et al. ((1989) Science 246(4935): 1275-81).

Methods for identification and production of polynucleotides conferring a desired phenotype and/or encoding a protein, for example, an antibody, having an advantageous predetermined property which is selectable are known in the art (See for example, U.S. Pat. No. 6,576,467). Thus, in order to overcome many of the limitations in producing and identifying high-affinity immunoglobulins through antigen-stimulated B cell development (i.e., immunization and subsequent determination of the binding characteristics of the antibodies made), various prokaryotic expression systems are available which can be manipulated to produce combinatorial antibody libraries. Thereafter, these libraries may be screened for high-affinity antibodies to specific antigens, for example DEC-205. Recent advances in the expression of antibodies in Escherichia coli and bacteriophage systems have raised the possibility that virtually any specificity can be obtained by either cloning antibody genes from characterized hybridomas or by de novo selection using antibody gene libraries (e.g., from Ig cDNA).

Combinatorial libraries of antibodies have been generated in bacteriophage lambda expression systems which may be screened as bacteriophage plaques or as colonies of lysogens (Huse et al. (1989) Science 246: 1275; Caton and Koprowski (1990) Proc. Natl. Acad. Sci. (U.S.A.) 87: 6450; Mullinax et al (1990) Proc. Natl. Acad. Sci. (U.S.A.) 87: 8095; Persson et al. (1991) Proc. Natl. Acad. Sci. (U.S.A.) 88: 2432). Various embodiments of bacteriophage antibody display libraries and lambda phage expression libraries have been described (Kang et al. (1991) Proc. Natl. Acad. Sci. (U.S.A.) 88: 4363; Clackson et al. (1991) Nature 352: 624; McCafferty et al. (1990) Nature 348: 552; Burton et al. (1991) Proc. Natl. Acad. Sci. (U.S.A.) 88: 10134; Hoogenboom et al. (1991) Nucleic Acids Res. 19: 4133; Chang et al. (1991) J. Immunol. 147: 3610; Breitling et al. (1991) Gene 104: 147; Marks et al. (1991) J. Mol. Biol. 222: 581; Barbas et al. (1992) Proc. Natl. Acad. Sci. (U.S.A.) 89: 4457; Hawkins and Winter (1992) J. Immunol. 22: 867; Marks et al. (1992) Biotechnology 10: 779; Marks et al. (1992) J. Biol. Chem. 267: 16007; Lowman et al (1991) Biochemistry 30: 10832; Lerner et al. (1992) Science 258: 1313, incorporated herein by reference). Typically, a bacteriophage antibody display library is screened with an antigen (e.g., polypeptide, carbohydrate, glycoprotein, nucleic acid) that is immobilized (e.g., by covalent linkage to a chromatography resin to enrich for reactive phage by affinity chromatography) and/or labeled (e.g., to screen plaque or colony lifts).

One particularly advantageous approach has been the use of so-called single-chain fragment variable (scFv) libraries (Marks et al. (1992) Biotechnology 10: 779; Winter G and Milstein C (1991) Nature 349: 293; Clackson et al. (1991) op.cit.; Marks et al. (1991) J. Mol. Biol. 222: 581; Chaudhary et al. (1990) Proc. Natl. Acad. Sci. (USA) 87: 1066; Chiswell et al. (1992) TIBTECH 10: 80; McCafferty et al. (1990) op.cit.; and Huston et al. (1988) Proc. Natl. Acad. Sci. (USA) 85: 5879). Various embodiments of scFv libraries displayed on bacteriophage coat proteins have been described.

Beginning in 1988, single-chain analogues of Fv fragments and their fusion proteins have been reliably generated by antibody engineering methods. The first step generally involves obtaining the genes encoding V_{H} and V_{L} regions with desired binding properties; these V genes may be isolated from a specific hybridoma cell line, selected from a combinatorial V-gene library, or made by V gene synthesis. The single-chain Fv is formed by connecting the component V genes with an oligonucleotide that encodes an appropriately designed linker peptide, such as (Gly-Gly-Gly-Gly-Ser)₃ or equivalent linker peptide(s). The linker bridges the C-terminus of the first V region and N-terminus of the second, ordered as either V_{H}-linker-V_{L} or V_{L}-linker-V_{H}. In principle, the scFv binding site can faithfully replicate both the affinity and specificity of its parent antibody combining site.

Thus, scFv fragments are comprised of V_{H} and V_{L} regions linked into a single polypeptide chain by a flexible linker peptide. After the scFv genes are assembled, they are cloned into a phagemid and expressed at the tip of the M13 phage (or similar filamentous bacteriophage) as fusion proteins with the bacteriophage pIII (gene 3) coat protein. Enriching for phage expressing an antibody of interest is accomplished by panning the recombinant phage displaying a population scFv for binding to a predetermined epitope (e.g., target antigen, receptor).

The linked polynucleotide of a library member provides the basis for replication of the library member after a screening or selection procedure, and also provides the basis for the determination, by nucleotide sequencing, of the identity of the displayed peptide sequence or V_{H} and V_{L} amino acid sequence. The displayed peptide(s) or single-chain antibody (e.g., scFv) and/or its V_{H} and V_{L} regions or their CDRs can be cloned and expressed in a suitable expression system. Often polynucleotides encoding the isolated V_{H} and V_{L} regions will be ligated to polynucleotides encoding constant regions (C_{H} and C_{L}) to form polynucleotides encoding complete antibodies (e.g., chimeric or fully-human), antibody fragments, and the like. Often polynucleotides encoding the isolated CDRs will be grafted into polynucleotides encoding a suitable variable region framework (and optionally constant regions) to form polynucleotides encoding complete antibodies (e.g., humanized or fully-human), antibody fragments, and the like. Antibodies can be used to isolate preparative quantities of the antigen by immunoaffinity chromatography. Various other uses of such antibodies are to diagnose and/or stage disease, and for therapeutic application to treat disease. In the methods of the present invention, the antibodies are used to deliver antigen to dendritic cells in the presence of a dendritic cell maturation factor such that highly efficient antigen presentation occurs in the context of MHC molecules, resulting in long lasting cellular and humoral immunity. In a preferred embodiment, the methods result in induction of robust T cell responses when combined with a dendritic cell maturation factor. Alternatively, when the antigen is targeted to the dendritic cell in the absence of a dendritic cell maturation factor, the methods result in tolerance induction to the antigen.

Various methods have been reported for increasing the combinatorial diversity of a scFv library to broaden the repertoire of binding species. The use of PCR (polymerase chain reaction) has permitted the variable regions to be rapidly cloned either from a specific hybridoma source or as a gene library from non-immunized cells, affording combinatorial diversity in the assortment of V_{H} and V_{L} cassettes which can be combined. Furthermore, the V_{H} and V_{L} cassettes can themselves be diversified, such as by random, pseudorandom, or directed mutagenesis. Typically, V_{H} and V_{L} cassettes are diversified in or near the complementarity-determining regions (CDRs), often the third CDR, CDR3. Enzymatic inverse PCR mutagenesis has been shown to be a simple and reliable method for constructing relatively large libraries of scFv site-directed mutants (Stemmer et al. (1993) Biotechniques 14: 256), as has error-prone PCR and chemical mutagenesis (Deng et al. (1994) J. Biol. Chem. 269: 9533). Riechmann et al. [Biochemistry 32: 8848; (1993)] showed semirational design of an antibody scFv fragment using site-directed randomization by degenerate oligonucleotide PCR and subsequent phage display of the resultant scFv mutants.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

Selection of antibodies specific for DEC-205 is based on binding affinity to DEC-205, preferably human DEC-205 and screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. The standard techniques known in the art for immunoassays are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of DEC-205, one may assay generated hybridomas for a product which binds to a DEC-205 fragment containing such epitope. For selection of an antibody specific to DEC-205 from a particular species of animal, one can select on the basis of positive binding with DEC-205 expressed by or isolated from cells of that species of animal, and the absence of binding to DEC-205 from other species. Binding to DEC-205 may be detected as binding to dendritic cells that express DEC-205.

The foregoing antibodies can be used in methods known in the art relating to the localization and activity of the DEC-205, e.g., for Western blotting, imaging DEC-205 in situ, measuring levels thereof in appropriate physiological samples, etc. The antibodies of the present invention advantageous provide for detecting and enumerating human dendritic cells. Alternatively, such antibodies can be used to isolate human dendritic cells, e.g., by panning. In yet another embodiment, the antibodies of the invention can be used to target molecules to human dendritic cells. It will be recognized that this is a significant advantage, since the prior art antibody of Kraal et al. failed to recognize human DEC-205.

Antibodies that are targeted to DEC-205 and participate in the activity of DEC, e.g., endocytosis, can be generated. Such antibodies can be tested using the assays described supra for identifying ligands. In a specific embodiment, a rabbit polyclonal anti-DEC-205 antibody targets binding of DEC-205, is endocytosed, and is efficiently presented to immunoglobulin-specific T cells.

Targeting Molecules to DEC

The present invention advantageously provides for targeting molecules to DEC-205 for immune modulation, e.g., stimulation of T cell immunity or induction of T cell anergy or tolerance. In particular, an antibody reactive with DEC-205 (or binding portion thereof), as described supra, may be chemically conjugated to a molecule which is to be targeted to DEC-205, for example, an antigen. Alternatively, an antibody that reacts with or binds to DEC-205, is produced recombinantly, such that a genetically modified antibody is generated by splicing the genes for the anti-DEC-205 antibody with the genes encoding the antigen (a chimeric polypeptide). This genetically modified antibody or chimeric polypeptide can be administered to a patient with a dendritic cell maturation factor to elicit efficient antigen presentation and long lasting cellular immunity. In the event that tolerance is desired to the antigen, the antigen/antibody chimeric polypeptide is administered without the additional dendritic cell maturation factor. Another embodiment provides for a further chimeric polypeptide which consists of the anti-DEC-205 antibody amino acid sequence, or a fragment thereof, the amino acid sequence for the antigen and the amino acid sequence for the dendritic cell maturation factor. This molecule would be highly effective at delivery of the antigen to the dendritic cell while concurrently inducing maturation of the dendritic cell such that an immune response is elicited in a manner similar to an active infection, that is, dissemination throughout the lymphatic system for extended periods of time.

Methods for preparation of such chimeric polypeptides, such as the genetically modified anti-DEC-205 antibody contemplated for use in the present invention are known to those skilled in the art.

For example, monoclonal antibodies can be prepared by constructing a recombinant immunoglobulin library, such as a scFv or Fab phage display library and nucleic acid encoding an antibody chain (or portion thereof) can be isolated therefrom. Immunoglobulin light chain and heavy chain first strand cDNAs can be prepared from mRNA derived from lymphocytes of a subject immunized with a protein of interest using primers specific for a constant region of the heavy chain and the constant region of each of the kappa and lambda light chains. Using primers specific for the variable and constant regions, the heavy and light chain cDNAs can then by amplified by PCR. The amplified DNA is then ligated into appropriate vectors for further manipulation in generating a library of display packages. Restriction endonuclease recognition sequences may also be incorporated into the primers to allow for the cloning of the amplified fragment into a vector in a predetermined reading frame for expression on the surface of the display package.

The immunoglobulin library is expressed by a population of display packages, preferably derived from filamentous phage, to form an antibody display library. In addition to commercially available kits for generating phage display libraries (e.g., the Pharmacia Recombinant Phage Antibody System, Catalog No. 27-9400-01; and the Stratagene SurfZAP.TM. Phage Display Kit, Catalog No. 240612), examples of methods and reagents particularly amenable for use in generating antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) Proc. Natl. Acad. Sci. USA 89:3576-3580; Garrad et al. (1991) Bio/Technology 2:1373-1377; Hoogenboom et al. (1991) Nuc Acid Res 19:4133-4137; and Barbas et al. (1991) Proc. Natl. Acad. Sci. USA 88:7978-7982. As generally described in McCafferty et al. Nature (1990) 348:552-554, complete VH and VL domains of an antibody, joined by a flexible (Gly 4-Ser)₃ linker, can be used to produce a single chain antibody expressed on the surface of a display package, such as a filamentous phage.

Once displayed on the surface of a display package (e.g., filamentous phage), the antibody library is screened with a protein of interest, ie. DEC-205, to identify and isolate packages that express an antibody that binds the protein of interest. Display packages expressing antibodies that bind immobilized protein can then be selected. Following screening and identification of a monoclonal antibody (e.g., a monoclonal scFv) specific for the protein of interest, nucleic acid encoding the selected antibody can be recovered from the display package (e.g., from the phage genome) by standard techniques. The nucleic acid so isolated can be further manipulated if desired (e.g., linked to other nucleic acid sequences, for example, to sequences encoding antigens or fragments thereof, or sequences encoding dendritic cell maturation factors) and subcloned into other expression vectors by standard recombinant DNA techniques.

Once isolated, nucleic acid molecules encoding antibody chains, or portions thereof, can be further manipulated using standard recombinant DNA techniques. For example, a single chain antibody gene can also be created by linking a VL coding region to a VH coding region via a nucleotide sequence encoding a flexible linker (e.g., (Gly₄-Ser)₃). Single chain antibodies can be engineered in accordance with the teachings of Bird et al. (1988) Science 242:423-426; Huston et al. (1988) Proc. Natl. Acad. Sci USA 85:5879-5883; Ladner, et al. International Publication Number WO 88/06630; and McCafferty, et al. International Publication No. WO 92/10147. A preferred single chain antibody for use in the invention binds to human DEC-205. A plasmid encoding a scFv antibody to DEC-205 would be prepared using standard molecular biological techniques. Another manipulation that can be performed on isolated antibody genes is to link the antibody gene to a nucleotide sequence encoding an amino acid sequence that directs the antibody homologue to a particular intracellular compartment. A preferred nucleotide sequence to which an antibody gene is linked encodes a signal sequence (also referred to as a leader peptide). Signal sequences are art-recognized amino acid sequences that direct a protein containing the signal sequence at its amino-terminal end to the endoplasmic reticulum (ER). Typically, signal sequences comprise a number of hydrophobic amino acid residues. Alternatively, an antibody homologue can be linked to an amino acid sequence that directs the antibody homologue to a different compartment of the cell. For example, a nuclear localization sequence (NLS) can be linked to the antibody homologue to direct the antibody homologue to the cell nucleus. Nuclear localization sequences are art-recognized targeting sequences. Typically, an NLS is composed of a number of basic amino acid residues.

Following isolation of antibody genes, as described above, and, if desired, further manipulation of the sequences, DNA encoding the antibody can be inserted into an expression vector to facilitate transcription and translation of the antibody coding sequences in a host cell. Within the expression vector, the sequences encoding the antibody are operatively linked to transcriptional and translational control sequences. These control sequences include promoters, enhancers and other expression control elements (e.g., polyadenylation signals). Such regulatory sequences are known to those skilled in the art and are described in Goeddel, Gene Expression Technology: Methods in Enzymology 185, Academic Press, San Diego, Calif. (1990). The expression vector and expression control sequences are chosen to be compatible with the host cell used. Expression vectors can be used to express one antibody chain (e.g., a single chain antibody) or two antibody chains (e.g., a Fab fragment). To express two antibody chains, typically the genes for both chains are inserted into the same expression vector but linked to separate control elements.

Expression of a nucleic acid in mammalian cells is accomplished using a mammalian expression vector. When used in mammalian cells, the expression vector's control functions are often provided by viral material. For example, commonly used promoters are derived from polyoma, Adenovirus 2, cytomegalovirus (CMV) and Simian Virus 40. An example of a suitable mammalian expression vector is pCDNA3 (commercially available from Invitrogen), which drives transcription via the CMV early intermediate promoter/enhancer and contains a neomycin resistance gene as a selective marker. Other examples of mammalian expression vectors include pCDM8 (Seed, B., (1987) Nature 329:840) and pMT2PC (Kaufman et al. (1987), EMBO J. 6:187-195). Alternative to the use of constitutively active viral regulatory sequences, expression of an antibody gene can be controlled by a tissue-specific regulatory element that directs expression of the nucleic acid preferentially in a particular cell type. Tissue-specific regulatory elements are known in the art.

In one embodiment, a recombinant expression vector of the invention is a plasmid vector. Plasmid DNA can be introduced into cells by a variety of techniques either as naked DNA or, more commonly, as DNA complexed with or combined with another substance. Alternatively, in another embodiment, the recombinant expression vector of the invention is a virus, or portion thereof, which allows for expression of a nucleic acid introduced into the viral nucleic acid. For example, replication defective retroviruses, adenoviruses and adeno-associated viruses can be used for recombinant expression of antibody homologue genes. Virally-mediated gene transfer into cells can be accomplished by infecting the target cell with the viral vector.

Non-limiting examples of techniques which can be used to introduce an expression vector encoding an antibody homologue into a host cell include:

Adenovirus-Polylysine DNA Complexes: Naked DNA can be introduced into cells by complexing the DNA to a cation, such as polylysine, which is then coupled to the exterior of an adenovirus virion (e.g., through an antibody bridge, wherein the antibody is specific for the adenovirus molecule and the polylysine is covalently coupled to the antibody) (see Curiel, D. T., et al. (1992) Human Gene Therapy 3:147-154). Entry of the DNA into cells exploits the viral entry function, including natural disruption of endosomes to allow release of the DNA intracellularly. A particularly advantageous feature of this approach is the flexibility in the size and design of heterologous DNA that can be transferred to cells.

Receptor-Mediated DNA Uptake: Naked DNA can also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor facilitates uptake of the DNA by receptor-mediated endocytosis. Receptors to which a DNA-ligand complex have targeted include the transferrin receptor and the asialoglycoprotein receptor. Additionally, a DNA-ligand complex can be linked to adenovirus capsids which naturally disrupt endosomes, thereby promoting release of the DNA material into the cytoplasm and avoiding degradation of the complex by intracellular lysosomes (see for example Curiel et al. (1991) Proc. Natl. Acad. Sci. USA 88:8850; and Cotten, M. et al. (1992) Proc. Natl. Acad. Sci. USA 89:6094-6098; Wagner, E. et al. (1992) Proc. Natl. Acad. Sci. USA 89:6099-6103). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Liposome-Mediated transfection (“lipofection”): Naked DNA can be introduced into cells by mixing the DNA with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel, F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J. Med. Sci. 298:278; and Gould-Fogerite et al. (1989) Gene 84:429438.

Direct Injection: Naked DNA can be introduced into cells by directly injecting the DNA into the cells. For an in vitro culture of cells, DNA can be introduced by microinjection, although this not practical for large numbers of cells. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BiORad).

Retroviral Mediated Gene Transfer: Defective retroviruses are well characterized for use in gene transfer for gene therapy purposes (for a review see Miller, A. D. (1990) Blood 76:271). A recombinant retrovirus can be constructed having a nucleic acid encoding a gene of interest (e.g., an antibody homologue) inserted into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art.

Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:41044115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Adenoviral Mediated Gene Transfer: The genome of an adenovirus can be manipulated such that it encodes and expresses a gene product of interest (e.g., an antibody homologue) but is inactivated in terms of its ability to replicate in a normal lytic viral life cycle. See for example Berkner et al. (i988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to many other gene delivery vectors (Berkner et al. cited supra; Haj-Ahmand and Graham (1986) J. Virol. 57:267). Most replication-defective adenoviral vectors currently in use: are deleted for all or parts of the viral E1 and E3 genes but retain as much as 80% of the adenoviral genetic material.

Adeno-Associated Viral Mediated Gene Transfer: Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of the introduced DNA can be detected, for example, by Northern blotting, RNase protection or reverse transcriptase-polymerase chain reaction (RT-PCR). Expression of the introduced gene product (e.g., the antibody homologue) in the cell can be detected by an appropriate assay for detecting proteins, for example by immunohistochemistry.

As will be appreciated by those skilled in the art, the choice of expression vector system will depend, at least in part, on the host cell targeted for introduction of the nucleic acid.

Antigens for Targeting to Dendritic Cells

The present invention provides for targeting specific antigens derived from microbes, such as viruses or bacteria, as well as tumor cells, to the dendritic cell by coupling these antigens to antibodies specific for particular cell surface structures on dendritic cells. Specifically, the antigens are targeted to the DEC-205 protein on the surface of dendritic cells by way of an antibody specific for DEC-205. The antigen may be coupled to the antibody either by chemical means using standard conjugation techniques known to one skilled in the art, or the antigen may be recombinantly expressed on the same polypeptide chain as the antibody using recombinant techniques known to those skilled in the art. The hybrid or chimeric molecule, ie. the fusion protein, so produced is then taken up by the dendritic cell, and presented in the context of the major hitocompatability complex in a manner that results in highly efficient and persistent antigen presentation, which results in a systemic and long lasting immune response. In particular, the inventors of the present application have shown that the use of such procedures for vaccination purposes results in a robust and long lasting T cell response even with non-replicating antigens.

It has been difficult up to the time of the present invention to be able to achieve such results, especially with T cell responses, wherein it has generally been observed that only live attenuated vaccines could achieve such dramatic T cell responses. It has generally been known that such robust T cell responses could not be achieved with non-replicating vaccines, thus there has always been a need for delivery of the non-replicating vaccine in an adjuvant, in addition for the need for several booster injections. This is not the case using the methods of the present invention. Accordingly, the methods of the present invention provide for a novel strategy for delivery of any microbial or tumor cell antigen to a subject such that a single injection may be sufficient to provide for highly efficient antigen presentation and subsequent immunity. Moreover, the immune response can be directed to either induction of specific T or B cell responses by the concurrent administration of a dendritic cell maturation factor. Alternatively, the sequence of such dendritic cell maturation factor can be incorporated into the hybrid/chimeric molecule, such that upon uptake of the antigen by the dendritic cell by way of the anti-DEC-205 antibody, the dendritic cell maturation factor may also be taken up by the cell and maturation may proceed accordingly. In the event that tolerance to an antigen is desired, the maturation factor would not be administered or would not be incorporated into the chimeric polypeptide (antibody) molecule.

While there are no limitations to the vaccine antigens which may benefit from the methods described herein, a list of several of the antigens which are envisioned for use with the present methods are provided below. The sequences for these antigens are readily available on PubMed using the following key words for search purposes. The particular accession numbers for all of these antigens have been provided below for incorporation into the present application, such that one skilled in the art may practice the methods of the present application using these sequences. Furthermore, certain of these sequences have been provided in the accompanying sequence listing.

Human Immunodeficiency Virus, including the gag, env, pol, tat, rev and nef proteins: Exemplary nucleotide and protein sequences for the full length virus and for the individual proteins noted above are available using the following accession numbers: AF082395; AF414005; AF414001; AF413976; AY227107. Exemplary sequences for the human immunodeficiency virus, and the specific proteins for which a vaccine is contemplated can also be found in SEQ ID NOS: 20, 21, 22, 23, 24, 25 and 26.

Human Papilloma Virus with emphasis on the E6 and E7 molecules:

Accession numbers BC002582, BC009271, NC004500, NC001525, Y18492, Y18491 and E16504. Exemplary sequences can be found in SEQ ID NOS: 27, 28 and 29.

Epstein Barr Virus (EBV):

Accession number BC046112. The sequence is also found in SEQ ID NO: 30.

Malaria circumsporozoite Proteins:

Accession numbers AL034558, AY003872, K02194, and Ml 1145. Exemplary sequences are found in SEQ ID NOS: 31 and 32.

Yersinia pestis:

Accession numbers AF053946, NC003143, NC004088, AF542378, AF528537, AF528536, and AF528535.

Survivin homo sapiens:

Accession numbers AB 154416, BC065497, BC000784, and BC012164. Exemplary sequences can be fouond in SEQ ID NOS: 33 and 34.

Telomerase universal cancer antigens:

Accession numbers BM077067, CF932506, NM198255, NM198254 and NM198253

MAGE family of cancer testis antigens:

Accession numbers AK094541, BC063834, NM177415, NM177404 and NM014599. Exemplary sequences can be found in SEQ ID NOS: 35, 36 and 37.

Furthermore, when an immune response is desired, the method for induction of dendritic cell maturation may be accomplished by several methods described herein. One preferred embodiment is by way of CD40 ligation, such as with an antibody to CD40. The sequence for an anti-CD40 antibody can also be found in PubMed. Several accession numbers are provided below and a few are included in the sequence listing provided in the present application.

Anti-CD40 antibodies:

Accession numbers BD182353, BD182352, BD 182351, BD 182350, BD182349, BD182348, AF487510, AF487509, AF487508, AF487506, AF487505, BD131051, AJ309825, BD131045 and BD131046. Furthermore, several of these sequences may be found in PCT publication No. WO02/088186, in Japanese patent No. JP2002503495 and in Ellmark, P. et al, (2002), Mol. Immunology 39(5-6), 353-360.

Assays for Measuring Immune Responses

The functional outcome of targeting the antigen for which either immunity or tolerance is desired to the dendritic cell via complexing with the anti-DEC-205 antibody can be assessed by suitable assays that monitor induction of cellular or humoral immunity or T cell anergy. These assays are known to one skilled in the art, but may include measurement of cytolytic T cell activity using for example, a chromium release assay. In this assay, one labels a target cell population with radioactive ⁵¹Cr and incubates these labeled target cells with a lymphocyte population obtained from the vaccinated animal at various effector to target ratios. After a suitable incubation time, the supernatants are collected and the amount of chromium released is measured in a gamma counter. The amount of chromium released relates directly to the amount of cell killing by the lymphocytes. This assay is generally used to measure cytolytic T cell responses and the lymphocytes and target cells are matched according to their major histocompatability complex. Alternatively, T cell proliferative assays may be used as an indication of immune reactivity or lack thereof. In addition, in vivo studies can be done to assess the level of protection in a mammal vaccinated against a pathogen or tumor cell using the methods of the present invention. Typical in vivo assays may involve vaccinating an animal with an antigen complexed to the anti-DEC-205 antibody in conjunction with a dendritic cell maturation factor. After waiting for a time sufficient for induction of an antibody or T cell response to occur, generally from about one to two weeks after injection, the animals will be challenged with the antigen, such as either a virus or a tumor cell, and survival of the animals is monitored. A successful vaccination regimen will result in significant survival when compared to the non-vaccinated controls. Serum may also be collected to monitor levels of antibodies generated in response to the vaccine injections.

Immunomodulation. With respect to immunomodulation, the present invention provides for both stimulating T cell-mediated immune responses, particularly for vaccination, and inducing tolerance, particularly with respect to autoimmunity.

Stimulation of T cell immunity can be effected by introducing an antigen, e.g., a weak or poorly immunogenic antigen, conjugated to a DEC-binding moiety (ligand or antibody) into a subject, along with a factor that activates the dendritic cells that initially present antigen to the T cells. Dendritic cell activation can be accomplished by use of an adjuvant, such as an adjuvant as described above, which has the ability to induce a generalized immune response. Alternatively, the “vaccine” of the invention may comprise the antigen conjugated to the DEC-binding moiety and a cytokine or a lymphokine, such as granulocyte-macrophage colony stimulating factor (GM-CSF), or some other CSF. Suitable antigens for use in such a vaccine include bacterial, viral, parasite, and tumor antigens. Moreover, for vaccination purposes, the antigens may be delivered in amounts significantly lower than those amounts generally used for vaccine administration, ie in amounts about 10 to 1000 fold lower than known vaccines for which administration generally necessitates the use of an adjuvant. The antigen, when delivered using the methods of the present invention, may be highly effective and long lasting when delivered only once with or without adjuvant. Moreover, the methods of the present invention provide for highly effective induction of long lasting T cell responses against non-replicating antigens. Thus, the methods of the present invention are contemplated for use with subunit vaccines for a variety of pathogens.

Alternatively, the present invention provides for inducing tolerance. Tolerance is desirable to avoid detrimental immune responses, in particular, autoimmunity and allograft rejection. Presentation of antigen by non-activated dendritic cells, e.g., in the skin and T cell areas of the lymphoid organs, induces T cell anergy, and possibly causes destruction of the responder clone. Thus, in one embodiment, tolerance is induced by administering an antigen modified by conjugation with a DEC-binding moiety under conditions that promote dendritic cell quiescence, e.g., in the absence of an infection, without adjuvant, using pyrogen-free pharmaceutical carriers, and in the absence of additional lymphokines or cytokines.

It is further believed that high level expression of DEC may act as a tolerizing influence. Accordingly, the invention further relates to introducing recombinant dendritic cells, or cell recombinantly modified to express both DEC and MHC Class II, into a subject, along with antigen conjugated to a DEC-binding moiety. Alternatively, the dec gene can be targeted to appropriate cells in vivo, for gene therapy.

In a further embodiment, tolerance can be induced through the clonal deletion mechanism. In particular, antigen conjugated with a DEC-binding moiety can be introduce into a subject, preferably directly into the thymus, either by targeting or physical injection, for processing and presentation by the thymic epithelium and medullary dendritic cells. This processing and presentation step is believed to be involved in the selection process to eliminate autoreactive T cells, i.e., clonal deletion. In a further aspect, the level of expression of DEC may be manipulated, e.g., by introducing additional dec genes into the thymic epithelium and medullary dendritic cells.

Attractive candidates for conjugation with a DEC-ligand to induce tolerance, T cell anergy, or clonal deletion include, but by no means are limited to, allergenic substances, autoantigens such as myelin basic protein, collagen or fragments thereof, DNA, nuclear and nucleolar proteins, mitochondrial proteins, pancreatic β-cell proteins, and the like (see Schwarz, 1993, In Fundamental Immunology, Third Edition, W. E. Paul (Ed.), Raven Press, Ltd.: New York, pp. 1033-1097).

Targeting Vectors for Gene Therapy

In yet another embodiment, the present invention provides ligands for targeting DNA vectors to cells that express DEC, in particular, dendritic cells, epithelial cell of the thymus, small intestine, and lung, and brain capillaries. Accordingly, a DNA vector, and the means for introducing genes into cells has been described above. The viral vectors can be modified to include a ligand for DEC, e.g., by chemically cross-linking a DEC ligand to the virus.

Alternatively, the vector can be introduced in vivo by lipofection, also described above. The use of lipofection to introduce exogenous genes into the specific organs in vivo has certain practical advantages. Molecular targeting of liposomes to specific cells represents one area of benefit. Accordingly, the present invention advantageously provides for targeting a gene for dendritic cells and thymic epithelium by conjugating a DEC-ligand to a liposome vector. Lipids may be chemically coupled to other molecules for the purpose of targeting (see Mackey, et. al., 1988, supra). Targeted antibodies or glycans could be coupled to liposomes chemically.

It is also possible to introduce the vector in vivo as a naked DNA plasmid, also described above, preferably by using a DEC ligand as a vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

Recombinant Vaccine Compositions

The present invention provides genetic vaccines, which include genetic constructs comprising DNA or RNA, which encodes a target protein. As used herein, the term “target protein” refers to a protein capable of eliciting an immune response. The target protein is an immunogenic protein derived from the pathogen or undesirable cell-type such as an infected cell. In accordance with the invention, target proteins are pathogen-associated proteins. The immune response directed against the target protein protects the individual against the specific infection or disease with which the target protein is associated. For example, a genetic vaccine with a DNA or RNA molecule that encodes a pathogen-associated target protein is used to elicit an immune response that will protect the individual from infection by the pathogen. The genetic vaccines of the present invention may encompass not only the nucleic acid sequences of the pathogen for which immunity is desired, but may also comprise the nucleic acid sequences of the antibody to DEC-205. Furthermore, the genetic vaccine may also comprise the nucleic acid from the pathogen, the nucleic acid encoding the DEC-205 antibody or a fragment thereof which when expressed retains its ability to bind to the DEC-205 receptor, and the nucleic acid sequence encoding a dendritic cell maturation factor. In another embodiment, the nucleic acid encoding the antigen from a pathogen may be chemically coupled to the anti-DEC-205 antibody, thus providing more efficient delivery of the nucleic acid to the dendritic cell. In a yet further embodiment, the nucleic acid encoding both the pathogen and the dendritic cell maturation factor may both be chemically coupled to the anti-DEC-205 antibody.

A genetic construct may comprise a nucleotide sequence that encodes a target protein operably linked to regulatory elements needed for gene expression. Accordingly, incorporation of the DNA or RNA molecule into a living cell results in the expression of the DNA or RNA encoding the target protein and thus, production of the target protein.

Following introduction into a cell, a genetic construct comprising a nucleic acid sequence encoding a target protein operably linked to the regulatory elements may be maintained episomally or may be integrated into the cell's chromosomal DNA. DNA may be introduced into cells as a plasmid or as linearized DNA. When introducing DNA into the cell, reagents which promote DNA integration into chromosomes may be added. DNA sequences which are useful to promote integration may also be included in the DNA molecule. Since integration into the chromosomal DNA necessarily requires manipulation of the chromosome, it is preferred to maintain the DNA construct as an episome. This reduces the risk of damaging the cell by splicing into the chromosome and does not adversely alter the effectiveness of the vaccine. Alternatively, RNA may be administered to the cell.

The necessary elements of a genetic construct include a nucleotide sequence that encodes a target protein and the regulatory elements necessary for expression of that sequence in the cells of the vaccinated individual. The regulatory elements are operably linked to the DNA sequence that encodes the target protein to enable expression. The nucleotide sequence that encodes the target protein may be cDNA, genomic DNA, synthesized DNA or a hybrid thereof or an RNA molecule such as mRNA. Accordingly, as used herein, the terms “DNA construct”, “genetic construct” and “nucleotide sequence” may refer to constructs comprising DNA or RNA.

The regulatory elements necessary for gene expression include, but are not limited to, a promoter, an initiation codon, a stop codon, and a polyadenylation signal. It is necessary that these elements be operable in the vaccinated individual. Moreover, it is necessary that these elements be operably linked to the nucleotide sequence that encodes the target protein such that the nucleotide sequence can be expressed in the cells of a vaccinated individual and thus the target protein can be produced.

Initiation codons and stop codons are generally considered to be part of a nucleotide sequence that encodes the target protein. Such sequences may be derived from alternative nucleic acid sources so as to optimize functionality and expression of the target protein in cells of a vaccinated individual. Similarly, promoters and polyadenylation signals used must be functional within the cells of the vaccinated individual.

Examples of promoters useful for practicing this aspect of the present invention, (especially for a genetic vaccine intended for use in humans), include, but are not limited to the Mouse Mammary Tumor Virus (MMTV) promoter, Human Immunodeficiency Virus Long Terminal Repeat (HIV LTR) promoter, Moloney virus promoter, Cytomegalovirus (CMV) promoter, human actin promoter, human myosin promoter, Rous sarcoma virus (RSV) promoter, human hemoglobin promoter, human muscle creatine promoter, and Epstein Barr virus (EBV) promoter.

Examples of polyadenylation signals useful for practicing this aspect of the present invention (especially for a genetic vaccine intended for use in humans), include, but are not limited to SV40 polyadenylation signals and LTR polyadenylation signals.

In addition to the regulatory elements required for DNA expression, other elements may also be included in the DNA molecule. Such additional elements include enhancers. The enhancer may be selected from the group including, but not limited to, the human actin enhancer, human myosin enhancer, CMV enhancer, RSV enhancer, human hemoglobin enhancer, human muscle creatine enhancer, and EBV enhancer.

Genetic constructs may comprise a mammalian origin of replication, the activity of which serves to produce multiple copies of the construct in the cell and thereby, maintain the construct extrachromosomally. Plasmids pCEP4 and pREP4 (Invitrogen, San Diego, Calif.) comprise the EBV origin of replication and nuclear antigen EBNA-1 coding region and achieve high copy episomal replication in the relative absence of integration. Such plasmids may be used in accordance with the invention.

In order to be a functional genetic construct, the regulatory elements must be operably linked to the nucleotide sequence that encodes the target protein. Accordingly, it is necessary for the promoter and polyadenylation signal to be in frame with the coding sequence. In order to maximize protein production, regulatory sequences may be selected which are well suited for gene expression in the vaccinated cells. Moreover, codons may be selected which are most efficiently transcribed in the vaccinated cell.

The immunogenicity of genetic vaccines may also be augmented by rendering them “self-replicating”. RNA vectors encoding an RNA replicase, a polypeptide derived from alphaviruses (such as, e.g., Sindbis virus), are significantly more immunogenic than conventional plasmids. Cells into which a construct comprising an antigen and an RNA replicase has been introduced briefly produce large amounts of antigen before undergoing apoptotic death. Double-stranded RNA (dsRNA) intermediates are thought to trigger both the apoptotic response, which renders the vaccination process self-limiting, and super-activation of dendritic cells, “professional” antigen presenting cells. DNA and RNA-based vaccines and methods of use are described in detail in several publications, including Leitner et al. (1999, Vaccines 18:765-777), Nagashunmugam et al. (1997, AIDS 11: 1433-1444), and Fleeton et al. (2001, J Infect Dis 183:1395-1398) the entire contents of each of which is incorporated herein by reference.

DNA and RNA vaccines may also be rendered more effective by enhancing their uptake into antigen presenting cells, which in turn leads to activation of the cellular immune response, including killer T cells.

In order to test expression, genetic constructs can be tested for expression levels in vitro using tissue culture of cells of the same type as those to be vaccinated. For example, if the genetic vaccine is to be administered into human muscle cells, muscle cells grown in culture such as solid muscle tumor cells of rhabdomyosarcoma may be used as an in vitro model to measure expression level. One of ordinary skill in the art could readily identify a model in vitro system which may be used to measure expression levels of an encoded target protein.

According to the invention, the genetic vaccine may be introduced in vivo into cells of an individual to be immunized or ex vivo into cells of the individual which are re-implanted after incorporation of the genetic vaccine. Either route may be used to introduce genetic material into cells of an individual. Preferred routes of administration include intramuscular, intraperitoneal, intradermal, subcutaneous and intranasal injection. Alternatively, the genetic vaccine may be introduced by various means into cells isolated from an individual. Such means include, for example, transfection, electroporation, and microprojectile bombardment. These methods and other protocols for introducing nucleic acid sequences into cells are known to and routinely practiced by skilled practitioners. After the genetic construct is incorporated into the cells, they are re-implanted into the individual. Prior to re-implantation, the expression levels of a target protein encoded by the genetic vaccine may be assessed. It is contemplated that otherwise non-immunogenic cells that have genetic constructs incorporated therein can be implanted into autologous or heterologous recipients.

The genetic vaccines according to the present invention are formulated according to the mode of administration to be used. One having ordinary skill in the art can readily formulate a genetic vaccine that comprises a genetic construct. In cases where intramuscular injection is the chosen mode of administration, an isotonic formulation is usually used. Generally, additives for isotonicity can include sodium chloride, dextrose, mannitol, sorbitol and lactose. Isotonic solutions such as phosphate buffered saline are preferred. Stabilizers can include gelatin and albumin.

In a preferred embodiment, bupivacaine, a well known and commercially available pharmaceutical compound, is administered prior to or contemporaneously with the genetic construct. Bupivacaine is related chemically and pharmacologically to the aminoacyl local anesthetics. It is a homologue of mepivacaine and related to lidocaine. Bupivacaine renders muscle tissue voltage sensitive to sodium challenge and effects ion concentration within the cells. A complete description of bupivacaine's pharmacological activities can be found in Ritchie, J. M. and N. M. Greene, The Pharmacological Basis of Therapeutics, Eds.: Gilman, A. G. et al, 8th Edition, Chapter 15:3111, which is incorporated herein by reference. Compounds that display a functional similarity to bupivacaine may be useful in the method of the present invention.

Bupivacaine-HCl is chemically designated as 2-piperidinecarboxamide, 1-butyl-N-(2,6-dimethylphenyl)-monohydrochloride, monohydrate and is widely available commercially for pharmaceutical uses from many sources including from Astra Pharmaceutical Products Inc. (Westboro, Mass.) and Sanofi Winthrop Pharmaceuticals (New York, N.Y.), Eastman Kodak (Rochester, N.Y.). About 50 μl to about 2 ml of 0.5% bupivacaine-HCl and 0.1% methylparaben in an isotonic pharmaceutical carrier may be administered to the site where the vaccine is to be administered, preferably, 50 μl to about 1500 μl, more preferably about 1 ml.

The genetic construct may be combined with collagen as an emulsion and delivered intraperitonally. The collagen emulsion provides a means for sustained release of DNA. 50 μl to 2 ml of collagen are used. About 100 μg DNA are combined with 1 ml of collagen in a preferred embodiment using this formulation.

In some embodiments of the invention, the individual is injected with bupivacaine prior to genetic vaccination by intramuscular injection. Bupivacaine may be administered up to, for example, about 24 hours prior to vaccination. Alternatively, bupivacaine may be injected simultaneously or after vaccination.

In some embodiments of the invention, the individual is administered a series of vaccinations to produce a comprehensive immune response. According to this method, at least two and preferably four injections are given over a period of time. The period of time between injections may include from 24 hours apart to two weeks or longer between injections, preferably one week apart. Alternatively, at least two and up to four separate injections may be administered simultaneously at different parts of the body.

In some embodiments of the invention, a complete vaccination includes injection of two or more different inoculants into different tissues. For example, in a vaccine according to the invention, the vaccine comprises two inoculants in which each one comprises genetic material encoding a different viral protein(s). This method of vaccination allows the introduction of a spectrum of viral genes into the individual without the risk of assembling an infectious viral particle. Thus, an immune response against most or all of the immunogenic components of a virus can be invoked in the vaccinated individual. Injection of each inoculant may be performed at different sites, preferably at a distance, to ensure that different genetic constructs are not introduced into the same cell.

While the disclosure herein primarily relates to uses of the methods of the present invention to immunize humans, the methods of the present invention can be applied to veterinary medical uses too. It is within the scope of the present invention to provide methods of immunizing non-human as well as human subjects against pathogens and pathogen protein related disorders and diseases. Accordingly, the present invention relates to genetic immunization of mammals, birds and fish. The methods of the present invention are particularly useful for mammalian species including human, bovine, ovine, porcine, equine, canine and feline species.

While this disclosure generally discusses immunization in the context of prophylactic methods of protection, the term “immunizing” is meant to refer to both prophylactic and therapeutic methods. Thus, a method of immunizing includes both methods of protecting an individual from pathogen challenge, as well as methods for treating an individual suffering from pathogen infection. Accordingly, the present invention may be used as a vaccine for prophylactic protection or in a therapeutic manner; that is, as a reagent for immunotherapeutic methods and preparations.

Therapeutic and Prophylactic Compositions and Their Use

The antibodies and immunogenic compositions of the invention are particularly useful for parenteral administration, i.e., subcutaneously, intramuscularly or intravenously. The compositions for parenteral administration will commonly comprise a solution of an antibody or fragment thereof of the invention or a cocktail thereof dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the antibody or fragment thereof of the invention in such pharmaceutical compositions may vary widely, i.e., from less than about 0.5%, usually at or at least about 1% to as much as 15 or 20% by weight, and will be selected primarily based on fluid volumes, viscosities, etc., according to the particular mode of administration selected. Actual methods for preparing parenterally administrable compositions are well-known or will be apparent to those skilled in the art, and are described in more detail in, e.g., Remington's Pharmaceutical Science, 15th ed., Mack Publishing Company, Easton, Pa.

The antibodies, or fragments thereof, of the invention may be lyophilized for storage and reconstituted in a suitable carrier prior to use.

The pharmaceutical composition of the invention may be administered for prophylactic treatments or for a “therapeutic” purpose. In prophylactic applications, compositions containing the present antibodies or fragments thereof complexed with a predetermined antigen are administered to a subject not already in a disease state but one that may be exposed to a pathogen, in order to enhance the subject's resistance to infection. When provided prophylactically, the compositions are provided before any symptom of infection becomes manifest. The prophylactic administration of the composition serves to prevent or attenuate any subsequent infection. When provided therapeutically, the attenuated or inactivated vaccine is provided upon the detection of a symptom of actual infection. The therapeutic administration of the compound(s) serves to attenuate any actual infection. See, e.g, Berkow, infra, Goodman, infra, Avery, infra and Katzung, infra, which are entirely incorporated herein by reference.

A composition is said to be “pharmacologically acceptable” if its administration can be tolerated by a recipient patient. Such an agent is said to be administered in a “therapeutically effective amount” if the amount administered is physiologically significant. A vaccine or composition of the present invention is physiologically significant if its presence results in a detectable change in the physiology of a recipient patient that enhances at least one primary or secondary humoral or cellular immune response.

The “protection” provided need not be absolute, i.e., the infection need not be totally prevented or eradicated, if there is a statistically significant improvement compared with a control population or set of patients. Protection may be limited to mitigating the severity or rapidity of onset of symptoms of the infection.

According to the present invention, an “effective amount” of a vaccine composition is one that is sufficient to achieve a desired biological effect. It will be recognized by one of skill in the art that the optimal quantity and spacing of individual dosages of an anti-DEC-205 antibody/antigen complex of the invention will be determined by a medical practitioner based on a number of variables including the age, sex, health, and weight of the recipient, kind of concurrent treatment, if any, frequency of treatment, and the nature of the desired outcome. The most preferred dosage will be tailored to the individual subject, as is understood and determinable by one of skill in the art, without undue experimentation. See, e.g., Berkow et al., eds., The Merck Manual, 16th edition, Merck and Co., Rahway, N.J., 1992; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, Mass. (1985); and Katzung, infra, which references and references cited therein, are entirely incorporated herein by reference.

### EXAMPLES

The following examples are presented in order to more fully illustrate the preferred embodiments of the invention. The following examples are put forth so as to provide those of ordinary skill in the art with a complete disclosure and description of how to use the novel constructs described herein, and to provide a suitable means for assaying effectiveness of these constructs and development of pharmaceutical compositions for therapeutic use, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Enhanced Targeting to Dendritic Cells using Anti-DEC-205 Antibody Materials and Methods

Mice

6-8-wk-old females were used in all experiments and were maintained under specific pathogen free conditions. B10.BR, B6.5JL (CD45.1), and B6/MRL (Fas lpr) mice were purchased from The Jackson Laboratory. 3A9 transgenic mice were maintained by crossing with B10.BR mice. To obtain CD45.1 3A9 or 3A9/lprT cells, B6.5JL or B6/MRL mice were crossed extensively with 3A9 mice and tested for CD45.1 and I-A^{k}, by flow cytometry. Fas lpr mutation was tested by PCR. Mice were injected subcutaneously with peptide in CFA and subcutaneously or intravenously with chimeric antibodies. All experiments with mice were performed in accordance with National Institutes of Health guidelines.

Polyclonal antibodies to intact DEC-205—Two New Zealand White rabbits (Hazelton) were injected 6 times with the 205 kDa bands cut from Coomassie-stained, 1.5 mm thick, 4% Duracryl SDS-PAGE gels. Doses ranged from 40-70 Fg of stained protein per animal, per injection (4-6 slices), and were given every 3 weeks, with test bleeds (about 15 ml of serum) taken 2 weeks post-injection. For the first injection, slices were emulsified in Complete Freund's adjuvant (CFA) and injected intradermally into multiple sites on the back. Incomplete Freund's (IFA) was the adjuvant for boosts. Responses were monitored by Western blotting crude thymic membrane extracts with graded doses of serum. Animals were boosted further with the unfractionated eluate from the immunoaffinity column, i.e., soluble protein rather than gel slices. Four boosts, averaging 50 Fg per injection, were given to both rabbits. IgG fractions were prepared by Protein A chromatography.

Polyclonal antibodies to the N-terminal peptide—Peptide N1 from human DEC-205 (SESSGNDPFTIVHENTGKClQPLFD) (SEQ ID NO: 2) was coupled to keyhole limpet hemocyanin (KLH) and ovalbumin (OVA) using maleimide chemistry (Imject, Pierce). An average of about 250 peptides were conjugated to each molecule of KLH, and about 6 peptides per molecule of OVA. The KLH-peptide conjugate was divided into aliquots of 400-500 Fg each, and was injected eight times into two New Zealand White rabbits (200-250 Fg per injection), again emulsifying into CFA for the initial immunization and IFA for boosts. To remove any anti-KLH reactivity from the sera, they were precleared on a KLH-cysteine column. Anti-peptide antibodies were isolated on a peptide-OVA column, where the peptide was coupled to an irrelevant carrier.

Flow Cytometry and Antibodies Used for Staining.

CD4-(L3T4), MHC II-(10-3.6), CD11c-(HL3), CD11c-(HL3), B220-(RA3-6B2), or CD3-(145-2C, CD80(B7-1)-(16-10AI) I-A^{k}-(10-3.6) CD45.1-(A20), I1-2-(JES6-5H4), IFN-γ-(XMG1.2), CD40-(HM40-3-FITC), CD86(B7-2)-(GL1) specific antibodies were from BD PharMingen. Rat IgG-PE (goat anti-rat IgG) specific antibody was from Serotec. 3A9 T cell receptor (1G12)-specific antibody was a gift from Dr. Emil Unanue, Washington University, St. Louis, Mo.

For visualization of rat IgGs on surface of mononuclear cells, lymphoid cells were purified from peripheral LNs 14 h after antibody injection and stained with anti-rat IgG-RPE (goat anti-rat IgG-RPE; Serotec) to visualize surface bound NLDC145 and GL117 antibodies. The cells were then incubated in mouse serum to block nonspecific binding and stained with FITC anti-CD11c (HL3), or -B220 (RA3-6B2), or -CD3 (145-2C).

For intracellular cytokine staining, lymphocytes were stimulated in vitro for 4 h with leukocyte activation cocktail (BD PharMingen) according to the manufacturer's manual. Cells were fixed and permeabilized using cytofix/cytoperm buffer from BD PharMingen.

Immunohistology.

Popliteal LNs were removed from antibody injected mice and 5-μm cryosections (Microm; ZEISS) were prepared. Tissue specimens were fixed in acetone (5 min, room temperature [RT]) air dried, and stained in a moist chamber. The injected antibodies were detected by incubating the sections with streptavidin Cy3 or streptavidin-FITC (Jackson Immunotech). In double labeling experiments, the PE-conjugated antibodies were added for additional 30 nin. Specimens were examined using a fluorescence microscope and confocal optical sections of ˜0.3-μm thickness were generated using deconvolution software (Metamorph).

Constructing and Production of Hybrid Antibodies (Chimeric Polypeptides).

Total RNA was prepared from NLDC-145 and GLI17 (gift of R. J. Hodes, National Institutes of Health, Bethesda, Md.) hybridomas (both rat IgG2a) using Trizol (GIBCO BRL). Full-length Ig cDNAs were produced with 5′-RACE PCR kit (GIBCO BRL) using primers specific for 3′-ends of rat IgG2a and Ig kappa. The V regions were cloned in frame with mouse Ig kappa constant regions and IgG1 constant regions carrying mutations that interfere with FcR binding (Clynes, R. A., (2000), Nat. Med. 6:443446). DNA coding for hen egg lysozyme (HEL) peptide46-61 with spacing residues on both sides was added to the C terminus of the heavy chain using synthetic oligonucleotides. Gene specific primers for cloning of rat IgG2a and Ig kappa: 3′-

| | | |
|---|---|---|
| | 3′-ATAGTTTAGCGGCCGCGATATCTCACTAACACTCATTCCTGTTGAAGCT; | (SEQ ID NO: 7) |
| | 3′-ATAGTTTAGCGGCCGCTCACTAGCTAGCTTTACCAGGAGAGTGGGA- | (SEQ ID NO: 8) |
| | GAGACTCTTCT; | |
| HEL peptide fragment construction: | 5′-CTAGCGACATGGCCAAGAAGGAGACAGTCTGGAGGCTCGAG- | (SEQ ID NO: 9) |
| | GAGTTCGGTAGGTTCACAAACAGGAAC; | |
| | 5′-acagacgtagcacagactatggtattctccagattaacagcaggta | (SEQ ID NO: 10) |
| | ttatgacggtaggacatgataggc; | |
| | 3′-gctgtaccggttcttcctctgtcagacctccgagctcctcaa- | (SEQ ID NO: 11) |
| | gccatccaagtgtttgtccttgtgtctg; | |
| | 3′-CCATCGTGTCTGATACCATAAGAGGTCTAATTGTCGTCCATAATAC | (SEQ ID NO: 12) |
| | TGCCATCCTGTACTATCCGCCGG. | |

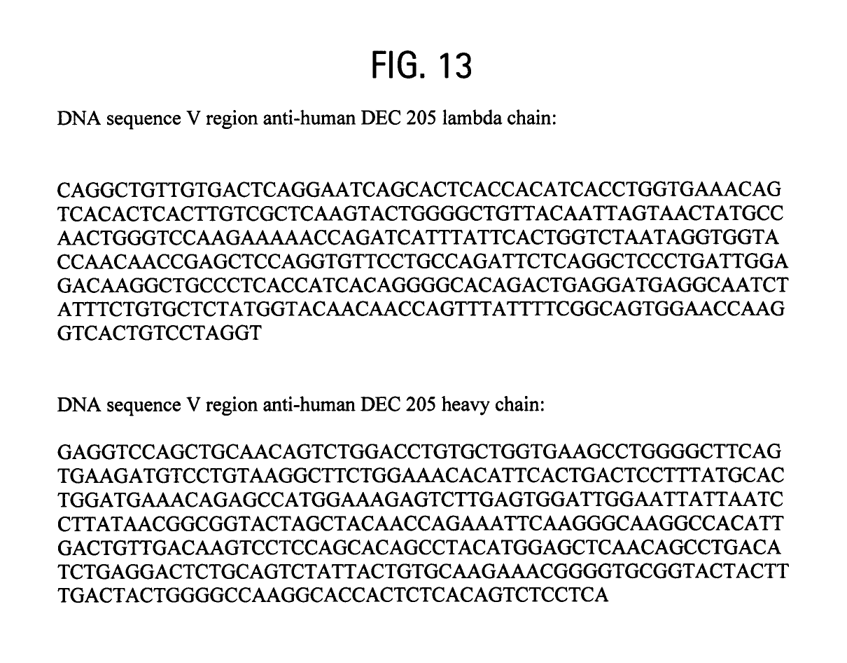

The anti-DEC-205 V region DNA sequences for the lambda and the heavy chains of the antibody can be found in FIG. 13, as SEQ ID NOS: 13 and 14. The anti-human CD40 antibody sequence can be found as SEQ ID NOs: 17, 18 and 19.

Hybrid antibodies were transiently expressed in 293 cells after transfection using calcium-phosphate. Cells were grown in serum-free DMEM supplemented with Nutridoma SP (Boehringer). Antibodies were purified on Protein G columns (Amersham Pharmacia Biotech). The concentrations of purified antibodies were determined by ELISA using goat anti-mouse IgG1 (Jackson Imunotech).

Cell Culture and Proliferation Assays.

Pooled axillary, brachial, inguinal, and popliteal LNs were dissociated in 5% FCS RPMI and incubated in presence of collagenase (Boehringer) and EDTA as described (21). For antigen presentation CD19⁺ and CD11c⁺ cells were purified using microbeads coupled to anti-mouse CD11c or CD19 IgG (Miltenyi Biotec) and irradiated with 1,500 rad. CD4 T cells were purified by depletion using rat antibodies supernatants specific for mouse: CD8 (TIB 211), B220 (RA3-6B2), MHC II (M5/114, TIB 120), F4/80 (F4/80), and magnetic beads coupled to anti-rat IgG (Dynal). In antigen loading experiments the isolated presenting cells from each experimental group were cultured in 96-well plates with 2×10⁵ purified 3A9 CD4⁺ T cells. Cultures were maintained for 48 h with [³H]thymidine (1 μCi) added for the last 6 h. The results were calculated as a ratio of prQliferation in experimental groups to a PBS control group. The proliferation in PBS controls ranged from 500 to 2,000 cpm.

For T cell proliferation assays in adoptive transfer recipients, 9×10⁴ of the same irradiated CD11c⁺ cells isolated from spleens of wild-type B10.BR mice were cultured in 96-well plates with 3×10⁵ T cells from each experimental group. Synthetic HEL peptide, at final concentration of 100 μg/m], was added to half of the cultures. Cultures were maintained for 24 h with [³H]thymidine (1 μCi/ml) added for the last 6 h. Responseto HEL peptide was determined by subtracting background (no HEL peptide added) proliferation from proliferation in the presence of HEL peptide. Proliferation index was calculated as the ratio of the response to HEL peptide in a given experimental group to the response to HEL of T cells from a PBS-injected control. Proliferation in PBS groups ranged from 4,000-8,000 cpm in the presence of peptide and the response to HEL peptide in these PBS controls was 1,000-3,000 counts above the background. Synthetic HEL 46-61 peptide was provided by the Howard Hughes Medical Institute Keck Biotechnology Resource Center.

Adoptive Transfer.

CD4 cells from 3A9 mice were enriched by depletion as described above, washed 3× with PBS, and 5×10⁶ cells injected intravenously per mouse. Alternatively, before depletion total cells were labeled with 2 μM 5-(6)-carboxyfluorescein diacetate succinimidyl diester (CFSE) in 5% FCS RPMI (Molecular Probes) at 37° C. for 20 min and washed twice.

Results

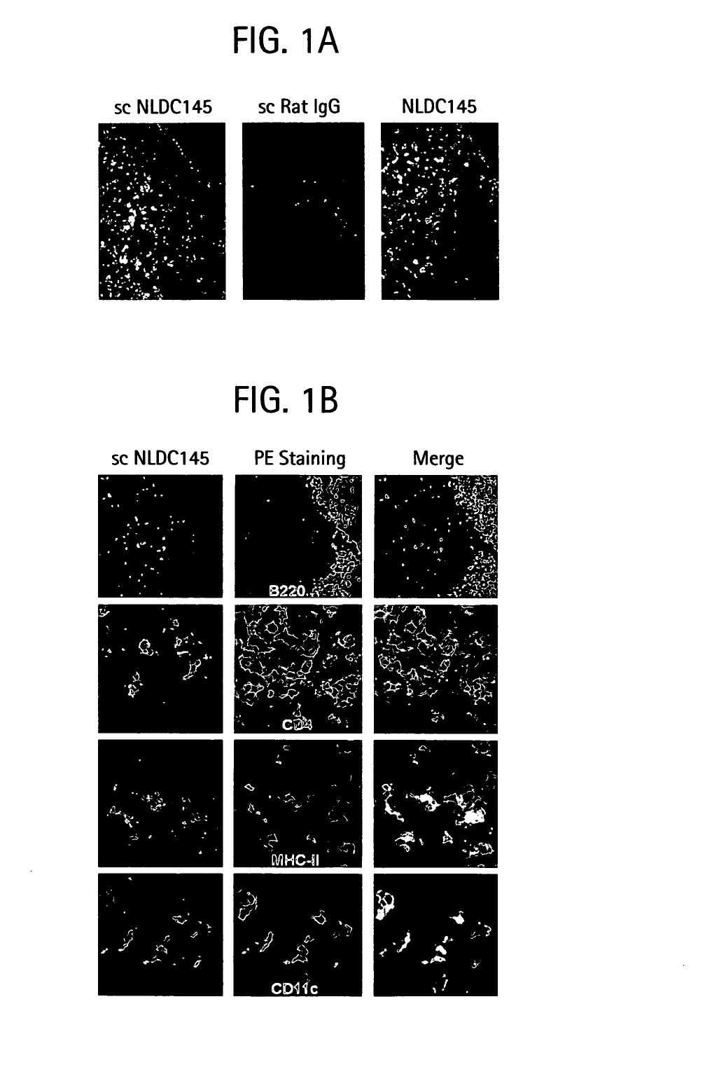

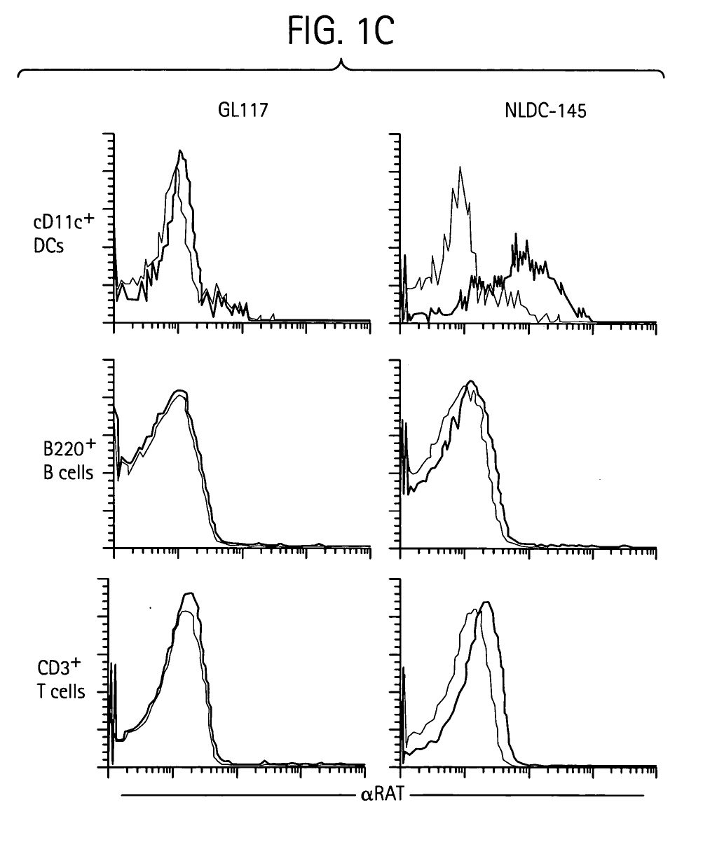

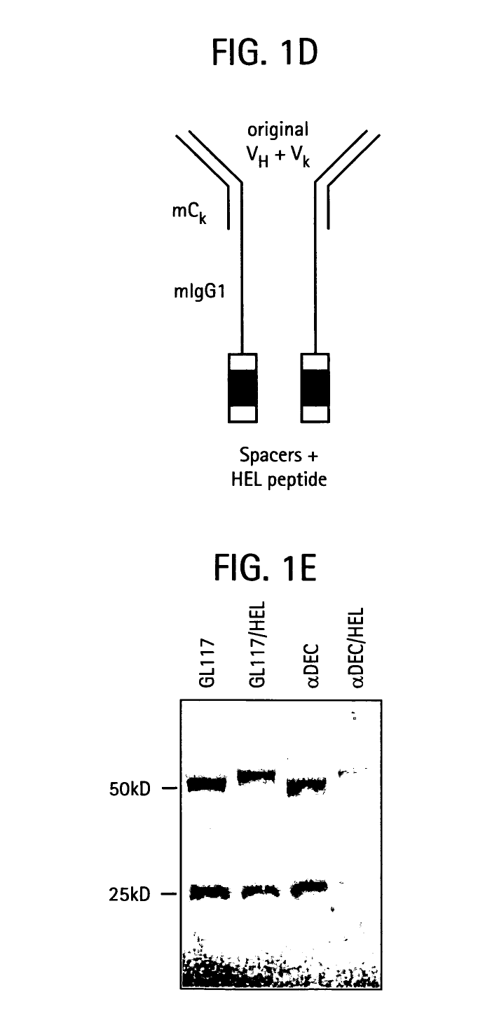

To determine whether the NLDC145 antibody targets DCs in vivo, mice were injected subcutaneously with purified NLDC145 or GL117, a non-specific isotype-matched rat monoclonal antibody control, and visualized the injected antibody in tissue sections. Popliteal lymph nodes (LNs) were removed from antibody-injected mice and 5 μm cryosections (Microm, Zeiss, Germany) were prepared. Tissue specimens were fixed in acetone (5 min, RT) air dried and stained in a moist chamber. The injected antibodies were detected by incubating the sections with streptavidin Cy3 or streptavidin-FITC (Jackson Immunotech). In double labeling experiments, the PE conjugated antibodies were added for additional 30 min. Specimens were examined using a fluorescence microscope and confocal optical sections of approx. 0.3 μm thickness were generated using deconvolution software (Metamorph). Twenty-four hours after injection, NLDC145 was found localized to scattered large dendritic profiles in the T cell areas of lymph nodes and spleen while uptake of control GL117 was undetectable (FIG. 1A left and middle). This pattern was similar to the pattern found when the antibody was applied to sections directly (FIG. 1A right). The NLDC145-targeted cells were negative for B220 and CD4, markers for B cells and T cells respectively, but positive for characteristic DC markers including MHC II and CD11c (FIG. 1B). Thus, subcutaneously injected NLDC145 targets specifically to CD11c⁺ MHC II⁺ DCs in lymphoid tissues in vivo. To further characterize the lymphoid cells that were targeted by NLDC145 in vivo, lymphoid cell suspensions from antibody injected mice were stained with anti-rat Ig and the cells were examined by multiparameter flow cytometry (FIG. 1C). High levels of injected NLDC145 were found on the surface of most CD11c+DCs but not on the surface of B220³⁰ B cells or CD3⁺ T cells (FIG. 1C). This shows that when NLDC145 is injected into mice it binds efficiently and directly to DCs but not to other lymphoid cells. To deliver antigens to DCs in vivo, fusion proteins were produced with amino acids 46-61 of hen egg lysozyme (HEL) added to the carboxyl terminus of cloned NLDC145 (αDEC/HEL) and GL117 (GL117/HEL) control antibody (FIG. 1D). Total RNA was prepared from NLDC-145 (C. Kurts, H. Kosaka, F. R. Carbone, J. F. Miller, W. R. Heath, J Exp Med 186, 239-45. (1997)) and GLII7 (gift of R. J. Hodes) hybridomas (both rat IgG2a) using Trizol (GibcoBRL). Full-length Ig cDNAs were produced with 5′-RACE PCR kit (GibcoBRL) using primers specific for 3′-ends of rat IgG2a and Ig kappa. The V regions were cloned in frame with mouse Ig kappa constant regions and IgG1 constant regions carrying mutations that interfere with FcR binding (K. Mahnke, et al., J Cell Biol 151, 673-84 (2000)). DNA coding for HEL peptide 46-61 with spacing residues on both sides was added to the C terminus of the heavy chain using synthetic oligonucleotides. Gene specific primers for cloning of rat IgG2a and Ig kappa:

| | |
|---|---|
| 5′ATAGTTTAGCGGCCCGCGATATCTCACTAACACTCATTCCTGTTGAAGCT; | (SEQ ID NO:7) |
| 3′ATAGTTTAGCGGCCGCTCACTAGCTAGCTTTACCAGGAGAGTGGGAG-AGACTCTTCT. | (SEQ ID NO:8) |

HEL peptide fragment construction:

| | |
|---|---|
| 5′CTAGCGACATGGCCAAGAAGGAGACAGTCTGGAGGCTCGAGGAGTTCGGT | (SEQ ID NO:9) |
| AGGTTCACAAACAGGAAC | |
| 5′ACAGACGGTAGCACAGACTATGGTATTCTCCAGATTAACAGCAGGTATTAT | (SEQ ID NO:10) |
| GACGGTAGGACATGATAGGC | |
| 3′GCTGTACCGGTTCTTCCTCTGTCAGACCTCCGAGCTCCTCAAGCCATCCAAG | (SEQ ID NO:11) |
| TGTTTGTCCTTGTGTCTG | |
| 3′CCATCGTGTCTGATACCATAAGAGGTCTAATTGTCG-TCCATAATACTGCCAT | (SEQ ID NO:12) |
| CCTGTACTATCCGCCGG. | |

To minimize antibody binding to Fc (FcR) receptors and further ensure the specificity of antigen targeting, the rat IgG2a constant regions of the original antibodies were replaced with mouse IgG1 constant regions that carry point mutations interfering with FcR binding (R. A. Clynes, T. L. Towers, L. G. Presta, J. V. Ravetch, Nat Med 6, 443-6 (2000)). The hybrid antibodies and control Igs without the terminal HEL (αDEC and GL117) were produced by transient transfection in 293 cells (FIG. 1E). Hybrid antibodies were transiently expressed in 293 cells after transfection using calcium phosphate. Cells were grown in serum free DMEM supplemented with Nutridoma SP (Boehringer). Antibodies were purified on Protein G columns (Pharmacia). The concentrations of purified antibodies were determined by ELISA using goat anti-mouse IgG1 (Jackson Immunotech).

Detailed description of FIG. 1: FIG. 1.NLDC-145 targets DCs in vivo. (A) Biotinylated NLDC-145 (scNLDC145 left) or rat IgG (scRatIgG middle) was injected into the hind footpads (50 μg/footpad) and inguinal lymph nodes harvested 24 hours later. Sections were stained with Streptavidin Cy3. Control sections from uninjected mice were stained using biotinylated NLDC145 and streptavidin Cy3 (NLDC145 right). (B) Two color immunofluorescence. Mice were injected with biotinylated NLDC145 as in (A) Sections were stained with streptavidin FITC (green) and PE-labeled antibodies (red) to B220 clone (RA3-6B2), CD4 (L3T4), MHC II (10-3.6), or CD11c clone (HL3) (all from PharMingen) as indicated. Specimens were analyzed by deconvolution microscopy. Double labeling is indicated by the yellow color. (C) FACS analysis of lymphoid cells after injection with NLDC145 and control GL117 antibody. B10.BR mice were injected subcutaneously in the footpads with 10 μg of NLDC145, or GL117 antibodies or PBS. Lymphoid cells were purified from peripheral lymph nodes 14 hours after antibody injection and stained with anti-rat IgG-RPE (Goat Anti-Rat IgG-RPE Serotec, UK) to visualize surface bound NLDC145 and GL117 antibodies. The cells were then incubated in mouse serum to block non-specific binding and stained with FITC anti-CD11c (HL3), or -B220 (RA3-6B2), or -CD3 (145-2C11); all antibodies were from PharMingen. Histograms show staining with anti-rat IgG on gated populations of CD 11c⁺ DCs, B220⁺ B cells and CD3⁺ T cells. (D) Diagrammatic representation of hybrid antibodies. Heavy and light chain constant regions of GL117 and NLDC145 monoclonal antibodies were replaced with mouse Ig kappa (mCk) and IgG1 constant (mIgG1) regions containing mutations that interfere with FcR binding. Sequences encoding the 46-61 HEL peptide with flanking spacer residues were added to the carboxyl ends of the heavy chains. (E) Hybrid antibodies. GL117, GL117/HEL, αDEC and αDEC/HEL antibodies analyzed by PAGE under reducing conditions, molecular weights in kD are indicated.

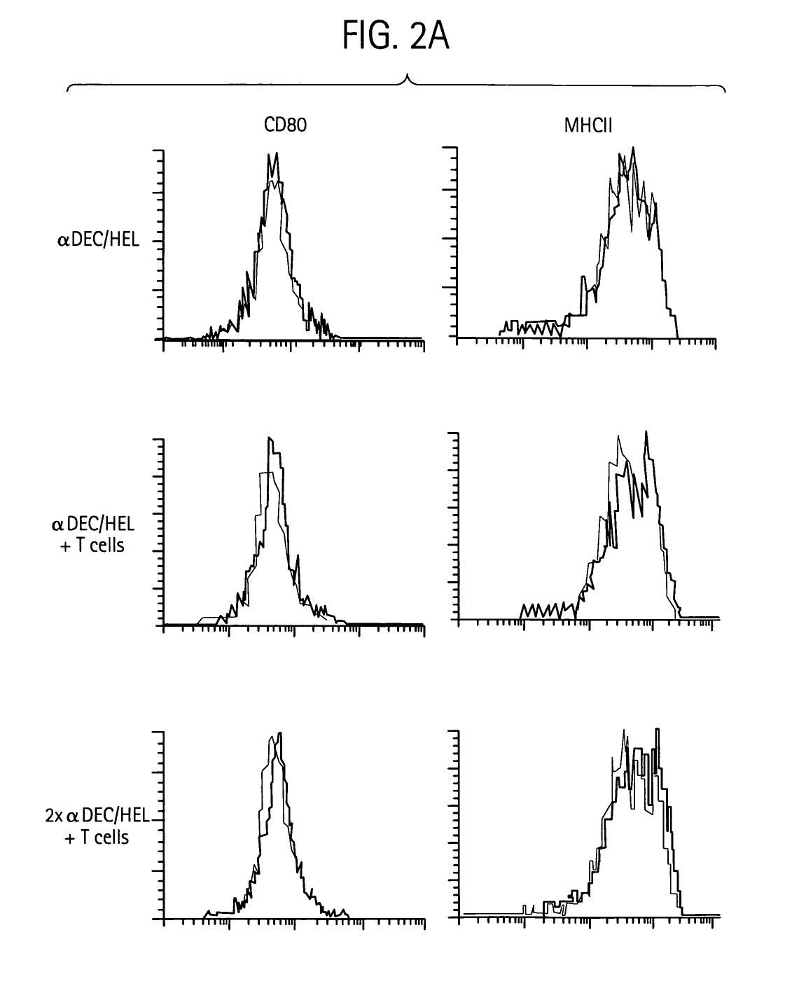

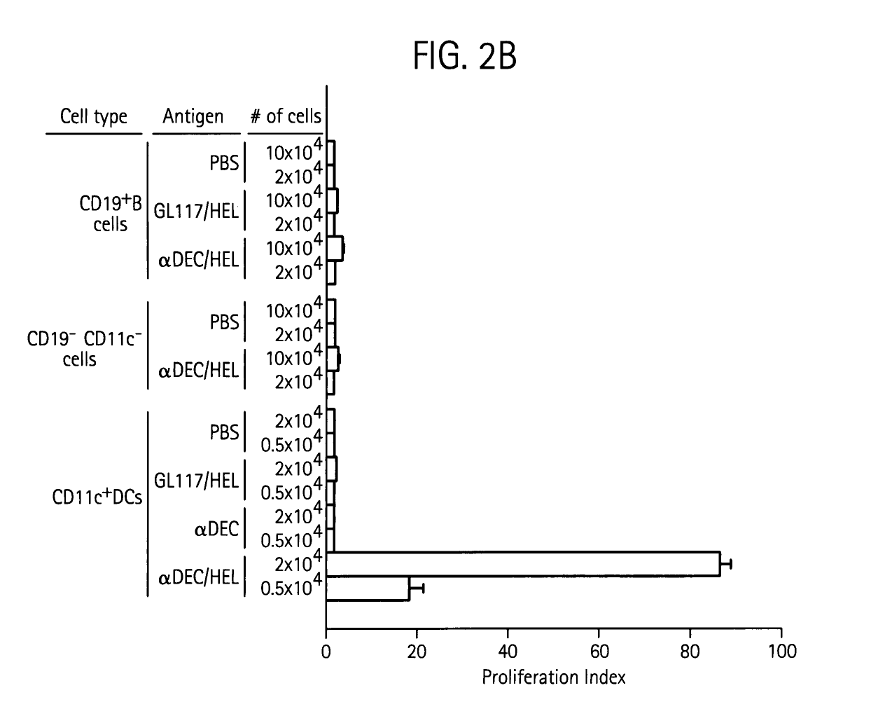

To determine whether antigens delivered by αDEC/HEL were processed by DCs in vivo, we injected mice with the hybrid antibodies and controls and tested CD11c.sup.+DCs, CD19+B cells and CD11c-CD19-mononuclear cells for their capacity to present HEL peptide to nave HEL-specific T cells from 3A9 TCR transgenic mice (W. Y. Ho, M. P. Cooke, C. C. Goodnow, M. M. Davis, J Exp Med 179, 1539-49 (1994)). Six to 8 week old females were used in all experiments and were maintained under specific pathogen free conditions. B10.BR, B6.5JL (CD45.1) and B6/MRL (Fas lpr) mice were purchased from Jackson Laboratory. 3A9 transgenic mice were maintained by crossing with B10.BR mice. To obtain CD45.1 3A9 or 3A9/lpr T cells B6.5JL or B6/MRL mice were crossed extensively with 3A9 mice and tested for CD45.1 and I-Ak, by flow cytometry. Fas lpr mutation was tested by PCR. Mice were injected subcutaneously (s.c.) with peptide in CFA and s.c.or intravenously with chimeric antibodies. All experiments with mice were performed in accordance with NIH guidelines. DCs isolated from antibody-injected mice expressed levels of CD80 and MHC II similar to those found on PBS controls and thus showed no signs of increased maturation, in contrast to what occurs when DCs are stimulated with microbial products like bacterial lipopolysaccharide (LPS) and CpG deoxyoligonucleotides (T. De Smedt, et al., Journal of Experimental Medicine 184, 1413-24 (1996); T. Sparwasser, R. M. Vabulas, B. Villmow, G. B. Lipford, H. Wagner, European Journal of Immunology 30, 3591-7 (2000)) (FIG. 2A). Nevertheless DCs from mice injected with αDEC/HEL induced strong T cell proliferative responses, whereas DCs isolated from PBS-injected mice or mice injected with the control antibodies had no effect (FIG. 2B). Pooled axillary, brachial, inguinal and popliteal lymph nodes were dissociated in 5% FCS RPMI and incubated in presence of collagenase (Boehringer) and EDTA as described (Hochrein et al., 2001, Differential production of IL-12, IFN-alpha, and IFN-gamma by mouse dendritic cell subsets. J Immunol 166:5448-55). For antigen presentation CD19+ and CD11c+ were purified using microbeads coupled to anti-mouse CD11c or CD19 IgG (Miltenyi) and irradiated with 1500 R. CD4 T cells were purified by depletion using rat antibodies supernatants specific for mouse: CD8 (TIB 211), B220 (RA3-6B2), MHC II (M5/114, TIB 120), F4/80 (F4/80,) and magnetic beads coupled to anti-rat IgG (Dynal). In antigen loading experiments the isolated presenting cells from each experimental group were cultured in 96-well plates with 2×10⁵ purified 3A9 CD4⁺ T cells. Cultures were maintained for 48 h with ³H-thymidine (1 microCi) added for the last 6 h. The results were calculated as a ratio of proliferation in experimental groups to a PBS control group. The proliferation in PBS controls ranged from 500 to 2000 cpm.

For T cell proliferation assays in adoptive transfer recipients, 9×10⁴ of the same irradiated CD11c+ cells isolated from spleens of WT B10.BR mice were cultured in 96-well plates with 3×10⁵ T cells from each experimental group. Synthetic HEL peptide, at final concentration of 100 microgram/ml, was added to half of the cultures. Cultures were maintained for 24 h with ³H-thymidine (1 microCi/ml) added for the last 6 h.

Response to HEL peptide was determined by subtracting background (no HEL peptide added) proliferation from proliferation in the presence of HEL peptide. Proliferation index was calculated as the ratio of the response to HEL peptide in a given experimental group to the response to HEL of T cells from a PBS injected control. Proliferation in PBS groups ranged from 4000-8000 cpm in the presence of peptide and the response to HEL peptide in these PBS controls was 1000-3000 counts above the background. Synthetic HEL 46-61 peptide was provided by the HHMI Keck Biotechnology Resource Center. DC isolated 3 days after αDEC/HEL injection showed reduced antigen-presenting activity (data not shown). In contrast to DCs, B cells and bulk CD11c⁻ CD19⁻ mononuclear cells purified from the same mice showed little antigen presenting activity (FIG. 2B). We conclude that antigens can be selectively and efficiently delivered to DC by αDEC/HEL in vivo, and the targeted DCs successfully process and load the peptides onto MHC II.

Detailed description of FIG. 2: DCs process and present antigen delivered by hybrid antibodies. (A) MHC II and CD80 expression on DCs is not altered by multiple injections of αDEC/HEL and 3A9 T cells. B10.BR mice transferred with 3A9 T cells and controls were injected subcutaneously in the footpads with 0.2 μg αDEC/HEL or PBS either at 8 days (αDEC/HEL) or at 1 and 8 days (αDEC/HELX2) after transfer (similar results were obtained by intravenous injection of chimeric antibodies—data not shown). Twenty-four hours after the last αDEC/HEL injection, DCs were purified from peripheral lymph nodes and analyzed by flow cytometry for expression of CD80 and MHC II (anti-CD80(B7-1)(16-10A1)) and anti-I-A^{k} (10-3.6), respectively; PharMingen). Dotted lines in histograms indicate PBS control. (B) αDEC/HEL delivers HEL peptide to DCs in vivo. B10.BR mice were injected subcutaneously into footpads with 0.3 μg of αDEC/HEL or GL117/HEL or αDEC or PBS as indicated. CD11c⁺, CD19⁺ and CD11c⁻ CD19-cells were isolated from draining lymph nodes 24 hours after antibody injection and assayed for antigen processing and presentation to purified 3A9 T cells in vitro. T cell proliferation was measured by ³H-thymidine incorporation and is expressed as a proliferation index relative to PBS controls. The results are means of triplicate cultures from one of four similar experiments.

Adoptive transfer experiments with HEL-specific transgenic T cells were performed to follow the response of these T cells to otherwise unmanipulated, antigen-targeted DC in vivo. CD4⁺ 3A9 T cells were transferred into B10.BR recipients. CD4 cells from 3A9 mice were enriched by depletion, washed 3.times. with PBS and 5×10⁶ cells injected intravenously per mouse. Alternatively, before depletion total cells were labeled with 2 μM CFSE in 5% FCS RPMI (Molecular Probes) at 37° C. for 20 min and washed twice and 24 h later hybrid antibodies were injected subcutaneously. To measure T cell responses, CD4⁺ cells were isolated from the draining lymph nodes of the injected mice and cultured in vitro in the presence or absence of added HEL peptide. Pooled axillary, brachial, inguinal and popliteal lymph nodes were dissociated in 5% FCS RPMI and incubated in presence of collagenase (Boehringer) and EDTA. For antigen presentation CD 19+ and CD11c+ were purified using microbeads coupled to anti-mouse CD11c or CD19 IgG (Miltenyi) and irradiated with 1500 R. CD4 T cells were purified by depletion using rat antibodies supernatants specific for mouse: CD8 (TIB 211), B220 (RA3-6B2), MHC II (M5/114, TIB 120), F4/80 (F4/80,) and magnetic beads coupled to anti-rat IgG (Dynal). In antigen loading experiments the isolated presenting cells from each experimental group were cultured in 96-well plates with 2×10⁵ purified 3A9 CD4⁺ T cells. Cultures were maintained for 48 h with ³H-thymidine (1 microCi) added for the last 6 h. The results were calculated as a ratio of proliferation in experimental groups to a PBS control group. The proliferation in PBS controls ranged from 500 to 2000 cpm.

For T cell proliferation assays in adoptive transfer recipients, 9×10⁴ of the same irradiated CD11c+ cells isolated from spleens of WT B10.BR mice were cultured in 96-well plates with 3×10⁵ T cells from each experimental group. Synthetic HEL peptide, at final concentration of 100 microg/ml, was added to half of the cultures. Cultures were maintained for 24 h with 3H-thymidine (1 microCi/ml) added for the last 6 h. Response to HEL peptide was determined by subtracting background (no HEL peptide added) proliferation from proliferation in the presence of HEL peptide. Proliferation index was calculated as the ratio of the response to HEL peptide in a given experimental group to the response to HEL of T cells from a PBS injected control. Proliferation in PBS groups ranged from 4000-8000 cpm in the presence of peptide and the response to HEL peptide in these PBS controls was 1000-3000 counts above the background. Synthetic HEL 46-61 peptide was provided by the HHMI Keck Biotechnology Resource Center. T cell responses were measured by ³H-thymidine incorporation and are shown as proliferation indices normalized to the PBS control (this index facilitates comparison between experiments, see (31)). In addition to αDEC/HEL, GL117/HEL, αDEC and GL117 antibodies, we included 100 μg of HEL peptide in complete Freund's adjuvant (CFA) as a positive control.

As described in previous reports (E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998)), CD4⁺ T cells isolated 2 days after challenge with 100 μg of HEL peptide in CFA showed strong proliferative responses to antigen when compared with PBS controls (FIG. 3A). Similar responses were obtained from mice injected with as little as 0.2 μg of αDEC/HEL (i.e., .about.4 ng peptide per mouse) but not from mice injected with up to 1 μg of αDEC, GL117 or GL117/HEL controls (FIG. 3A and not shown). We conclude that antigen delivered to DCs in vivo by αDEC/HEL efficiently induces activation of specific T cells.

To determine whether antigen delivered to DCs in vivo induces persistent T cell activation, we measured T cell responses to antigen 7 days after the administration of α DEC/HEL. CD4 T cells continued to show heightened responses to antigen when purified from LNs 7 days after injection with 100 μg of HEL peptide in CFA (E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998)) (FIG. 3B). In contrast, T cells isolated from mice 7 days after injection with αDEC/HEL were no longer activated when compared to PBS controls (FIG. 3B). Thus, T cell activation by antigen delivered to DCs by αDEC/HEL in vivo is transient, readily detected at 2 but not 7 days. This transient activation resembles the CD4 T cell response to large doses of peptide in the absence of adjuvant, or the response to self antigens presented by bone marrow derived antigen presenting cells in the periphery (C. Kurts, H. Kosaka, F. R. Carbone, J. F. Miller, W. R. Heath, J Exp Med 186, 23945, (1997); D. J. Morgan, H. T. Kreuwel, L. A. Sherman, J Immunol 163, 723-7. (1999), E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998); P. Aichele, K. Brduscha-Riem, R. M. Zinkernagel, H. Hengartner, H. Pircher, J Exp Med 182, 261-6 (1995)). To determine whether the absence of persistent T cell activation in mice injected with αDEC/HEL is due to clearance of the injected antigen, multiple doses of αDEC/HEL were administered. Repeated injection of a DEC/HEL at 3-day intervals failed to induce prolonged T cell activation (FIG. 3C). In addition, after 7 or 20 days, T cells initially activated by αDEC/HEL could not be re-activated when the mice were challenged with 100 μg of HEL peptide in CFA (FIG. 3D). Thus, the transient nature of the T cell response in mice injected with α DEC/HEL is not due to a lack of antigen, and T cells initially activated by DCs under physiologic conditions are unresponsive to subsequent challenge with antigen even in the presence of strong adjuvants.

Absence of persistent T cell responses could be due to DC deletion, T cell deletion, or induction of T cell anergy. To assess DC function in mice receiving multiple doses of a DEC/HEL, we isolated DCs from these mice and monitored presentation to 3A9 T cells in vitro (FIG. 3E) (see above methods). DCs from mice injected with two doses of antibody showed the same T cell stimulatory activity as DCs isolated from mice receiving a single injection of α DEC/HEL (FIG. 3E). In addition, the transfer of antigen specific T cells into a DEC/HEL recipients did not alter the ability of the isolated DCs to stimulate 3A9 T cells in vitro. Thus, the transient nature of the T cell response to DC-targeted-antigens in vivo is not the result of a lack of antigen-bearing DCs.

Detailed description of FIG. 3: In vivo activation of CD4+ T cells by a DEC/HEL. In all experiments, 3A9 T cells were transferred into B 10.BR mice, and the recipients were injected subcutaneously in the footpads with antibodies in PBS or 100 μg of HEL peptide in CFA 24 hours after T cell transfer as indicated. T cell proliferation was measured by ³H-thymidine incorporation and is expressed as a proliferation index relative to PBS controls. (A) T cells are efficiently activated by antigen delivered by a DEC/HEL. 48 h after challenge with antigen in the indicated doses, CD4 T cells were isolated from peripheral lymph nodes and cultured in vitro with irradiated B10.BR CD11c+ cells in the presence or absence of HEL peptide. (B) CD4⁺ T cells are only transiently activated by antigen (a DEC/HEL 0.2 μg) delivered to DCs in vivo. CD4+ cells were purified from peripheral lymph nodes 2 or 7 days after challenge with antigen and cultured with irradiated CD11c+ cells in the presence or absence of HEL peptide. (C) Failure to induce persistent T cell activation with multiple injections of a DEC/HEL. 3A9 cells were transferred into B10.BR mice and recipients were injected with a DEC/HEL (0.2 μg/mouse) once (on day 9 or 2 before analysis) or multiple times (days 9, 6 and 2 before analysis). Assay for T cell activation was as above. (D) T cells initially activated by a DEC/HEL show diminished response to re-challenge with HEL peptide in CFA. Recipients were initially injected with either a DEC/HEL (0.2 μg), GL117/HEL (0.2 μg) or PBS and re-challenged 7 or 20 days later with 100 μg of HEL peptide in CFA or with PBS. CD4+ cells were purified from peripheral lymph nodes 2 days after the re-challenge and cultured with irradiated CD11c+ cells in the presence or absence of HEL peptide. Assay for T cell activation was as above. (E) Antigen loading of DCs with a DEC/HEL. B10.BR mice+/−transferred 3A9 T cells, were injected subcutaneously with 0.2 μg a DEC/HEL or PBS either at 8 days (α DEC/HEL) or at 1 and 8 days α.DEC/HELX2) after transfer. Antigen loading was measured 1 day after the last dose of α DEC/HEL by purifying CD11c+ DCs from peripheral lymph nodes and culturing with purified 3A9 T cells. The results are means of triplicate cultures from one of three similar experiments.

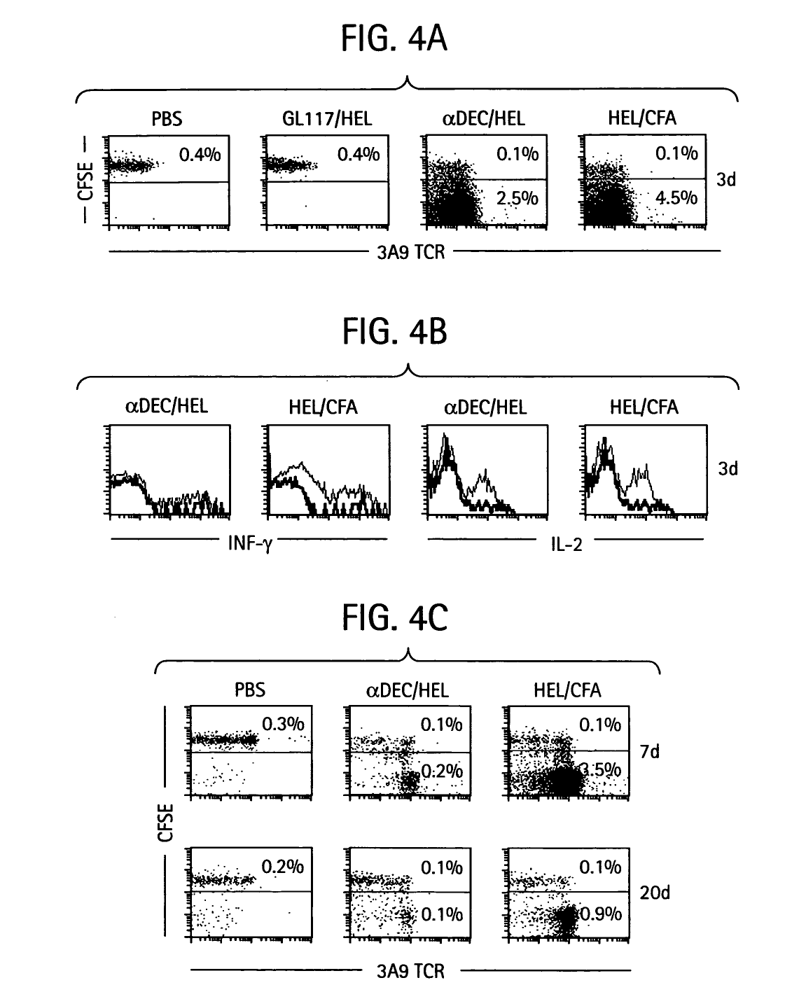

To examine the fate of 3A9 T cells after exposure to antigen presented by DCs in vivo, we performed adoptive transfer experiments with CD45.1⁺3A9 T cells labeled with 5-(6)-carboxyfluorescein diacetate succinimidyl diester (CFSE), a reporter dye for cell division. As previously described, T cells challenged with peptide in CFA divide, upregulate CD69 but not CD25 and produce IL-2 and IFN.γ but not IL4 or IL-10. These cells are therefore considered to be Th1 polarized (FIGS. 4A, B and not shown) (E. R. Kearney, K. A. Pape, D. Y. Loh, M. K. Jenkins, Immunity 1, 327-39 (1994); L. Van Parijs, D. A. Peterson, A. K. Abbas, Immunity 8, 265-74 (1998)). A burst of cell division and increase of CD69 but not CD25 expression was also seen after injection with 0.2 μg α DEC/HEL but not with GL117/HEL. Only clonotype positive CD4 cells showed these effects (FIGS. 4A, C and not shown). However, 3A9 cells activated by antigen presented on a DEC/HEL targeted DCs produced only IL-2 and no IFN.γ IL4 or IL-10 and thus failed to polarize to Th1 or Th2 phenotype. (FIG. 4B and not shown). Therefore 3A9 cells proliferate in response to αDEC/HEL targeted DCs in vivo, but the T cells do not produce effector cytokines or polarize to Th1.

Although there was persistent expansion of 3A9 T cells in regional LNs and spleen 7 and 20 days after challenge with HEL peptide in CFA (FIG. 4C, spleen not shown), few 3A9 T cells survived in the LNs or spleen after exposure to antigen delivered by a DEC/HEL. The loss of 3A9 T cells was Fas independent as it also occurred with 3A9/lpr T cells (FIG. 4C). Thus, the initial expansion of T cells in response to antigen presented by DCs in vivo is not sustained, and most of the initial responding T cells disappear from lymphoid organs by day 7. These cells are either deleted or persist in extravascular sites (R. L. Reinhardt, A. Khoruts, R. Merica, T. Zell, M. K. Jenkins, Nature 410, 101-5 (2001). If they do persist outside lymphoid tissues they must be anergic, because they cannot be activated by further exposure to antigen, including peptide in CFA (FIG. 3D).

Detailed description of FIG. 4: CD4⁺ T cells divide in response to antigen presented by DCs in vivo, produce Il-2 but not IFN .gamma., and are then rapidly deleted. (A) CFSE labeled CD45.1⁺ 3A9 T cells were transferred into B10.BR and 24 hours later, the recipients were injected subcutaneously in the footpads with α DEC/HEL (0.2 μg), GL117/HEL (0.2 μg), HEL peptide in CFA or PBS. CD4⁺ T cells were purified by negative selection from regional lymph nodes. Three days after challenge with antigen they were analyzed by flow cytometry using antibodies specific for CD45.1 (A20), CD4 (L3T4) (both from PharMingen) and 3A9 T cell receptor (1G12). The plots show staining with 1G12 anti-3A9 and CFSE intensity on gated populations of CD4⁺CD45.1⁺ cells. The numbers indicate the percentage of CFSE high (undivided) and CFSE low (divided) CD4⁺ T cells. The results are from one of two similar experiments. (B) T cells produce 11-2 but not IFN-γ in response to antigens presented on DCs under physiological conditions. 3A9 cells were transferred into B10.BR mice and 24 hours later the recipients were injected subcutaneously in the footpads with α DEC/HEL (0.2.mu.g), GL117/HEL (0.2 μg), HEL peptide in CFA. CD4⁺. After 3 days T cells were purified by negative selection from regional lymph nodes as described in FIG. 3 and were stimulated for 4 hours with leukocyte activation cocktail (PharMingen). Cells were stained with antibodies specific for CD4 (L3T4) and 3A9 T cell receptor (1G12 ref). Fixed and permeabilized cells were then analyzed by flow cytometry using anti-IL-2-APC (JES6-5H4) and anti-IFN-γ PE (XMG1.2) (PharMingen). Histograms show staining with anti-IL-2 and anti-IFN-γ on gated populations of 3A9+CD4+cells. The thick lines indicate PBS control. (C) Same as in (A) but analysis performed 7 or 20 days after antigen administration.

DCs can be stimulated to increase their antigen presenting activity and their immunogenic potential by exposure to bacterial products or CD40L (C. Caux, et al., J Exp Med 180, 1263-72 (1994); K. Inaba, et al., J Exp Med 191, 927-36 (2000); F. Sallusto, A. Lanzavecchia, J Exp Med 1 19, 1109-18 (1994)), a TNF-family member expressed on activated CD4 T cells, platelets and mast cells (T. M. Foy, A. Aruffo, J. Bajorath, J. E. Buhlmann, R. J. Noelle, Annu Rev Immunol 14, 591-617 (1996)). To determine whether the combination of co-stimulators and antigen delivery to DCs produces persistent T cell activation, mice were injected with αDEC/HEL and the agonistic anti-CD40 antibody FGK 45 (A. Rolink, F. Melchers, J. Andersson, Immunity 5, 319-30 (1996)). In contrast to αDEC/HEL, the combination of αDEC/HEL and FGK 45 induced persistent T cell activation (FIG. 5B). The level of T cell activation seen with αDEC/HEL and FGK 45 at day 7 was comparable to αDEC/REL at day 2 or HEL peptide in CFA at day 2 and 7 (compare FIGS. 3B and 5B). To determine whether anti-CD40 treatment altered 3A9 T cell numbers in αDEC/HEL treated mice, we performed adoptive transfer experiments with CD45.1 allotype-marked T cells and assayed by flow cytometry. Whereas FGK 45 alone showed no effect on the number of 3A9 T cells in LNs at day 7, the combination of FGK 45 and αDEC/HEL induced persistent .about.8-10 fold expansion of 3A9 T cells, an increase similar to that seen with HEL peptide in CFA at day 7 (FIG. 5A and FIG. 4). We conclude that persistent T cell responses can be induced by antigen delivered to DCs in vivo if an additional activation signal such as CD40 ligation is provided.

To determine if CD40 ligation induced detectable phenotypic changes on DCs in our system, we analyzed DCs from mice transferred with 3A9 cells and injected with FGK 45 and α.DEC/HEL. Consistent with work by others we found that those DCs up-regulated their surface expression of CD40 and CD86 (FIG. 5C) (F. Koch, et al., Journal of Experimental Medicine 184, 741-6 (1996). This increase was more pronounced in the presence of antigen specific T cells suggesting a positive feedback mechanism between activated DCs and T cells (FIG. 5C).

Detailed description of FIG. 5: CD40 ligation prolongs T cell activation in response to antigens delivered to DCs and induces up-regulation of co-stimulatory molecules on DCs. (A) CD40 ligation induces persistent expansion of 3A9 cells in response to antigens delivered to DCs. CD45.1⁺3A9 T cells were transferred into B 10.BR mice and 24 hours later the recipients were injected subcutaneously in the footpads with 0.2 μg of a DEC/HEL alone or 90 μg of FGK45 or both or PBS. CD4⁺ T cells were purified by negative selection from regional lymph nodes 7 days after challenge with antigen and analyzed by flow cytometry using antibodies specific for CD45.1 and CD4 as described in FIG. 4. The numbers indicate the percentages of CD4⁺ CD45.1⁺ cells in LNs. (B) CD40 ligation prolongs T cell activation. 3A9 T cells were transferred into B 10.BR mice and 24 h later, recipients were injected subcutaneously in the footpads with 0.2 μg of. αDEC/HEL alone or 90 μg of FGK45 or both or PBS. After 2 or 7 days, CD4 T cells were isolated from the draining lymph nodes and cultured in vitro with irradiated B10.BR CD11c⁺ cells in presence or absence of HEL peptide. T cell proliferation was measured by ³H-thymidine incorporation. The results represent triplicate cultures from two independent experiments. (C) CD40 ligation induces co-stimulatory molecules on DCs. B 10.BR mice+/−3A9 cell transfer were injected with 90 μg FGK45+0.2 μg αDEC/HEL or α DEC/HEL or PBS. 3 days later DCs were isolated as in FIG. 2 and analyzed by flow cytometry using antibodies specific for CD11c, B220, CD86 (GL1-biot) and CD40 (HM40-3-FITC) (all from PharMingen). Histograms show staining with anti-CD40 and anti-CD86 on gated populations of DCs. Thick lines indicate control with PBS, which was same as a DECIHEL alone.

While the invention has been described and illustrated herein by references to the specific embodiments, various specific material, procedures and examples, it is understood that the invention is not restricted to the particular material combinations of material, and procedures selected for that purpose. Indeed, various modifications of the invention in addition to those described herein will become apparent to those skilled in the art from the foregoing description and the accompanying figures. Such modifications are intended to fall within the scope of the appended claim s.

Various publications are cited herein, the disclosures of which are incorporated by reference in their entireties.

### Materials and Methods for Examples 2-9

Antibodies and reagents. Alexa₄₈₈-conjugated αDEC-205 (NLDC-145), αOVA (3A11.1) and isotype control (III/10) antibodies were prepared using the Alexa Fluor® 488 Protein Labeling Kit (Molecular Probes).

Mice. Adult female C57BL/6 (B6) mice, and CD4^{-/-} and CD8^{-/-} B6 knockouts, were used were purchased, from Jackson Labs. OVA-specific, TCR transgenic CD45.1⁺ OT-I and CD45.1⁺ OT-II mice were used as described (20). DEC-205^{-/-} mice were generously provided by Dr. M. Nussenzweig (The Rockefeller University, New York, N.Y.).

Conjugation of OVA to monovalent monoclonal antibodies. Monovalent IgG's were conjugated to LPS-free OVA (Seikagaku Corporation, Japan) that had been activated with succinimidyl 4-{N-maleimidomethyl}cyclhexane-1-carboxylate (SMCC, Pierce) according to the manufacturer's protocol. Briefly, the antibodies were reduced using 100 mM 2-mercaptoethanesulfonic acid sodium salt (MESNA; Sigma) for 30 min at 37° C. and separated from the reducing agent over a desalting column. Then the activated OVA was mixed with the reduced antibodies overnight at 4° C. The antibody:OVA conjugates were passed over a protein G column to remove unconjugated OVA, concentrated by spin columns and evaluated by spectrophotometry and SDS-PAGE. Monovalent IgG:OVA conjugates were characterized by SDS-PAGE and Western blotting. Quantification of the OVA content of the conjugates was achieved by comparison with known quantities of OVA on the same blot detected with an HRP conjugated polyclonal rabbit anti-OVA antibody (Research Diagnostics, Inc.).

Purification of DCs and antigen specific T cells. Single cell suspensions were prepared from lymph nodes or spleen with 400 U/mL collagenase D (Roche) for 25 min and CD11c⁺ cells purified by MACS®. OVA-specific transgenic CD8⁺ or CD4⁺ T cells were prepared from lymph node or spleen cell suspensions of OT-I or OT-II mice using negative selection with hybridoma supernatants directed against MHC-II, F4/80, B220, NK 1.1, and CD4 or CD8 and goat anti-rat Dynabeads® (Dynal) at a ratio of 4 beads to 1 target cell.

Antigen targeting and maturation of DCs in vivo. Mice were injected s.c. in the paws with OVA protein, or Ig:conjugates of OVA protein, without or with a stimulus for DC maturation, which was the 1C10 agonisitic αCD40 antibody (Heath, A. W., W. W. Wu, and M. C. Howard. (1994), Eur. J. Immunol. 24:1828-1834) injected i.p. at 25-50 μg/mouse as described (Bonifaz, L., D. Bonnyay, K. Mahnke, M. Rivera, M. C. Nussenzweig, and R. M. Steinman. (2002), J. Exp. Med. 196:1627-1638).

Assays with TCR transgenic T cells to monitor antigen presentation on MHC class I and II products. In vitro antigen presentation assays were performed by adding CD11c⁺ DCs, selected from lymph nodes and spleens of OVA-treated mice, to 10⁵ OT-II or OT-II T cells in round bottom 96 well plates (1 DC:3 T cell ratio). At 48 hrs, ³H-thymidine (1 μCi, Amersham) was added for 12 hr to detect incorporation into DNA. In vivo assays were performed by injecting 10⁶ CD45.1⁺ OT-I or OT-II T cells that had been labeled at 10⁷ cells/mL with CFSE (Molecular Probes; 5 μM) for 10 min at 37° C.

Assays for OVA immunization. Proliferation of primed CD4⁺ or CD8⁺ T cells was evaluated by labeling bulk spleen suspensions with CFSE as above (but at 1 μM) and restimulating with LPS-free OVA (500 μg/mL) for 5 days in 24 well dishes at 2.5×10⁶ cells/well. Cultures were then washed, stained for CD4 and CD8 and evaluated for proliferation by flow cytometry. ELISPOT assays were performed by restimulating spleen suspensions for 2 days with H-2K^{b}-restricted peptide (SIINFEKL; 1.0 μM) or an I-A^{b}-restricted peptide (LSQAVHAAHAEINEAGR; 1.0 □M). The in vivo response of OVA specific CD8⁺ T cells was evaluated by staining with K^{b}-SIINFEKL:PE tetramers (kindly provided by Dr. E. Pamer, Memorial Sloan Kettering Institute) and CD62L for 1 hr at 4° C. Also IFN-γ producing effector cells were evaluated by culturing 5×10⁶ lymph node or spleen cells with SIINFEKL peptide (1.0 μM) for 6 hrs in the presence of brefeldin A (Sigma; 5 μg/mL). Cells were then harvested, stained for extracellular CD8 and then stained for cytokines with the BD Intracellular Cytokine Staining Starter Kit. In vivo CTL assays were performed as described (Ho, et al., (1994), J. Exp. Med. 179:1539-1549) by injecting 1:1 mixtures of peptide-pulsed and unpulsed syngeneic splenocytes (7×10⁶ each) and, 12-18 hrs later, specific lysis quantified as {(1−(ratio unprimed/ratio primed))×100}, with ratio determined as {% CFSE^{lo}/% CFSE^{hi}} (Wong, P., and E. G. Pamer. (2003), Immunity 18:499-511).

Vaccine induced resistance assays. Tumor challenges were performed with 5×10⁶ MO4, OVA-bearing B16 melanoma cells injected s.c. on the right flank either 7 days prior to, or 30-90 days after, immunization. Nontransduced B16 melanoma cells were used as controls to show that immunity was OVA-dependent. Challenge with recombinant vaccinia:OVA virus was performed with 10⁵ PFU applied intranasally as described (Brimnes, M. K., L. Bonifaz, R. M. Steinman, and T. M. Moran. (2003), J. Exp. Med. 198:133-144). 7 days later, lungs were harvested, extracts prepared by physical disruption, and viral titers evaluated by plaque forming assay on CV-1 cells. Tumor data are expressed as average tumor size from groups of at least 5 mice, while vaccinia titers as average±one S.D. for groups of at least 5 mice.

### Example 2

### Preparation of Monovalent αDEC-205:OVA Conjugates that More Efficiently Harness the Antigen Presenting Activity of DCs In Vivo

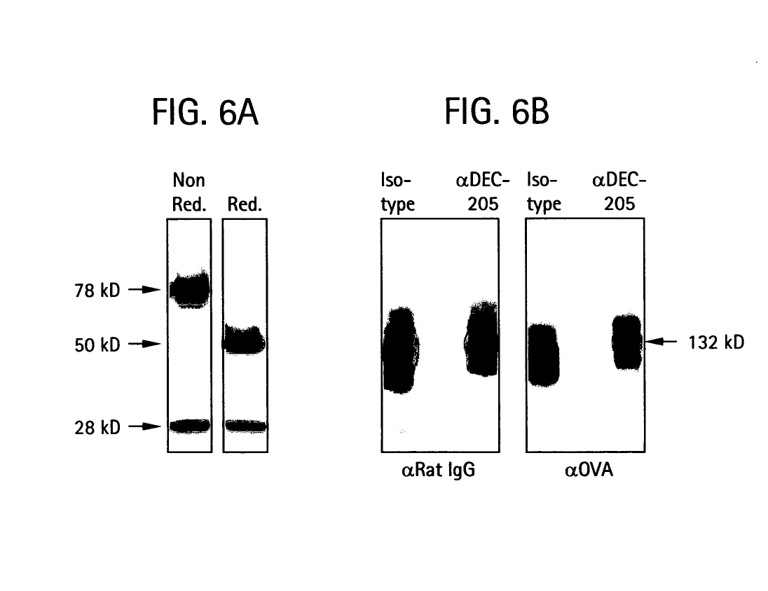

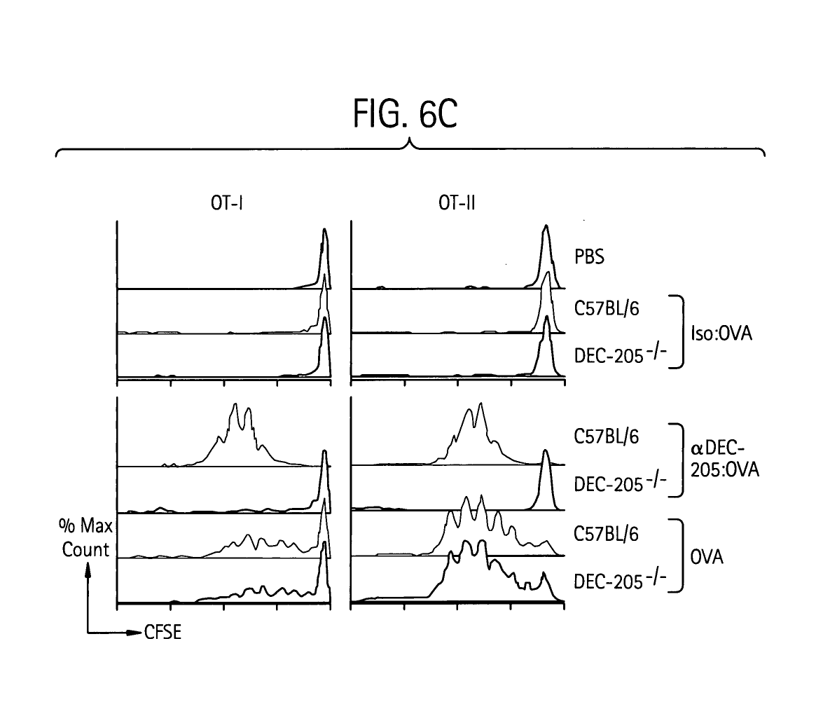

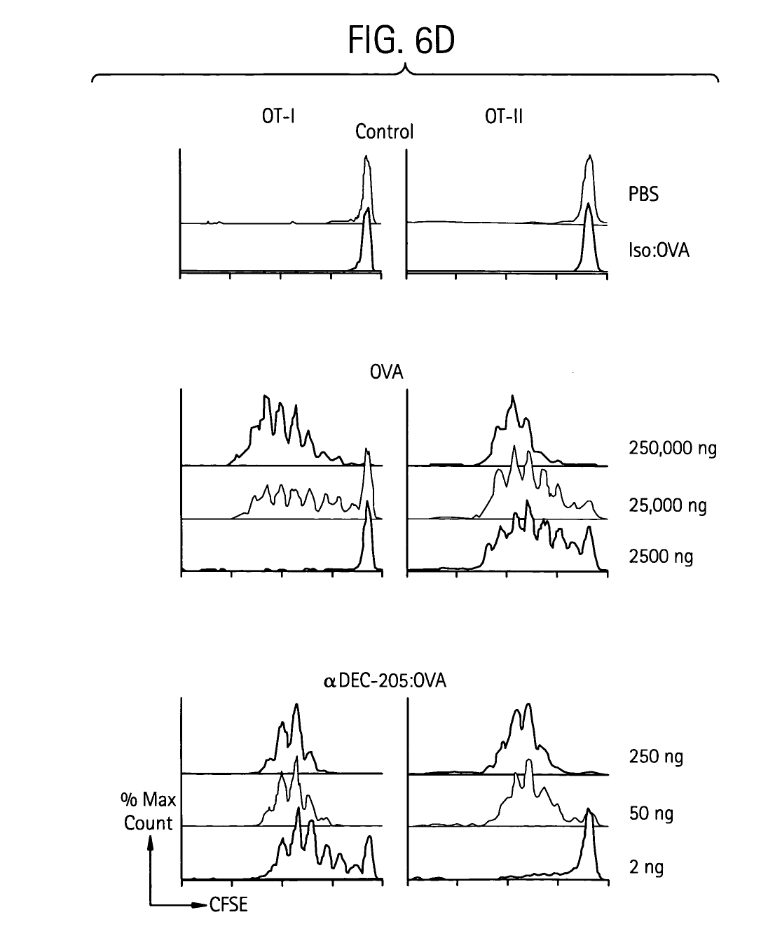

Modification of a prior strategy to conjugate an antigen to a monoclonal antibody to the DEC-205 receptor was utilized (Bonifaz, L., D. Bonnyay, K. Mahnke, M. Rivera, M. C. Nussenzweig, and R. M. Steinman. (2002), J. Exp. Med. 196:1627-1638.). The antibody used selectively targets to lymph node DCs following subcutaneous injection (Hawiger, D., K. Inaba, Y. Dorsett, K. Guo, K. Mahnke, M. Rivera, J. V. Ravetch, R. M. Steinman, and M. C. Nussenzweig. 2001. Dendritic cells induce peripheral T cell unresponsiveness under steady state conditions in vivo. J. Exp. Med. 194:769-780; Bonifaz, L., D. Bonnyay, K. Mahnke, M. Rivera, M. C. Nussenzweig, and R. M. Steinman. (2002), J. Exp. Med. 196:1627-1638.). With the mild reducing agent MESNA to cleave interheavy chain disulfide bonds, monovalent fragments of the antibody were produced (FIG. 6A). The exposed sulfhydryls of nearly all the antibody molecules could then be cross-linked with SMCC-activated ovalbumin (OVA, methods), yielding 132 kDa conjugates containing OVA and rat IgG (FIG. 6B). The αDEC-205:OVA conjugates, as well as conjugates produced with an isotype matched nonreactive antibody called III/10, were subjected to Western blotting along side known quantities of OVA protein to quantify the amount of OVA in the conjugates, generally about 10% of the total protein (data not shown). When the monovalent αDEC-205:OVA conjugates were injected subcutaneously, the OVA was presented to MHC-I and MHC-II restricted T cells in vivo, as assessed with OVA-specific reporter T cells from CD8⁺ OT-I and CD4⁺ OT-II, TCR transgenic mice. Both types of T cells, which were labeled with CFSE prior to injection, proliferated vigorously (5-7 division cycles) in response to αDEC-205:OVA but not to isotype matched III/10:OVA conjugates (FIG. 6C).

Results

When a comparison was made between the efficacy of achieving antigen presentation in vivo by monovalent antibody targeted OVA and soluble OVA, the conjugated OVA was >1000 times more effective for MHC class I presentation and >50 times greater for MHC class II presentation (FIG. 6C). For example, 2500 ng of soluble OVA did not elicit a proliferative response from CD8⁺ OT-I T cells, but 2 ng of αDEC-205:OVA caused most of the T cells to enter multiple cycles of division (FIG. 6C). In DEC-205 knockout mice (DEC-205^{-/-}), presentation of αDEC-205:OVA, but not soluble unconjugated OVA, was abolished (FIG. 6D), proving that presentation of αDEC-205:OVA was strictly dependent upon this endocytic receptor. Thus the injection of antigen conjugated to a monovalent αDEC-205 antibody markedly enhances the efficiency of antigen presentation in vivo.

### Example 3

### Immunization of CD4⁺ and CD8⁺ T Cells with a Combination of OVA Targeting to DCs and a CD40 Based Maturation Stimulus

Studies were done to determine if priming of the endogenous naïve repertoire, which contains a low frequency of antigen-specific T cells, could be accomplished. The induction of immunity to graded doses of antigen with two standard assays for immune priming: T cell proliferation in response to antigen and IFN-γ secreting Elispots was monitored. 500 ng of OVA conjugated to αDEC-205 (5 μg of antibody conjugate injected s.c. in 4 paws), in combination with 25 μg of αCD40 was injected.

Results

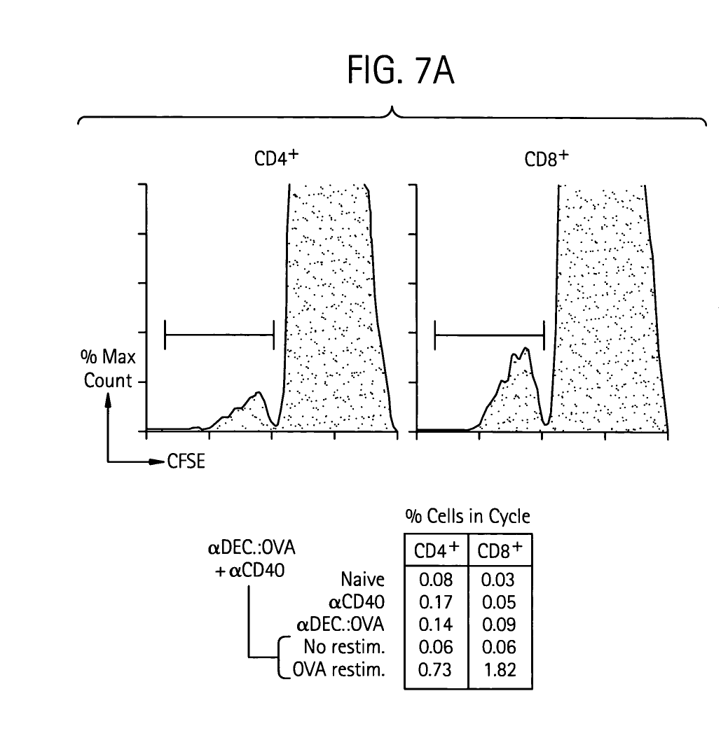

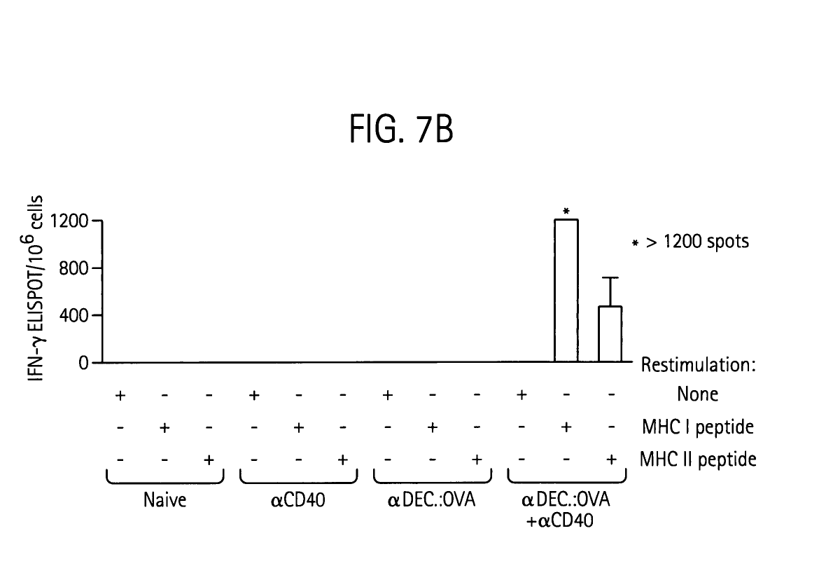

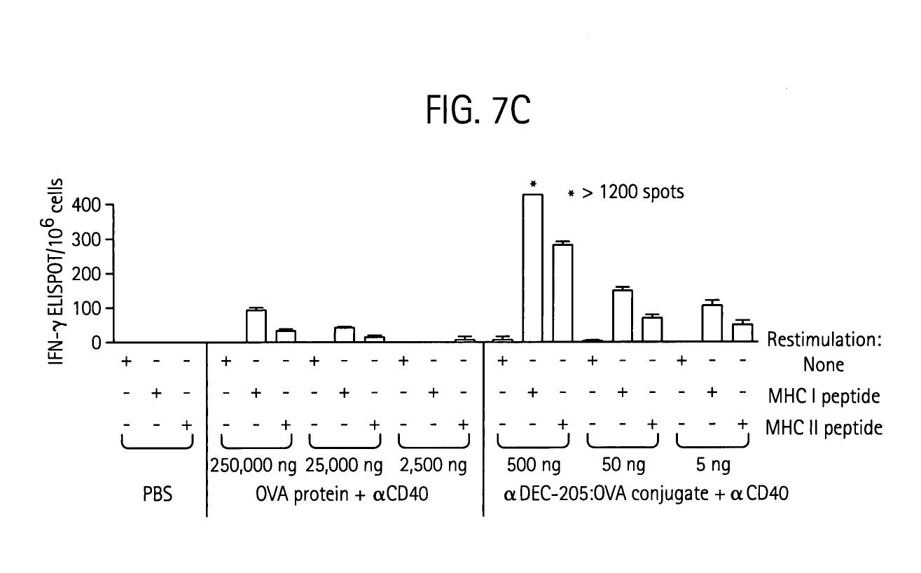

7 days after injection, both CD4⁺ and CD8⁺ T cells proliferated following in vitro restimulation with OVA protein (FIG. 7A). The proliferation was not detectable in control mice primed with αDEC-205:OVA or αCD40 alone (FIG. 7A). The mice also developed OVA-specific IFN-γ secreting effector cells, with the CD8⁺ response being more vigorous than the CD4⁺ response (FIG. 7B). When we used the T cell proliferation assay (data not shown) or elispot assay (FIG. 7C) to compare αDEC-205:OVA to OVA (each together with αCD40), the targeted antibody was >1000 times more effective for immunizing naive mice. Therefore antigen targeting to DCs via DEC-205, coupled with an αCD40 maturation stimulus, greatly increases the efficiency with which a protein initiates T cell mediated immunity from a polyclonal naive repertoire.

### Example 4

### The Durability of the Effector CD8⁺ T Cell Response when Antigen is Targeted to DCs

Subsequent studies were concentrated on the CD8⁺ response, because it is a special challenge to be able to present nonreplicating antigens to CD8⁺ T cells in vivo. Furthermore, this would be valuable for the design of safe non-replicating and subunit vaccines. A single dose of 50-100 ng of OVA conjugated to αDEC-205 (i.e., 0.5-1.0 μg of total antibody:OVA protein per mouse) together with 25 μg of agonistic αCD40 s.c., was administered and the development of effector T cells using assays for cytokine secretion and cytolytic activity was monitored.

Results

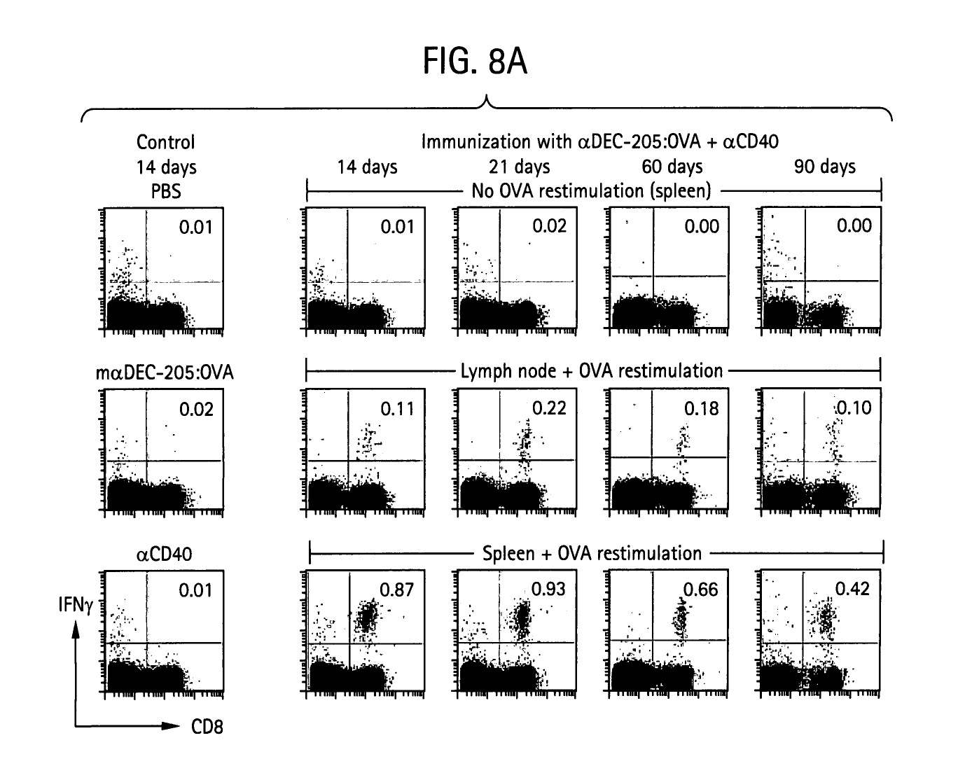

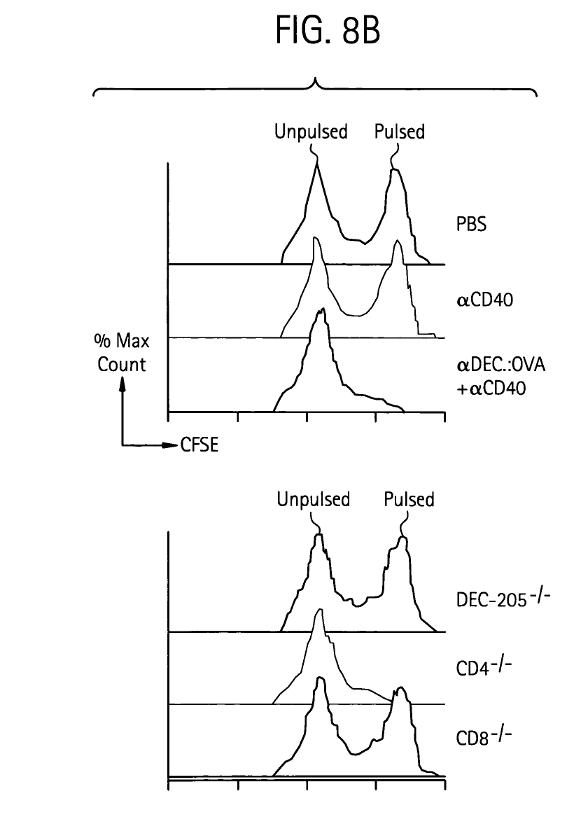

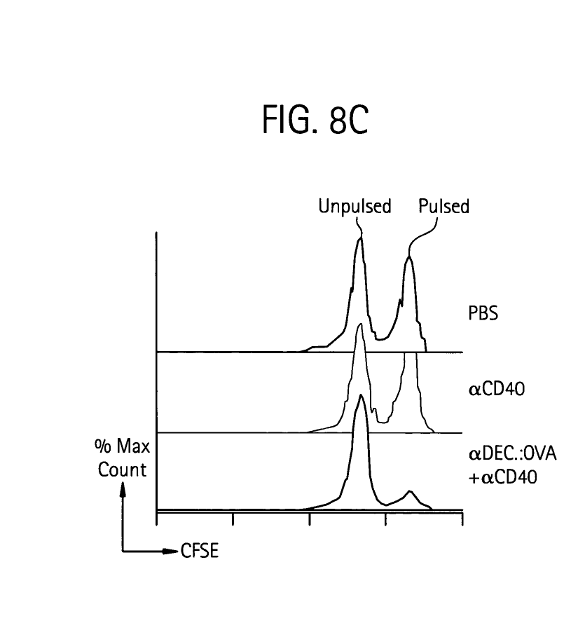

Antibody targeting to maturing DCs was able to elicit vigorous IFN-γ secretion by CD8⁺ T cells in both the lymph nodes and spleen, but in addition, the response was long lived (FIG. 8A). At all time points tested (14, 21, 60, 90 days) after administration of a single dose αDEC-205:OVA with αCD40, the CD8⁺ splenocytes had been primed to secrete IFN-γ upon peptide restimulation (FIG. 8A). Administration of either the antigen (αDEC-205:OVA) or DC maturation stimulus (αCD40) alone failed to elicit any response (FIG. 8A, left panels). To verify that the CD8⁺ response included cells with in vivo cytolytic function, we injected a mixture of peptide pulsed and unpulsed syngeneic splenocytes (7×10⁶ cells each) 14 days after immunization. Effective and specific CTLs were observed in the lymph nodes (FIG. 8B) and spleen (data not shown), with nearly all of the peptide-pulsed targets being eradicated from these organs. The CTL responses were undiminished in a CD4^{-/-} mouse, but completely absent in CD8^{-/-} mice and DEC-205^{-/-} mice (FIG. 8B). CTL activity remained vigorous 60 days following immunization (FIG. 8C, 77% lysis at day 60, compared to 93% lysis in FIG. 8B at day 14), and even 90 days post immunization, CTLs were still detected, although at lower levels (30% lysis; data not shown). These results indicate that a single immunization with αDEC-205:OVA and αCD40 leads to the durable formation of effector memory T cells.

### Example 5

### The Immune Response to αDEC-205:OVA is Greater than with Other Immunization Strategies

To compare the DC targeting strategy described herein with other immunization approaches that are commonly used to induce T cell mediated immunity to proteins, various techniques were studied:

i) splenic DCs matured and pulsed ex vivo with OVA (Mayordomo, J. I., T. Zorina, W. J. Storkus, L. Zitvogel, C. Celluzzi, L. D. Falo, C. J. Melief, S. T. Ilstad, W. M. Kast, A. B. DeLeo, and M. T. Lotze. (1995). Nat. Med. 1:1297-1302; Ludewig, B., S. Ehl, U. Karrer, B. Odermatt, H. Hengartner, and R. M. Zinkernagel. (1998), J. Virol. 272:3812-3818), as well as

ii) free antigens (OVA protein, OVA peptide and αDEC-205:OVA) suspended in Complete Freund's Adjuvant (CFA)(Le Bon, A., G. Schiavoni, G. D'Agostinio, I. Gresser, F. Belardelli, and D. F. Tough. 2001. Type I interferons potently enhance humoral immunity and can promote isotype switching by stimulating dendritic cells in vivo. Immunity 14:461-470) or given together with αCD40. 7 and 30 days after immunization, the expansion of OVA-specific T cells by MHC class I tetramer staining in lymph node and spleen was evaluated.

Results

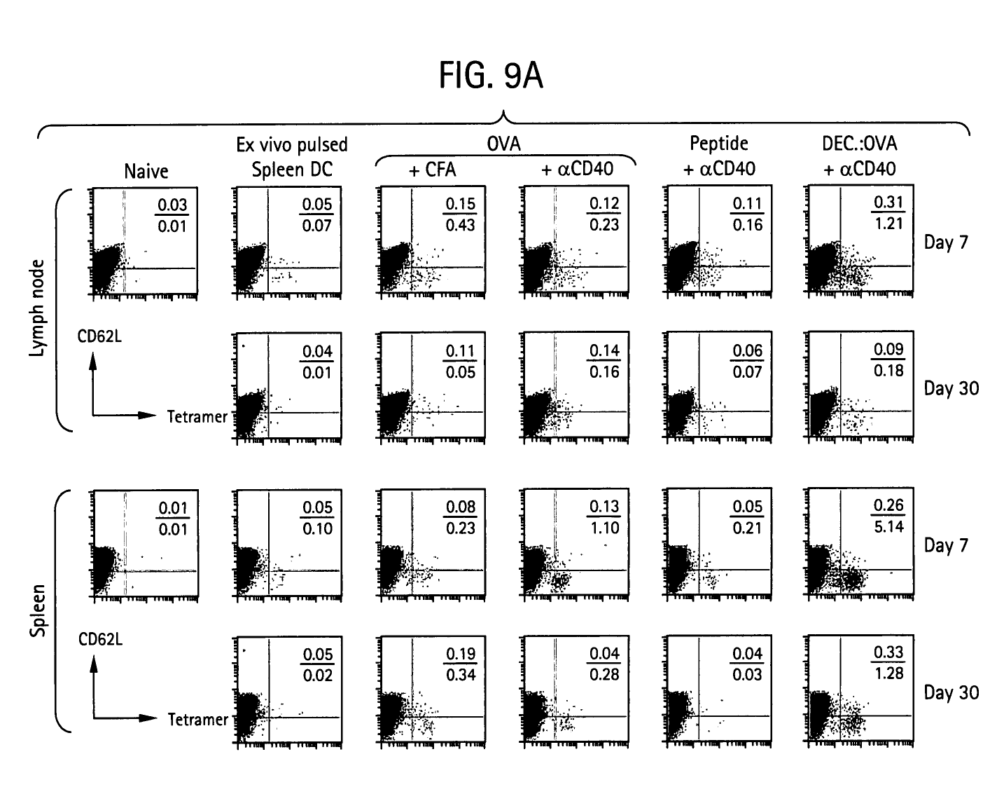

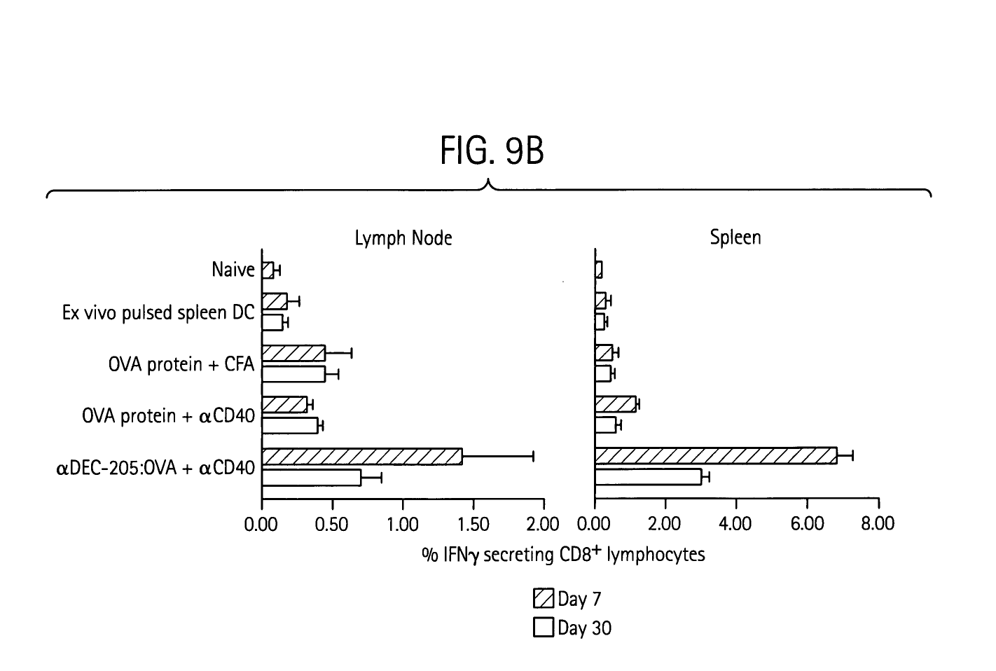

At both time points, the combination of αDEC-205:OVA with αCD40 was much more effective, especially if one examined the spleen, a site for the accumulation of effector memory T cells (FIG. 9A). The frequency of antigen-binding CD8⁺ cells was much higher in response to 50 ng of OVA conjugated to αDEC-205 (5.4%) relative to 50 μg soluble OVA, injected along with either αCD40 (1.2%) or CFA (0.3%); 50 μg preprocessed OVA peptide with αCD40 was even less effective (FIG. 9A). On day 7, the tetramer positive cells in the αDEC-205:OVA treated mice had downregulated CD62L confirming that these T cells were effectors (see FIG. 8) with the potential to migrate into peripheral tissues. The degree of expansion of tetramer positive cells correlated closely with the production of functioning effector cells assayed by IFN-γ secretion, which again was much higher following αDEC-205:OVA targeting relative to other forms of antigen delivery (FIG. 9B). These results indicate that direct in vivo delivery of protein antigens to DCs is more effective than several existing approaches for vaccine priming of antigen-specific CD8⁺ T cells.

### Example 6

### Systemic and Prolonged Distribution of OVA Following αDEC-205 Targeting to DCs

To determine how DEC-205 targeting improves antigen delivery in situ, the rate and persistence of antibody loading of DCs in lymphoid tissues were evaluated over time.

Results

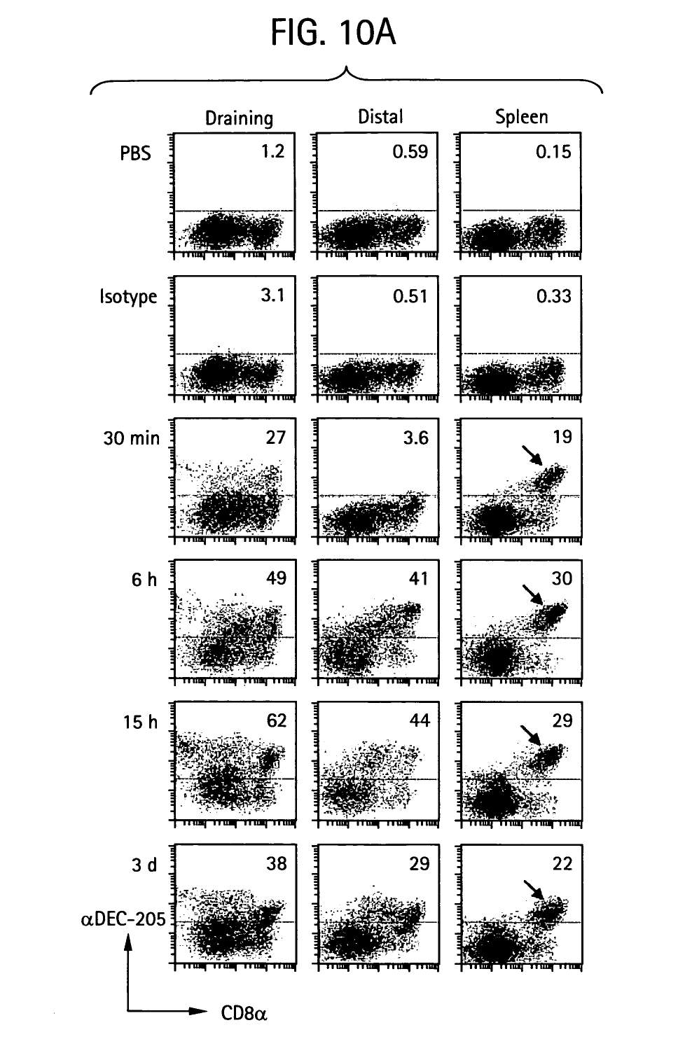

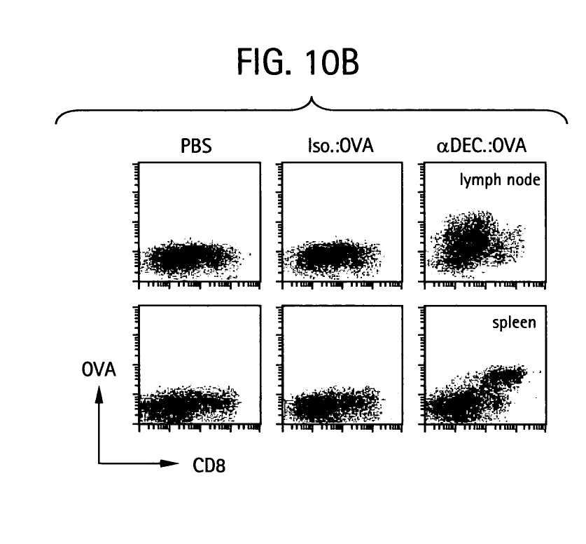

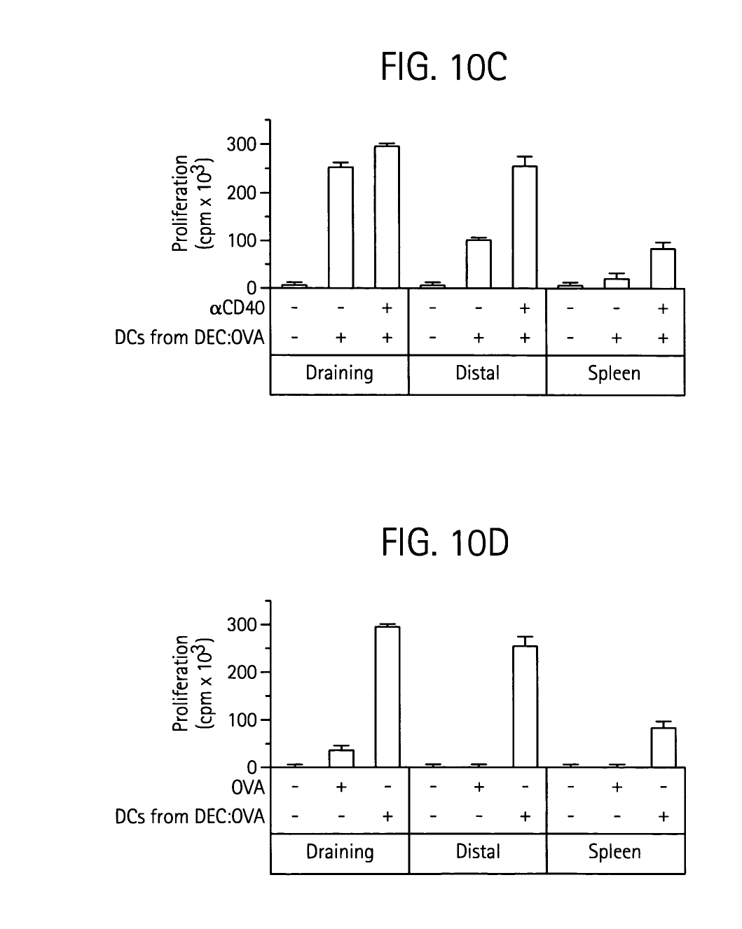

The isotype matched control III/10 antibody bound weakly if at all to DCs at all time points (FIG. 10A). In contrast, within 30 min of s.c. injection, Alexa₄₈₈® conjugated αDEC-205 began to load a sizable fraction of the CD11c⁺ DCs in the draining lymph nodes, consistent with the direct movement of antibody from the skin injection site via the protein-rich afferent lymph to the lymph node. Unexpectedly, the αDEC-205 quickly appeared on all of the CD8⁺ DCs of the spleen (the CD8⁺ DC subset is also the DEC-205 high subset in spleen although in lymph nodes, DEC-205 and CD8 expression are not coordinate on certain DC subsets (Vremec, D., and K. Shortman. (1997), J. Immunol. 159:565-573; Inaba, K., M. Pack, M. Inaba, H. Sakuta, F. Isdell, and R. M. Steinman. (1997), J. Exp. Med. 186:665-672), indicating that antibody was gaining access to the blood stream (FIG. 10A, arrows). Considerable loading in the mesenteric lymph node also was detectable, but at longer times after injection (FIG. 10A). By 6 hrs, αDEC-205 loaded at least 50% of the draining lymph node DCs and ˜40% and ˜30% in the distal lymph node and spleen DCs, respectively. Interestingly, αDEC-205 persisted on the DCs in all the organs for at least 3 days after injection (FIG. 10A, bottom). The presence of OVA in the DCs of a draining lymph node and spleen was also evident by intracellular staining for OVA (FIG. 10B). Isolation of the CD11c⁺ DCs from spleen and lymph nodes 15 hrs after injection of αDEC-205:OVA with or without αCD40 confirmed that these DCs could present the captured OVA to TCR transgenic T cells (FIG. 1C). When αDEC-205:OVA was compared to a 1000 fold higher dose of soluble OVA (each given together with αCD40), the former was presented much more vigorously by DCs from systemic lymphoid tissues (FIG. 10D). These results indicate that low doses of intracutaneous anti-DC antibodies rapidly target along with an associated antigen systemically to DCs in lymphoid tissues for days.

### Example 7

### Prolonged Presentation of MHC Class I-Peptide Complexes on Antigen-Targeted DCs

To investigate the persistence of MHC:OVA peptide complexes in vivo, mice were pre-treated with αDEC-205:OVA or OVA, each with or without aCD40, for 1, 3, 7, 15 or 30 days prior to transferring CFSE labeled OT-I OVA-specific T cells.

Results

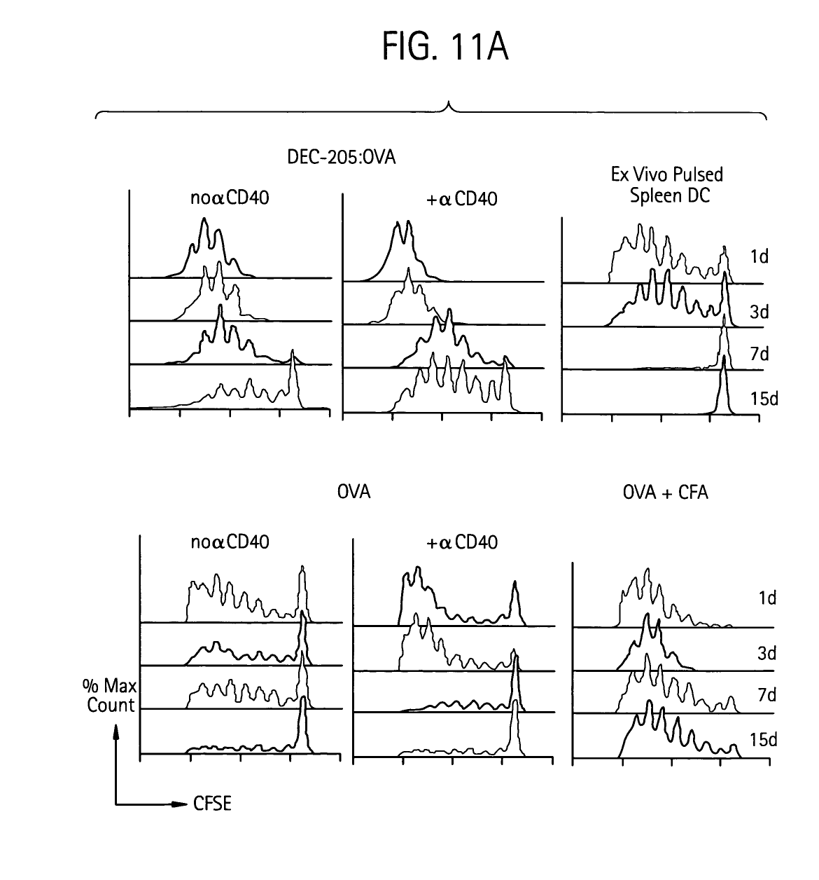

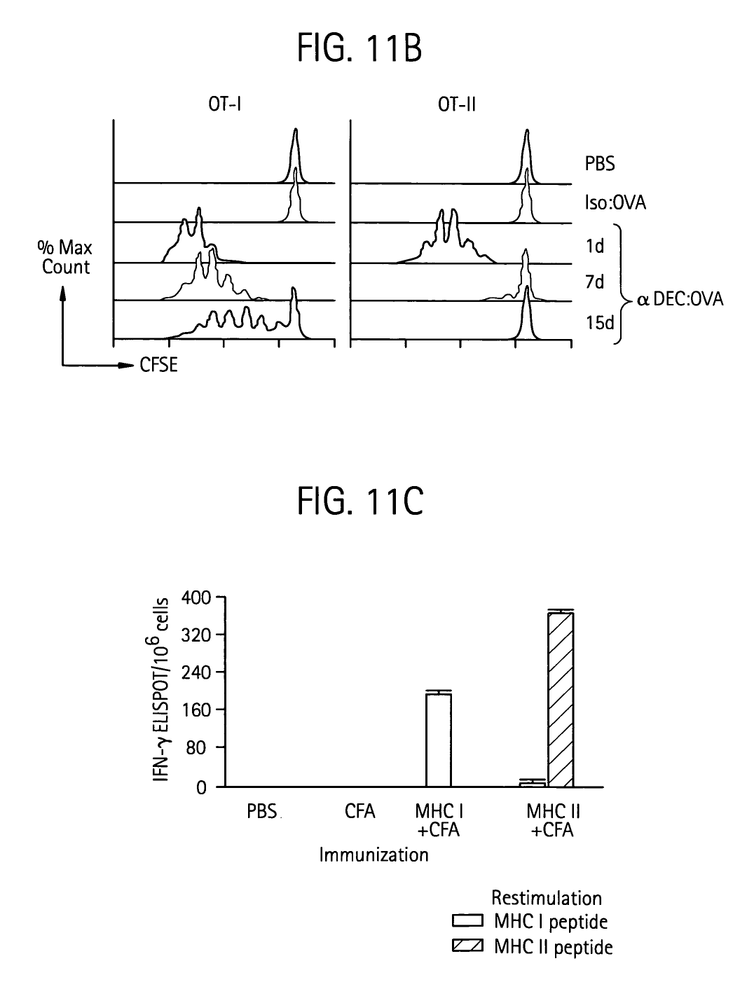

Surprisingly, given the evidence that the half life of DCs in lymph nodes is ˜1.5-2 days (Kamath, A. T., J. Pooley, M. A. O'Keeffe, D. Vremec, Y. Zhan, A. Lew, A. D'Amico, L. Wu, D. F. Tough, and K. S. Shortman. (2000), J. Immunol. 165:6762-6770; Kamath, A. T., S. Henri, F. Battye, D. F. Tough, and K. Shortman. (2002), Blood 100:1734-1741), presentation was still vigorous in the lymph nodes 15 days (but not 30 days; data not shown) after immunization with just 50 ng of OVA in αDEC-205:OVA conjugates (FIG. 11A, top left). Co-administration of αCD40 slightly increased the presentation, especially at day 15. In contrast, proliferation elicited by administration of 50 μg of soluble OVA or 50 μg of preprocessed peptide (not shown), was minimally detectable at 7 days after injection, even if co-administered with αCD40 (FIG. 11A, bottom left). Likewise, when mice were primed with ex vivo-loaded αCD40-matured splenic DCs, presentation was not detectable beyond 3 days after injection (FIG. 11A, top right). If a high dose of OVA protein (500 μg) was administered in CFA, proliferation also was detectable 15 days after administration (FIG. 11A, bottom right; as was the case for 50 ng of DEC-205 targeted OVA in CFA, data not shown), probably because the oily CFA emulsion allows the depot of injected antigen to persist. In contrast to MHC class I, MHC class II-peptide complexes were no longer detectable at 7 days after injection of αDEC-205:OVA (FIG. 11B). To test if the superiority of MHC class I presentation was due to an expanded OVA-specific CD8⁺ T cell repertoire, we immunized mice with preprocessed MHC class I and II binding OVA peptides. If anything the MHC class II restricted response was greater (FIG. 11C), suggesting that αDEC-205 targeting seems to prioritize presentation on MHC class I products. The results in FIGS. 5 and 6 indicate that the local injection of a single low dose of DC-targeted antigen recreates a situation analogous to a systemic infection, with prolonged presentation of antigen in most lymphoid tissues.

### Example 8

### DEC-205 Antigen Targeting as a Potential Vaccination Strategy for Resistance to Tumors

Resistance to a B16 melanoma stably transduced with OVA (termed MO4) was first studied. Protection studies were conducted in which vaccinated mice were challenged at a distal site with MO4 cells s.c., but this was done 2-3 months after a single vaccination to assess vaccine memory.

Results

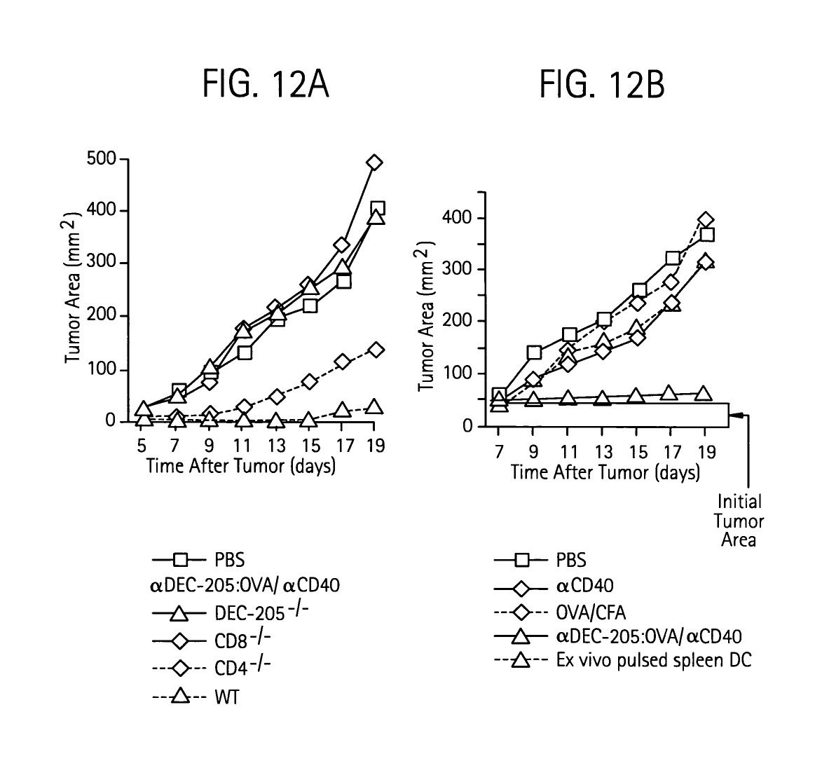

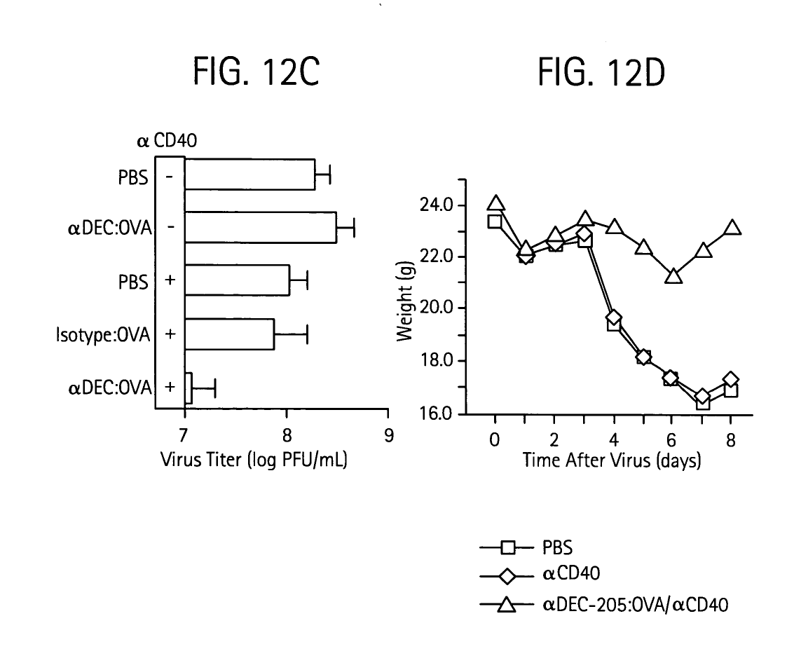

The mice that received αDEC-205:OVA conjugate in conjunction with αCD40 were protected against a subsequent administration of 5×10⁶ tumor cells 2-3 months later (FIG. 12A), while mice that received only one component of the vaccine (antigen or adjuvant) or the isotype conjugate were not (data not shown). This protection was specific for OVA, as the vaccinated mice were not protected against an identical tumor line (B16) that did not express OVA (data not shown). Studies with knockout mice determined that protection required DEC-205 expression, CD8⁺ T cells and, to a lesser extent, CD4⁺ T cells (FIG. 12A). DC targeting was then tested in a more demanding therapeutic assay, in which the OVA-bearing MO4 tumor cells were allowed to develop into 0.5-1.0 cm diameter tumors for 7 days prior to treatment with different strategies. The combination of αDEC-205:OVA in conjunction with αCD40 was able to induce a therapeutic effect, and this was much superior to other strategies, such as OVA in complete Freund's adjuvant and ex vivo loaded DCs (FIG. 12B).

### Example 9

### DEC-205 Targeting of Antigens as a Potential Vaccination Strategy for Mucosal Resistance

To evaluate if mucosal immunity could be established by this new systemic vaccination approach, mice were immunized with 50 ng of OVA conjugated to αDEC-205 together with αCD40 s.c., and 2 weeks later, the animals were challenged with intranasal recombinant vaccinia OVA.

Results

Protection was observed at a mucosal surface by measuring virus titres in the lung (FIG. 12C), but in addition, the mice did not lose weight as a result of infection (FIG. 12D). In contrast, no protection was observed relative to the PBS control if the animals had been vaccinated with either the isotype conjugate or αDEC-205:OVA or αCD40 alone (FIG. 12C). Therefore a single intracutaneous dose of only 50 ng of DC-targeted antigen is effective in generating protective immunity, including at a mucosal surface.

What is claimed is:

1. A method of promoting highly efficient antigen presentation in a mammal comprising:
a) exposing ex vivo or in vivo dendritic cells from said mammal to either of the following:
i) a conjugate comprising a preselected antigen covalently bound to an antibody to DEC-205; or
ii) a recombinant anti-DEC-205 antibody, wherein said antibody has been genetically modified to contain at least one preselected antigen on at least one preselected site on said antibody molecule; and
b) promoting maturation of said dendritic cells ex vivo or in vivo by combining the antigen/anti-DEC-205 complex of either of i) or ii) of step a) with a dendritic cell maturation factor;
wherein the combination of steps a) and b) results in highly efficient antigen presentation in said mammal.

2. A method of promoting highly efficient antigen presentation in a mammal comprising administering a recombinant anti-DEC-205 antibody to said mammal, wherein said antibody has been genetically modified to contain at least one preselected antigen and at least one dendritic cell maturation factor, each on at least one preselected site on said antibody, and wherein said administering results in delivery of said antigen to said dendritic cell, maturation of said dendritic cell and promotion of highly efficient antigen presentation.

3. The method of either of claims 1 or 2, wherein said preselected site on said antibody is on the heavy or light chain of said antibody, or on fragments thereof.

4. The method of either of claims 1 or 2, wherein said method results in induction of a long term cellular and/or humoral immune response in said mammal.

5. The method of claim 4, wherein said method results in said antigen being about 500 times more effective in inducing a long-lasting T cell response and in expanding antigen-specific CD4+ and CD8+ T cells in the mammal, as compared to an antigen administered without an anti-DEC-205 antibody and without a dendritic cell maturation factor.

6. The method of claim 4, wherein said method increases the efficiency with which the antigen initiates CD4+ and CD8+ immunity from the polyclonal naive T cell repertoire in vivo.

7. The method of either of claims 1 or 2, wherein said anti-DEC-205 antibody is a polyclonal or a monoclonal antibody.

8. The method of claim 7, wherein said antibody is selected from the group consisting of a human antibody, a murine antibody that reacts with human DEC-205 protein, a humanized antibody, and a human-chimerized antibody.

9. The method of claim 8, wherein said antibody is a monovalent or single chain antibody.

10. The method of claim 5, wherein the T cell response is selected from the group consisting of a cytolytic T cell response, a helper T cell response and a memory T cell response.

11. The method of either of claims 1 or 2, wherein said method results in priming of CD8+ T cells specific for the preselected antigen, and wherein said preselected antigen is a non-replicating and/or subunit vaccine.

12. The method of claim 11, wherein said vaccine is composed of antigens selected from the group consisting of a tumor vaccine, a viral vaccine, a bacterial vaccine and vaccines for other pathogenic organisms for which a long lasting immune response is necessary to provide long term protection from infection or disease.

13. The method of claim 12, wherein said viral vaccine is selected from the group consisting of a DNA viral vaccine, an RNA viral vaccine or a retroviral vaccine formed with the antibody combining function of the anti-DEC-205 antibody.

14. The method of claim 11, wherein said vaccine is administered as a single dose.

15. The method of claim 14, wherein said single dose is sufficient to elicit a long lasting immune response.

16. The method of claim 14, wherein said vaccine is effective when administered without adjuvant.

17. The method of claim 14, wherein said single dose of vaccine, when administered at levels of about 10 to 1000 fold lower than the level of a vaccine administered without an anti-DEC 205 antibody and without a dendritic cell maturation factor but with an adjuvant, results in highly efficient antigen presentation and induction of long lasting immune responses.

18. The method of claim 14, wherein said vaccine is administered at a single dose of about 1 mg to about 10 mg.

19. The method of claim 14, wherein said vaccine is administered at a single dose of about 1 μg to about 10 μg.

20. The method of claim 14, wherein said vaccine is administered at a single dose of about 10 ng to about 100 ng.

21. The method of any one of claims 11-20, wherein said vaccine is administered subcutaneously, intramuscularly, intravenously, intranasally, orally, mucosally, bucally or sublingually.

22. The method of claim 17, wherein said immune response is a cellular or humoral immune response.

23. The method of claim 22, wherein said cellular immune response is selected from the group consisting of a cytolytic T cell response, a helper T cell response and a memory T cell response.

24. A method for increasing the persistence of MHC class I: antigen complexes in a mammal comprising:
a) exposing ex vivo or in vivo dendritic cells from said mammal to either of the following:
i) a conjugate comprising a preselected antigen covalently bound to an antibody to DEC-205; or
ii) a recombinant anti-DEC-205 antibody, wherein said antibody has been genetically modified to contain at least one preselected antigen on at least one preselected site on said antibody molecule; and
b) promoting maturation of said dendritic cells ex vivo or in vivo by combining the antigen/anti-DEC-205 complex of either of i) or ii) of step a) with a dendritic cell maturation factor;
wherein the combination of steps a) and b) results in persistent presentation of antigen in the context of MHC class I antigens such that persistence of MHC class I: antigen complexes in said mammal results in induction of a long lasting T cell response specific for said antigen; and
wherein such persistent presentation of antigen is analogous to a systemic infection as evidenced by presentation of antigen in most lymphoid tissue.

25. The method of claim 24 wherein said MHC class I: antigen complexes persist in vivo in multiple lymphoid sites from about 15 to about 30 days.

26. The method of either of claims 1 or 2, wherein said method results in induction of mucosal immunity specific for said predetermined antigen.

27. The method of claim 12, wherein treatment of a mammal with said tumor vaccine results in tumor regression in vivo.

28. The method of claim 27, wherein said tumor regression is associated with an increase in a tumor specific CD8+ cytolytic T cell response.

29. A vaccine composition for inducing long term cellular or humoral immunity in a mammal comprising a mixture of:
a) an immunogenically effective amount of an antigen for which induction of long term cellular or humoral immunity is desired, said antigen prepared by either
i) conjugating said antigen with an anti-DEC-205 antibody; or
ii) utilizing a recombinant anti-DEC-205 antibody, wherein said antibody has been genetically modified to contain at least one preselected antigen on at least one preselected site on said antibody molecule;
b) a dendritic cell maturation factor;
c) a pharmaceutically acceptable adjuvant; and
wherein said vaccine composition is effective when administered at levels of about 10 to 1000 fold lower than the effective dose of a vaccine which is not conjugated to an anti-DEC-205 antibody or fragments thereof and which is not administered with a dendritic cell maturation factor, but for which an adjuvant is required.

30. An immunogenic composition, said composition comprising:
a) an immunogenically effective amount of an antigen for which induction of long term cellular or humoral immunity is desired, said antigen prepared by either
i) conjugating said antigen with an anti-DEC-205 antibody; or
ii) utilizing a recombinant anti-DEC-205 antibody, wherein said antibody has been genetically modified to contain at least one preselected antigen on at least one preselected site on said antibody molecule;
b) a dendritic cell maturation factor;
c) a pharmaceutically acceptable adjuvant;
d) a means for delivering said composition; and
wherein said composition results in generation of antigen specific antibodies and/or CD8+ cytolytic T cells, when administered at levels of about 10 to 1000 fold lower than the effective dose of a composition wherein the antigen is not conjugated to an anti-DEC-205 antibody or fragments thereof and which is not administered with a dendritic cell maturation factor, but which requires administration with an adjuvant.

31. A DNA vaccine composition comprising:
a) an isolated DNA molecule comprising at least one nucleotide sequence encoding at least one antigenic polypeptide isolated from a virus, bacterium or tumor cell against which immunity is desired;
b) an isolated DNA molecule comprising at least one nucleotide sequence encoding an anti-DEC-205 antibody or a DEC-205 binding fragment thereof;
c) a pharmaceutically acceptable carrier; and
wherein said composition, when administered with a dendritic cell maturation factor at levels of about 10 to 1000 fold lower than the effective dose of an antigenic polypeptide which is not conjugated to an anti-DEC-205 antibody or fragments thereof and which is not administered with a dendritic cell maturation factor, but requires an adjuvant, results in efficient, vigorous and long lasting cellular and humoral immunity specific for said virus, bacterium or tumor cell.

32. The composition of claim 31, wherein said nucleotide sequence encoding an anti-DEC-205 antibody or fragment thereof is selected from the nucleotide sequences set forth in SEQ ID NOS: 13 and 14, wherein said nucleotide sequences encode the heavy or light chain variable region of an anti-DEC-205 antibody.

33. A method for immunizing a mammal, comprising administering to said mammal a composition of any one of claims 29, 30 or 31.

34. A method for protection of a mammal from infection with a pathogen or a tumor cell comprising administering an immunogenically effective amount of a vaccine comprising:
a) a vector containing a gene encoding a protein or polypeptide from a pathogen or tumor cell or an immunogenic fragment thereof, operatively associated with a promoter capable of directing expression of the gene in the mammal; and
b) a vector containing a gene encoding the light or heavy chain anti-DEC-205 antibody operatively associated with a promoter capable of directing expression of the gene in the mammal;
c) a vector containing a gene encoding a dendritic cell maturation factor, operatively associated with a promoter capable of directing expression of the gene in the mammal; and
d) a pharmaceutically acceptable adjuvant.

35. A method for long term protection of a mammal from infection with a pathogen or a tumor cell comprising administering an immunogenically effective amount of a vaccine comprising:
a) a vector containing a gene encoding a protein or polypeptide from a pathogen or tumor cell or an immunogenic fragment thereof, operatively associated with a promoter capable of directing expression of the gene in the mammal;
b) a vector containing a gene encoding the light or heavy chain of an anti-DEC-205 antibody operatively associated with a promoter capable of directing expression of the gene in the mammal;
c) a pharmaceutically acceptable adjuvant; and
wherein said method further comprises administering the components of steps a), b) and c) with a dendritic cell maturation factor, wherein said administering results in long term protection of a mammal from infection with a pathogen or tumor cell.

36. A virus-like particle (VLP) comprising:
a) at least one immunogenic polypeptide from a virus against which immunity is desired conjugated to monovalent fragments of an anti-DEC-205 antibody;
b) a dendritic cell maturation factor;
c) a pharmaceutically acceptable adjuvant; and
wherein said virus like particle, when administered at an immunogenically effective amount with a dendritic cell maturation factor at levels of about 10 to 1000 fold lower than the effective dose of a virus-like particle which contains at least one immunogenic polypeptide from a virus against which immunity is desired and which is not conjugated to an anti-DEC-205 antibody or fragments thereof and which is not administered with a dendritic cell maturation factor, results in efficient and long lasting cellular and humoral immunity specific for said virus.

37. The virus-like particle of claim 36, wherein the at least one immunogenic polypeptide is obtained from a virus selected from the group consisting of a DNA virus, an RNA virus and a retrovirus.

38. A method of immunizing an animal against a virus, comprising administering an immunogenically effective amount of a virus-like particle of claim 36, wherein said immunizing results in induction of long term T cell, B cell or mucosal immunity.

39. A method for long term protection of a mammal from infection with a virus, said method comprising administering an immunogenically effective amount of a virus-like particle of claim 36.

40. A recombinant immunogenic composition comprising a nucleic acid molecule comprising:
a) a first nucleotide sequence encoding a chain of an antibody specific for DEC-205;
b) a second nucleotide sequence encoding at least one antigen from a virus, a bacterium, or a tumor cell against which immunity is desired;
c) a third nucleotide sequence encoding a dendritic cell maturation factor;
d) a fourth nucleotide sequence comprising a promoter for expression of a fusion protein comprising said anti-DEC-205 antibody, said antigen and said dendritic cell maturation factor; and
e) a pharmaceutically acceptable carrier.

41. The composition of claim 40, wherein said anti-DEC antibody is a polyclonal antibody, a monoclonal antibody, a chimeric antibody or monovalent fragments thereof.

42. The composition of claim 40, wherein said antibody chain is the light chain or heavy chain or fragments thereof.

43. The composition of claim 40, wherein said antibody is selected from the group consisting of a human or humanized antibody, a mouse antibody, a rat antibody, a horse antibody, a goat antibody, a sheep antibody, and monovalent fragments thereof.

44. The recombinant composition of claim 40, wherein transcription of the first, second and third nucleotide sequences are under the control of one promoter.

45. The composition of claim 40, wherein transcription of the first, second and third nucleotide sequences are under the control of individual promoters.

46. The method of any one of claims 1, 2, 11, 24, 26, 27, 29, 30, 31, 33, 34, 35, 36, or 40, wherein said dendritic cell maturation factor is selected from the group consisting of an anti-CD40 antibody, an inflammatory cytokine, poly I/C, single strand RNA, DNA, CpG, ligation of the 1L-1, TNF or TOLL-like receptor families, and activation of an intracellular pathway leading to dendritic cell maturation such as TRAF-6 or NF-κB.

47. The composition of any one of claims 29, 30, 31 or 40, wherein said composition is administered subcutaneously, intramuscularly, intravenously, intranasally, orally, mucosally, buccally, or sublingually.

48. The vaccine composition of any one of claims 29, 30, 31 or 40, wherein said composition induces long term T cell, B cell or mucosal immunity in a mammal.

49. The compositions of any one of claims 29, 30, or 31, wherein said antigen is selected from the group consisting of a viral antigen, a bacterial antigen, a tumor antigen and any other antigen obtained from a pathogenic organism or parasite for which long term T cell, B cell or mucosal immunity is desired.

50. A method for protection of a mammal from infection with a virus, a parasite, a bacterium, or a tumor, comprising administering an immunogenically effective amount of a composition of any one of claims 29, 30, 31 or 40.

51. A method of immunizing a mammal, comprising administering to said mammal an immunogenically effective amount of a composition of any one of claims 29, 30, 31 or 40.

52. A recombinant anti-DEC-205 molecule, comprising an antibody reactive with DEC-205 which has been genetically modified to contain at least one preselected antigen on at least one site on said antibody molecule, and at least one dendritic cell maturation factor on at least one site on said antibody molecule, wherein said antibody molecule, upon administration to a mammal, is capable of delivering said antigen to antigen presenting cells expressing DEC-205 and wherein said delivery results in highly efficient antigen presentation and induction of long term cellular and humoral immunity.

53. The antibody of claim 52, wherein the at least one site may be on either the heavy chain or the light chain.

54. The antibody of claim 53, wherein the heavy or light chain may be selected from the group consisting of the sequences set forth in SEQ ID NOS: 13 and 14.
